(19) United States
(12) Patent Application Publication
(10) Pub. No.: US 2023/0191061 A1
(43) Pub. Date: Jun. 22, 2023

(54) SYSTEMS, APPARATUS, AND METHODS FOR PERFORMING A PERCUTANEOUS TRACHEOSTOMY

(71) Applicant: CoapTech, Inc., Baltimore, MD (US)

(72) Inventors: Steven P. TROPELLO, Baltimore, MD (US); Aman GARG, Seattle, WA (US); William Alexander LeMay PATTERSON, Dundalk, MD (US)

(73) Assignee: CoapTech, Inc., Baltimore, MD (US)

(21) Appl. No.: 18/084,390

(22) Filed: Dec. 19, 2022

Related U.S. Application Data
(63) Continuation of application No. PCT/US2021/038263, filed on Jun. 21, 2021.
Application No. PCT/US2021/038263 is a continuation-in-part of application No. 17/108,200, filed on Dec. 1, 2020.
Application No. 17/108,200 is a continuation-in-part of application No. PCT/US2019/034943, filed on May 31, 2019.
(60) Provisional application No. 63/112,921, filed on Nov. 12, 2020.
(60) Provisional application No. 63/040,921, filed on Jun. 18, 2020.
(60) Provisional application No. 62/728,450, filed on Sep. 7, 2018.
(60) Provisional application No. 62/679,282, filed on Jun. 1, 2018.

(51) Int. Cl. A61M 16/04

(52) U.S. Cl. CPC A61M 16/0472; A61M 25/09; A61M 2205/3375

(57) ABSTRACT

Devices, systems, and methods for creating a percutaneous tracheostomy are described herein. In some embodiments, a system can include an inflation assembly and a guidewire assembly. The inflation assembly can include an elongated tube, an inflatable member, and a magnetic member. The elongated tube can have a first end, a second end, and can define a lumen. The inflatable member can be coupled to the first end of the elongated tube and can be fluidically coupled to the lumen such that the inflatable member can receive fluid via the lumen. The magnetic member can be coupled to the first end of the elongated tube. The first end of a guidewire of the guidewire assembly can include a coupling member, the coupling member configured to couple to the inflatable member such that translation of the elongated tube translates the guidewire assembly.

### CROSS-REFERENCE TO RELATED APPLICATIONS

This application is a continuation of PCT/US2021/038263, filed Jun. 21, 2021, which is a continuation-in-part of and claims priority to and the benefit of U.S. Pat. Application No. 17/108,200, filed Dec. 1, 2020, entitled “Systems, Apparatus, and Methods for Performing a Percutaneous Tracheostomy,” which is a continuation-in-part of and claims priority to and the benefit of International Patent Application No. PCT/US19/34943, filed May 31, 2019, entitled “Systems, Apparatus, and Methods for Performing a Percutaneous Tracheostomy,” which claims priority to and the benefit of U.S. Provisional Application No. 62/679,282, filed Jun. 1, 2018, entitled “Systems, Apparatus, and Methods for Performing a Percutaneous Tracheostomy” and U.S. Provisional Application No. 62/728,450, filed Sep. 7, 2018, entitled “Systems, Apparatus, and Methods for Performing a Percutaneous Tracheostomy,” the entire contents of each of which are hereby expressly incorporated by reference for all purposes.

U.S. Pat. Application No. 17/108,200 also claims priority to and the benefit of U.S. Provisional Application No. 63/040,921, filed Jun. 18, 2020, entitled “Systems, Apparatus, and Methods for Performing a Percutaneous Tracheostomy” and U.S. Provisional Application No. 63/112,921, filed Nov. 12, 2020, entitled “Systems, Apparatus, and Methods for Performing a Percutaneous Tracheostomy,” the entire contents of each of which are hereby expressly incorporated by reference for all purposes.

This application is related to International Patent Application No. PCT/US19/29351, filed Apr. 26, 2019, entitled “Systems, Apparatus, and Methods for Placing a Guidewire for a Gastrostomy Tube,” International Patent Application No. PCT/US17/026141, filed Apr. 5, 2017, entitled “Method and Apparatus for Coaptive Ultrasound Gastrostomy,” and International Patent Application No. PCT/US14/34950, filed Apr. 22, 2014, entitled “Coaptation Ultrasound Devices and Methods of Use,” the entire contents of each of which are hereby expressly incorporated by reference for all purposes.

### BACKGROUND

Embodiments described herein relate to systems, apparatus, and methods for creating a percutaneous tracheostomy to provide access to a patient’s windpipe via a route through the patient’s neck.

A tracheostomy procedure may be beneficial for patients who suffer from medical conditions that impair their ability to breathe. A percutaneous tracheostomy procedure is often preferable over an open surgical tracheostomy procedure because it is safer and less expensive. A percutaneous tracheostomy procedure generally requires puncturing the anterior trachea of a patient and inserting a guidewire using the Seldinger technique. The entry site used for the puncture and subsequent guidewire insertion is typically between the first and second or second and third tracheal rings. To achieve this, several percutaneous tracheostomy techniques have been developed, including the Ciaglia technique (serial or one-step dilation), the Griggs technique (wire forceps), and the translaryngeal tracheostomy (“TLT” or Fantoni) technique.

Identifying the ideal site for tracheal puncture can be challenging, so clinicians often use advanced tools such as ultrasound and bronchoscopy to identify key anatomical landmarks to inform the ideal tracheal puncture location. Ultrasound can be used to identify tracheal rings, proximal blood vessels, and the thyroid isthmus, but can put patients receiving a tracheal puncture at risk for posterior tracheal wall damage due to lack of ability to visualize the posterior trachea. Bronchoscopes can obstruct air passages and inhibit air flow to a patient who already has an impaired ability to breath. Bronchoscopy is also known to have issues with sterility, reliability, and immediate availability, and can greatly increase the cost of a percutaneous tracheostomy procedure.

Thus, there is a need for systems, apparatus, and methods for performing a percutaneous tracheostomy which reduce risks to the patient and allows for the percutaneous tracheostomy to be quickly and easily performed.

### SUMMARY

Systems, apparatus, and methods for performing a percutaneous tracheostomy are described herein. In some embodiments, a system includes an inflation assembly and a guidewire assembly. The inflation assembly can include an elongated tube, an inflatable member, and a magnetic member. The elongated tube can have a first end, a second end, and can define a lumen. The inflatable member can be coupled to the first end of the elongated tube and can be fluidically coupled to the lumen such that the inflatable member can receive fluid via the lumen. The magnetic member can be coupled to the first end of the elongated tube such that movement of the magnetic member can cause corresponding movement of the first end of the elongated tube. The guidewire assembly can include a guidewire having a first end and a second end. The first end of the guidewire assembly can include a coupling member, the coupling member configured to couple to the inflatable member such that translation of the elongated tube translates the guidewire assembly.

### BRIEF DESCRIPTION OF THE DRAWINGS

FIG. 1 is a schematic illustration of a guidewire placement system, according to an embodiment.

FIGS. 2A and 2B are schematic illustrations of a front view and a cross-sectional side view, respectively, of a portion of human anatomy.

FIG. 2C is a schematic illustration of a cross-sectional side view of a portion of human anatomy engaged with a portion of the guidewire placement system of FIG. 1, according to an embodiment.

FIGS. 3A-3M are schematic illustrations of a guidewire placement system in various stages of operation, according to an embodiment.

FIG. 4 is a schematic illustration of a guidewire placement system, according to an embodiment.

FIG. 5 is a schematic illustration of a guidewire placement system, according to an embodiment.

FIG. 6 is a schematic illustration of a guidewire placement system, according to an embodiment.

FIG. 7 is a flow chart of a method, according to an embodiment.

FIG. 8 is an illustration of an endotracheal tube inserted into the trachea of a patient, between the vocal folds.

FIG. 9 is a schematic illustration of an endotracheal tube disposed in the trachea of a patient.

FIG. 10 is a schematic illustration of an endotracheal tube disposed in the trachea of a patient, with a guidewire placement system that has been delivered through the larynx by a paired delivery technique.

FIG. 11 is a schematic illustration of a guidewire placement system, according to an embodiment.

FIGS. 12A to 12E are schematic illustrations of a guidewire placement system in various stages of operation, according to an embodiment.

FIG. 13 is a flow chart of a method, according to an embodiment.

FIGS. 14A and 14B are illustrations of an inflamed larynx, with and without an endotracheal tube inserted through the larynx.

FIG. 15 is a table showing dimensions of different sizes of endotracheal tubes, suitable for a range of patients.

FIG. 16 is a schematic illustration of a guidewire placement system that has been delivered through a larynx of a patient relative to an endotracheal tube by a paired delivery technique, according to an embodiment.

FIG. 17 is a schematic illustration of a guidewire placement system that has been delivered through a larynx of a patient relative to an endotracheal tube by a paired delivery technique, according to an embodiment.

FIG. 18 is a schematic illustration of a portion of a guidewire placement system, according to an embodiment.

FIG. 19 is a schematic illustration of a portion of a guidewire placement system, according to an embodiment.

FIGS. 20 and 21 are schematic illustrations of a side view of guidewire placement system disposed in an upper trachea of a patient in various stages of operation, according to an embodiment.

FIG. 22 is a cross-sectional view of the of guidewire placement system of FIGS. 20 and 21.

FIG. 23 is a perspective view of an inflation assembly, according to an embodiment.

FIG. 24 is a front view of the inflation assembly of FIG. 23.

FIG. 25 is a cross-sectional view of the inflation assembly of FIG. 23.

FIG. 26 is a perspective cross-sectional view of the inflation assembly of FIG. 23.

FIG. 27 is a perspective cross-sectional view of an inflation assembly, according to an embodiment.

FIG. 28 is a side perspective view of the inflation assembly of FIG. 27.

FIG. 29 is a front perspective view of the inflation assembly of FIG. 27.

FIG. 30 is a rear perspective cross-sectional view of the inflation assembly of FIG. 27.

FIG. 31 is a perspective cross-sectional view of an inflation assembly, according to an embodiment.

FIG. 32 is a cross-sectional view of the inflation assembly of FIG. 31.

FIG. 33 is a perspective cross-sectional view of the inflation assembly of FIG. 31 with an inner tubular member of the inflation assembly not shown.

FIG. 34 is an end view of the inflation assembly of FIG. 31.

FIG. 35 is a front perspective view of an inflation assembly system, according to an embodiment.

FIG. 36 is a perspective cross-sectional view of the inflation assembly of FIG. 35.

FIG. 37 is a side view of the inflation assembly of FIG. 35.

FIG. 38 is a perspective view of an inflation assembly, according to an embodiment.

FIG. 39 is a first side cross-sectional view of the inflation assembly of FIG. 38.

FIG. 40 is a second side cross-sectional view of the inflation assembly of FIG. 38.

FIG. 41 is a perspective cross-sectional view of the inflation assembly of FIG. 38.

FIG. 42 is a first top cross-sectional view of the inflation assembly of FIG. 38.

FIG. 43 is a second top cross-sectional view of the inflation assembly of FIG. 38.

FIGS. 44-47 are a perspective view, a top view, a distal end view, and a side view, respectively, of an inflation assembly, according to an embodiment.

FIGS. 48-51 are a perspective view, a top view, a distal end view, and a side view, respectively, of an inflation assembly, according to an embodiment.

FIGS. 52-55 are a perspective view, a top view, a distal end view, and a side view, respectively, of an inflation assembly, according to an embodiment.

FIGS. 56-59 are a perspective view, a top view, a distal end view, and a side view, respectively, of an inflation assembly, according to an embodiment.

FIGS. 60-63 are a perspective view, a top view, a distal end view, and a side view, respectively, of an inflation assembly, according to an embodiment.

### DETAILED DESCRIPTION

In some embodiments, a system includes an inflation assembly and a guidewire assembly. The inflation assembly can include an elongated tube, an inflatable member, and a magnetic member. The elongated tube can have a first end, a second end, and can define a lumen. The inflatable member can be coupled to the first end of the elongated tube and can be fluidically coupled to the lumen such that the inflatable member can receive fluid via the lumen. The magnetic member can be coupled to the first end of the elongated tube such that movement of the magnetic member can cause corresponding movement of the first end of the elongated tube. The guidewire assembly can include a guidewire having a first end and a second end. The first end of the guidewire assembly can include a coupling member, the coupling member configured to couple to the inflatable member such that translation of the elongated tube translates the guidewire assembly.

In some embodiments, a method can include translating a tubular member through an orifice of a patient, through a cricoid ring of the patient, and into an upper trachea of the patient. A first end of an elongated tube can be translated through a lumen of the tubular member such that an inflatable member and a magnetic member of the elongated tube extends from a first end of the tubular member and is disposed in the upper trachea of the patient. An external magnetic assembly can be disposed on an anterior neck of the patient such that the magnetic member of the elongated tube is urged toward the anterior neck of the patient and the inflatable member is disposed against an inner surface of the upper trachea. The inflatable member can then be inflated via a lumen of the elongated tube such that the inflatable member transitions from an uninflated configuration to an inflated configuration. A coupling member of a guidewire assembly can be translated through the anterior neck of the patient and into the upper trachea of the patient. The guidewire assembly can include a guidewire having a first end coupled to the coupling member and a second end disposed outside the patient, the guidewire extending through the anterior neck of the patient. The coupling member can be coupled to the inflatable member.

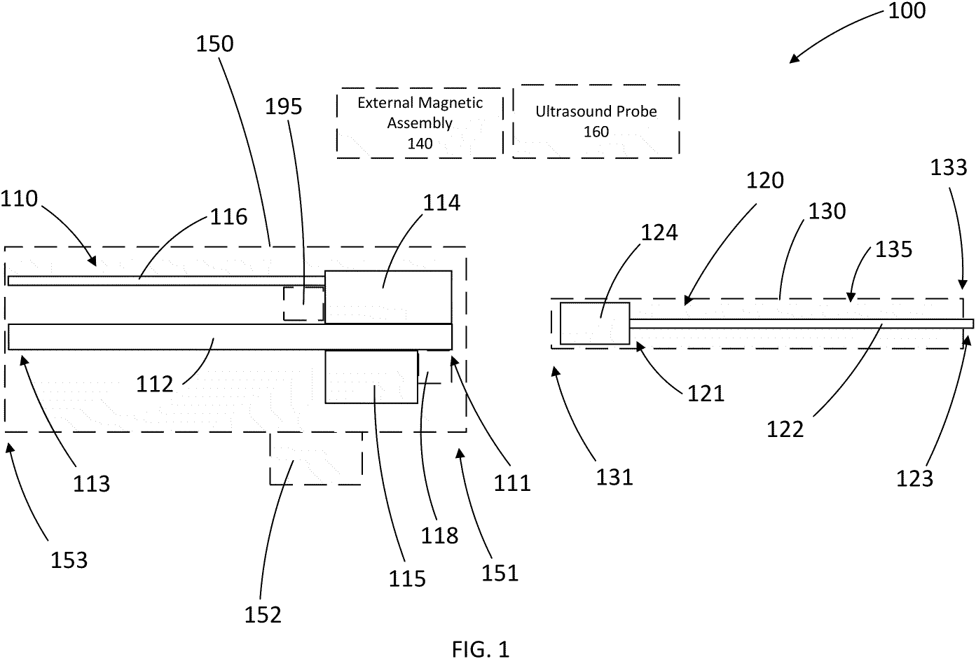

FIG. 1 is a schematic representation of a system 100. The system 100 includes an inflation assembly 110, a guidewire assembly 120, and a tubular member 150. The system 100 can optionally also include an external magnetic assembly 140 and an ultrasound probe 160. The inflation assembly 110 can include an elongated tube 112, an inflatable member 114, and a magnetic member 115. The inflation assembly 110 may optionally include a barrier member 195. The elongated tube 112 can have a first end 111 and a second end 113. In some embodiments, the elongated tube 112 can have a length sufficient to extend from at least an oral or nasal orifice of a patient to the trachea of the patient. The inflatable member 114 and the magnetic member 115 can be coupled to the elongated tube 112 at or near the first end 111 of the elongated tube 112. The inflation assembly 110 can include an inflation lumen 116 in fluid communication with the inflatable member 114. In some embodiments, the inflation lumen 116 can be disposed within and/or be defined by the elongated tube 112.

In some implementations, a light source 118 may be disposed on or near the first end 111 of the elongated tube 112. The light source 118 may produce sufficient light such that light can emit from the light source 118, through the tracheal wall, to the surface of the neck and be visible to a user (e.g., a clinician). Thus, the user may be able to determine the location of the first end 111 of the elongated tube 112 based, at least in part, on the location of light emitting through the patient’s skin. In some embodiments, the light source 118 and the tubular member 150 may be configured such that light emitted by the light source 118 is partially or fully blocked by the tubular member 150 when the first end 111 of the elongated tube 112 is disposed within the tubular member 150 such that the light emitted by the light member is not visible on the skin of the patient or is more dim compared to when the first end 111 of the elongated tube 112 is not within the tubular member 150. Thus, when the elongated tube 112 is translated relative to the tubular member 150 such that the first end 111 extends from the tubular member 150 when the elongated tube 112 and the tubular member 150 are at least partially inserted into the patient, the light emitted from the light source 118 may become visible or more visible when the first end 111 is extended from an end of the tubular member 150. The light source 118 can be, for example, a light emitting diode (LED).

The magnetic member 115 can be any suitable magnetic member configured such that movement of the magnetic member 115 causes corresponding movement of the first end 111 of the elongated tube 112. The magnetic member 115 can have any suitable shape. For example, in some embodiments, the magnetic member 115 can be shaped as an elongated rectangle. In some embodiments, the magnetic member 115 can be shaped as a cylinder. In some embodiments, the magnetic member 115 may be arcuate. In some embodiments, the magnetic member 115 is coupled directly to the elongated tube 112. In some embodiments, the magnetic member 115 is disposed within the inflatable member 114 and at least partially surrounded by the inflatable member 114. In some embodiments, the magnetic member 115 is coupled directly to the inflatable member 114. In some embodiments, the system 100 includes two or more magnetic members 115.

In some embodiments, the inflatable member 114 can surround the elongated tube 112 in an inflated and/or uninflated configuration. In some embodiments, the inflatable member 114 can extend laterally from the elongated tube 112 in an inflated and/or uninflated configuration. In some embodiments, the inflatable member 114 can extend distally from the first end 111 of the elongated tube 112 in an inflated and/or uninflated configuration. In some embodiments, the inflatable member 114 can be disposed on the elongated tube 112 such that a portion of the elongated tube 112 extends distally of the inflatable member 114 when the inflatable member 114 is in an inflated and/or uninflated configuration. In some embodiments, the inflatable member 114 can have two ends (e.g., cuffs), and each end can be sealed to an outer surface of the elongated tube 112. The elongated tube 112 can define one or more inflation ports or holes such that the inflation lumen 116 can be in fluid communication with the interior of the inflatable member 114 for transitioning the inflatable member 114 between an uninflated and an inflated configuration. In some embodiments, the inflatable member 114 can be formed on or as a part of a rigid subassembly, and the rigid subassembly can receive the elongated tube 112 within an orifice of the subassembly and the elongated tube 112 can then be sealed to the subassembly.

In some embodiments, the inflatable member 114 can be formed in any suitable shape, in any suitable size, and of any suitable material. For example, the inflatable member 114 can be elliptical, spherical, cylindrical, rectangular, tear drop, or any other suitable shape. In some embodiments, the shape can be chosen based on the particular application of the system 100. For example, the shape of the inflatable member 114 may be selected to improve ultrasound visualization in particular regions of a patient’s body. Furthermore, the inflatable member 114 can be sized for improved engagement and retention between the inflatable member 114 and the guidewire assembly 120.

The inflatable member 114 can be sufficiently pliable such that the inflatable member 114 (e.g., when inflated) can be punctured (e.g., by a needle) to define a pinhole in the wall of the inflatable member 114 rather than bursting or tearing as a result of puncture. In some embodiments, the inflatable member 114 can be formed of, for example, polyurethane, silicone, and/or polyvinyl chloride (PVC). In some embodiments, the inflatable member 114 can have any suitable material properties, wall thicknesses, and/ or inflated outermost diameters.

In some embodiments, for example, the inflatable member 114 can be elliptical in shape and formed of a low durometer urethane. The inflatable member 114 can have an outermost diameter ranging from about 5 mm to about 30 mm in an inflated configuration, and a length of about 20 mm. The wall thickness at the maximum balloon diameter in the inflated configuration can be between about 0.029 mm and about 0.038 mm. The inflatable member 114 can be filled with up to, for example, about 5 ml to about 10 ml of fluid in the inflated configuration.

The guidewire assembly 120 can include a guidewire 122 having a first end 121 and a second end 123 and a coupling member 124 disposed at the first end 121 of the guidewire 122. The coupling member 124 can be configured to couple to the inflatable member 114 such that, when coupled, translation of the inflation assembly 110 (e.g., translation of the elongated tube 112 via pulling on the second end 113) can translate the guidewire assembly 120. For example, if the inflatable member 114 is moved in a first direction due to a force applied to the elongated tube 112, the coupling of the coupling member 124 to the inflatable member 114 can cause the coupling member 124 and the guidewire 122 to also move in the first direction. The coupling member 124 can be configured to couple with the inflatable member 114 via, for example, being captured by the inflatable member 114, caught within an interior region of the inflatable member, or engaged with a surface of the inflatable member 114.

In some embodiments, the coupling member 124 can be distinct from the guidewire 122 and fixedly coupled to the guidewire 122 (e.g., via adhesive). For example, in some embodiments, the coupling member 124 can include a first magnetic member configured to couple to a second magnetic member of the inflatable member 114.

In some embodiments, the guidewire 122 can include the coupling member 124. For example, the coupling member 124 can be monolithically formed with a shaft of the guidewire 122 such that the guidewire assembly 120 is a one piece structure. Similarly, in some embodiments, the coupling member 124 and the guidewire 122 can be formed of the same material or materials. In some embodiments, the coupling member 124 can be shaped such that the coupling member 124 can engage with at least one portion of a wall of the inflatable member 114. For example, the coupling member 124 can have a planar or a multi-planar shape and can be formed as a pigtail, hook, coil, or corkscrew-shaped end to the guidewire 122. Thus, in some embodiments, the first end 121 of the guidewire 122 can be retained within or near the inflatable member 122 by the coupling member 124 when the coupling member 124 is disposed within the inflatable member 114. In some embodiments, the coupling member 124 can be disposed outside of the inflatable member 114 with the guidewire 122 passing through a first wall portion and a second, oppositely disposed wall portion of the inflatable member 114 such that the guidewire 122 is retained by the inflatable member 114 due to the interaction between the coupling member 124 and the first wall portion of the inflatable member 114. In some embodiments, the coupling member 124 can be partially disposed within the inflatable member 114 and partially disposed outside of the inflatable member 114 such that the guidewire assembly 120 is coupled to the inflatable member 114 for translation of the guidewire assembly 120 via movement of the inflation assembly 110.

In some embodiments, the coupling member 124 can be configured to transition between a first configuration for insertion and a second configuration for retention or coupling. For example, the coupling member 124 can have a smaller lateral extent (e.g., outermost diameter) relative to a central axis of the guidewire 122 in the first configuration than in the second configuration such that the coupling member 124 can fit inside the lumen 135 of the needle 130 in the first configuration and can expand to retain the guidewire 122 relative to the inflatable member 114 in the second configuration. In some embodiments, the coupling member 124 can have a first shape in the first configuration and a second shape in the second configuration such that the coupling member 124 can travel through an opening in at least one sidewall of the inflatable member 114 in the first configuration and can engage a sidewall of the inflatable member 114 in the second configuration such that the coupling member 124 is retained by the inflatable member 114. In some embodiments, the coupling member 124 can be biased toward the second configuration such that, in the absence of external forces on the coupling member 124, the coupling member 124 will assume the second configuration. In some embodiments, in the first configuration the coupling member 124 can be elongated such that the coupling member is shaped as a straight wire. The second configuration can correspond to an unbiased shape or configuration of the coupling member (e.g., a pigtail, hook, coil, or corkscrew-shape). In some embodiments, the guidewire 122 and/or the coupling member 124 can be formed of a shape-memory material such as, for example, Nitinol.

In some embodiments, when the coupling member 124 is within the lumen 135 of the needle 130, the needle 130 can compress the coupling member 124 such that the coupling member is in the first configuration. Thus, the coupling member 124 can have a smaller lateral extent relative to a central axis of the guidewire 122 (e.g., outermost diameter) when disposed within the lumen 135 of the needle 130 than when not within the needle 130. In some embodiments, the lumen 135 and the coupling member 124 can be structured and sized such that the coupling member 124 can be straight or substantially straight within the lumen 135 of the needle 130. For example, the lumen 135 can have an inner diameter similar to an outer diameter of the coupling member 124 (e.g., an outer diameter of a wire forming the coupling member 124 portion of the guidewire assembly 120) such that the coupling member 124 can be laterally compressed to a shape with a smaller outer diameter and/or elongated within the lumen 135 of the needle 130. In some embodiments, the outer diameter of a wire forming the coupling member 124 and the inner diameter of the lumen 135 can be relatively sized such that the outer diameter of the wire forming the coupling member 124 is slightly smaller than the inner diameter of the lumen 135 and the coupling member 124 and the inner surface of the needle 130 defining the lumen 130 can have a slip fit engagement. Thus, when the coupling member 124 is threaded into the lumen 135 of the needle 130, the wire forming the coupling member 124 is straightened out to correspond to the shape of the lumen 135. As the coupling member 124 is translated out of the first end 131 of the needle 130, the coupling member 124 can transition from the first configuration to the second configuration. For example, as the coupling member 124 is extended from the first end 131 of the needle 130, the portion of the coupling member 124 extending from the first end 131 can transition toward the second configuration due to being biased toward the second configuration, while the portion of the coupling member 124 remaining within the lumen 135 of the needle 130 can remain in the first configuration. When the coupling member 124 is entirely outside of the needle 130, the coupling member 124 can be entirely in the second configuration.

In some embodiments, the coupling member 124 can be configured to be translated in a first direction by the inflatable member 114 if a translation force on the inflatable member 114 (e.g., a translating force on the inflatable member 114 and/or a force holding the inflatable member 114 stationary) is greater than a force in a direction opposite of the translation force on the coupling member 124. If the force on the coupling member 124 is opposite and greater than the translation force on the inflatable member 114, the coupling member 124 and the inflatable member 114 can be configured to decouple. For example, in some embodiments in which the coupling member 124 is a pigtail-shaped end to the guidewire 122, the application of sufficient force to the coupling member 124 in a direction opposite a force being applied to the inflatable member 114 can cause the pigtail-shaped end to straighten and decouple from the inflatable member 114. In some embodiments, the application of sufficient force to the coupling member 124 in a direction opposite a force being applied to the inflatable member 114 may cause the coupling member to tear a sidewall of the inflatable member such that the inflatable member 114 and the coupling member 124 are decoupled. Thus, in some embodiments the coupling member 124 and the inflatable member 114 can be decoupled via applying oppositely directing pulling forces to each of the coupling member 124 and the inflatable member 114. In some embodiments, the coupling member 124 and the inflatable member 114 can be engaged such that the release force (e.g., via oppositely directing pulling forces) necessary to separate the coupling member 124 from the inflatable member 114 is a force greater than the maximum force applied to the guidewire 122 (and therefore coupling member 124) in an opposite direction than the inflatable member 114 during withdrawal of the coupling member 124 from the patient via pulling on the inflation assembly 110. Thus, the release force is sufficiently high such that the inflatable member 114 and the coupling member 124 will not be separated during the withdrawal of the coupling member 124 of the guidewire 122 inadvertently during withdrawal, but can be separated via, for example, pulling by the user when the inflatable member 114 and the coupling member 124 are outside of the patient’s body. For example, in some embodiments, the release force can be at least about 0.25 lbs of force, at least about 0.5 lbs of force, or at least about 1.5 lbs of force. In some applications of the system 100, the release force may be greater or smaller depending on the resistive forces the coupling member 124 and guidewire 122 may experience during withdrawal via a withdrawal force on the inflation assembly 110. In some embodiments, the coupling member 124 and the inflatable member 114 can be engaged such that the release force (e.g., via oppositely directing pulling forces) necessary to separate the coupling member 124 from the inflatable member 114 is a force less than the force applied to the guidewire 122 (and therefore coupling member 124) in a direction away from the patient (e.g., away from the patient’s anterior neck) during withdrawal of the inflation assembly 110 from the patient via a patient orifice via pulling on the elongated tube 112. Thus, the release force can be sufficiently low such that the inflatable member 114 and the coupling member 124 will not be inadvertently separated during the movement of the coupling member 124 of the guidewire 122 via movement of the inflatable member 114 within, for example, the trachea of the patient, but can be separated via, for example, pulling by the user on the elongated tube 112 and the guidewire 122. In some embodiments, a user can decouple the inflatable member 114 from the coupling member 124 by pushing the inflatable member 114 along the coupling member 124 toward an end of the coupling member 124 such that the coupling member 124 is translated through the opening created by the needle 130 while in a straight or non-straight configuration.

In some embodiments, the coupling member 124 can be configured to pierce the inflatable member 114 such that the coupling member 124 can be inserted into and/or through the inflatable member 114. In some embodiments, the system 100 can optionally include a needle 130 having a first end 131, a second end 133, and defining a lumen 135. The first end 131 can have any suitable shape configured to pierce and create access to the inflatable member 114. For example, the first end 131 can have a sharpened tip that can be tapered. The lumen 135 can be sized such that the coupling member 124 of the guidewire assembly 120 can be translated through the second end 133, through the lumen 135, and through the first end 131 of the needle 130. In some embodiments, the needle 130 can be inserted through an anterior neck and tracheal wall of the patient and through a sidewall of the inflatable member 114. The coupling member 124 and a portion of the guidewire 122 can then be translated through the lumen 135 of the needle 130 such that at least one of the coupling member 124 and a portion of the guidewire 122 is at least partially disposed within inflatable member 114. The needle 130 can then be removed from the inflatable member 114 via translating the needle 130 along the guidewire 122.

The optional barrier member 195 may be a portion of the inflation assembly 110 that is more resistant to puncturing or tearing (e.g., by a needle) than the inflatable member 114 or a portion of the inflatable member 114. In some implementations, the barrier member 195 may be arranged in any suitable location relative to a portion of the inflatable member 114 that is intended to be pierced in which the barrier member can prevent a needle from passing through the inflatable member 114 and puncturing a posterior tracheal wall of the patient. In some implementations, the barrier member 195 may form a portion of a sidewall of the inflatable member 114. In some implementations, the barrier member 195 may be disposed on and/or coupled to an outer surface of the inflatable member 114. In some implementations, the barrier member 195 may be disposed within the inflatable member 114. For example, the barrier member 195 may be disposed on or coupled to an inner surface of the inflatable member 114 (e.g., opposite a surface of the inflatable member 114 that is intended to be pierced by the needle 130). In some implementations, the barrier member 195 may be disposed on or coupled to the elongated tube 112. In some implementations, the barrier member 195 may be disposed at a location between the elongated tube 112 and an inner surface of the inflatable member 114.

The barrier member 195 may have any suitable shape. In some implementations, the barrier member 195 may have a shape corresponding to a shape of an inner surface or an outer surface of the inflatable member 114 and/or a plane passing through the inflatable member 114. For example, the barrier member 195 may have an ovular profile, a circular profile, or a rectangular profile.

In some implementations, the barrier member 195 may be sufficiently resistant to piercing and/or tearing such that, if a needle (e.g., the needle 130) applies a greater force to the barrier member 195 than a magnetic attraction force applied by the external magnetic assembly 140 on the magnetic member 115 of the inflation assembly 110 (e.g., through the skin and tracheal wall of the patient), the needle will urge the barrier member 195 toward the posterior tracheal wall of the patient and thus urge the magnetic member 115 away from the anterior tracheal wall of the patient, rather than the needle piercing the barrier member 195. Upon removal or reduction of the force of the needle on the barrier member 195 in the posterior direction, the magnetic member 115 may be urged again toward the anterior tracheal wall due to the magnetic attraction of the external magnetic assembly 140.

In some implementations, the barrier member 195 may be arranged relative to the magnetic member 115 such that, when the magnetic member 115 is urged toward the external magnetic assembly 140, the barrier member 195 is disposed opposite the magnetic member 115 from the external magnetic assembly 140 (e.g., between the magnetic member 115 and the posterior tracheal wall of the patient). In some implementations, the barrier member 195 may include one or more magnetic elements. The one or more magnetic elements may have a polarity relative to the magnetic member 115 and/or the external magnetic assembly 140 such that the barrier member 195 is repelled by the magnetic member 115 and/or the external magnetic assembly 140 such that the barrier member 195 will be urged toward the posterior tracheal wall and away from the anterior tracheal wall of the patient. The barrier member 195 may be coupled or disposed relative to the portion of the inflatable member 114 that is intended to be pierced such that, when the barrier member 195 is urged toward the posterior tracheal wall and away from the anterior tracheal wall of the patient (e.g., via magnetic interaction with the external magnetic assembly 140 and/or via the magnetic interaction of the external magnetic assembly 140 with the magnetic member 1150), the portion of the inflatable member 114 that is intended to be pierced is disposed near or adjacent to the anterior tracheal wall. Thus, the barrier member 195 may be disposed between an interior of the inflatable member 114 and the posterior tracheal wall such that, when a needle 130 is translated into the interior of the inflatable member 114, the barrier member 195 may prevent the needle 130 from being extended into contact with the posterior tracheal wall because further translation of the needle 130 may translate the needle 130 into contact with the barrier member 195.

In some implementations, the barrier member 195 may have increased echogenicity such that the barrier member 195 may be more easily visualized via ultrasound than other portions of the inflation assembly 110 (e.g., the inflatable member 114 and/or the interior of the inflatable member 114) and/or the surrounding portion of the patient. Due to the increased echogenicity, a user may be able to identify the location of the barrier member 195 via ultrasound imaging and discontinue translating the needle 130 prior to the needle reaching the barrier member 195 or prior to the needle passing the barrier member 195 such that the needle 130 may be prevented from advancing too far relative to the inflatable member 114 and/or the trachea of the patient and damaging the posterior tracheal wall of the patient.

In some implementations, the barrier member 195 may be formed of any suitable material with increased resistance to piercing by a needle used to pierce the tissue of a patient (e.g., the needle 130) and/or increased echogenicity. For example, the barrier member 195 may be formed of a polymer or metallic composite. In some implementations, the barrier member 195 may include a thickened or strengthened portion of the sidewall of the inflatable member 114.

In some embodiments, the inflatable member 114 can be filled and/or inflated with a fluid (e.g., a liquid or a gaseous fluid) after being disposed in the upper trachea of the patient. For example, the inflatable member 114 can be filled and/or inflated with a fluid and/or contrast medium such that the inflatable member 114 defines an echogenic space detectable using ultrasound imaging. Inflating the inflatable member 114 can also increase the surface tension of the sidewall of the inflatable member such that the needle 130 and/or the guidewire 122 can more easily pierce the sidewall. Further, inflation of the inflatable member 114 can create a larger interior space within which the coupling member 124 can expand and/or be disposed. Inflation of the inflatable member 114 can also increase the target size of the inflatable member for visualization and targeting of the inflatable member 114 with the needle 130 and/or coupling member 124.

The tubular member 150 can have a first end 151 and a second end 153 opposite the first end 151. The tubular member 150 can define a lumen extending from the first end 151 to the second end 153. In some embodiments, the tubular member 150 can include an inflatable member 152 configured to extend from an outer surface of the tubular member 150 near the first end 151. The inflatable member 152 can be configured to seal against the inner surface of a trachea of a patient and/or stabilize the tubular member 150 within the trachea of the patient. The second end 153 of the tubular member 150 can be configured to be coupled to a source of ventilation such that when the first end 151 of the tubular member 150 is disposed within a trachea of a patient, the patient can be ventilated via the tubular member 150. In some embodiments, the tubular member 150 can be configured to be disposed within a trachea of a patient via translating the first end 151 of the tubular member 150 through a nasal or oral orifice of the patient. In some embodiments, the tubular member 150 can be an endotracheal tube (“ETT”). The tubular member 150 can be configured to receive at least a portion of the inflation assembly 110 within the lumen of the tubular member 150 such that the inflation assembly 110 can be translated relative to the first end 151 of the tubular member 150. In some embodiments, the tubular member 150 and the inflation assembly 110 are configured such that, when the inflation assembly 110 is disposed within the lumen of the tubular member 150 and translated relative to the tubular member 150, the tubular member 150 can continue ventilating the patient (e.g., providing air to the patient’s lungs via the trachea of the patient). For example, in some embodiments, the outermost diameter or lateral extent of the inflation assembly 110 can be equal to or less than 50% of the inner diameter of the tubular member 150. In some embodiments, the inflation assembly 110 can be disposed in-line with the tubular member 150 such that the ventilator circuit is not broken and airway pressures through the trachea can be maintained.

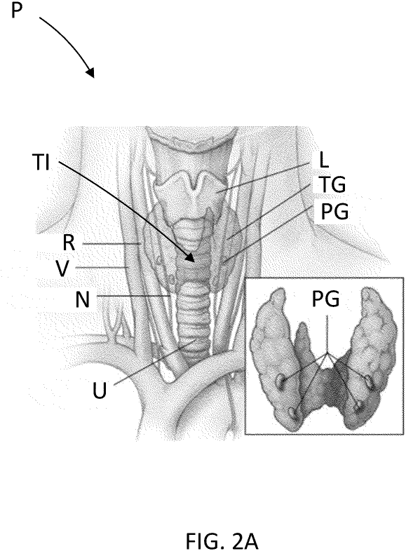

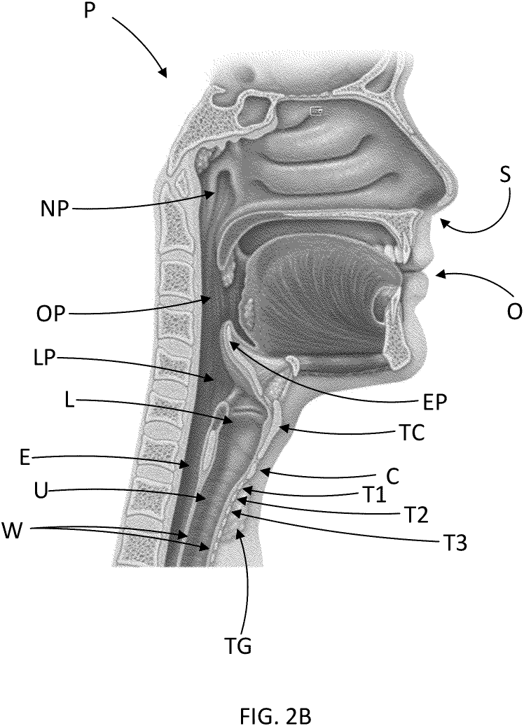

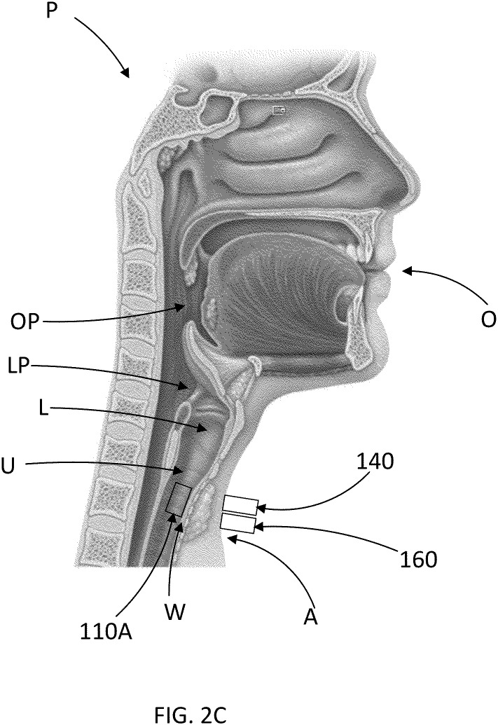

FIGS. 2A and 2B are schematic illustrations of an anterior and a cross-sectional side view of a portion of a patient P. As shown in FIGS. 2A and 2B, the patient P has an oral orifice O and a nasal orifice S. An end of a tubular member, such as the first end 151 of the tubular member 150, can be inserted through either the oral orifice O or the nasal orifice S and translated to a trachea W of the patient P. For example, the tubular member 150 can be inserted through the nasal orifice S and translated through the nasopharynx NP, the oropharynx O, passed the epiglottis EP, through the laryngopharynx LP avoiding the esophagus E, through the larynx L, passed the thyroid cartilage TC, through the cricoid ring C, and into the upper trachea U. The end of the tubular member can also be inserted through the oral orifice O and translated to the trachea W via the oropharynx OP.

The ultrasound probe 160 can be any suitable ultrasound probe configured for visualization of the inflatable member 114 within the patient and any intervening patient structure between the skin of the patient and the inflatable member 114. For example, the ultrasound probe 160 may be used to visualize any intervening patient tissue or patient structures such as a wall of the trachea W, cartilage such as thyroid cartilage TC, blood vessels such as arteries R and/or veins V, nerves such as the laryngeal nerve N, the thyroid gland TG, a portion of the thyroid gland TG such as the thyroid isthmus TI, a parathyroid gland PG, and/or any other structures or tissue that may be disposed between the inflatable member 114 and the skin of the patient). The external magnetic assembly 140 can be any suitable external magnetic assembly configured to urge the magnetic member 115 of the inflation assembly 110 (e.g., via magnetic attraction) toward the external magnetic assembly through patient tissue (e.g., through the skin and tracheal wall of the patient). As shown in FIG. 2C, the ultrasound probe 160 can be used to identify a location of a portion 110A of the inflation assembly 110 (e.g., a portion of the inflation assembly 110 including the inflatable member 114 and the magnetic member 115) within the patient P relative to other tissue or structures of the patient P. For example, as shown in FIG. 2C, with the inflation assembly 110 disposed within the patient (e.g., within the upper trachea U of the patient with the elongated tube 112 (not shown) extending from the portion 110A, through the larynx L, the laryngopharynx LP, the oropharynx OP, and out of the oral orifice O), the external magnetic assembly 140 can be coupled to an external surface of the patient (e.g., the anterior surface A of the skin of the patient’s neck) such that the magnetic member 115 (not shown in FIG. 2C) of the portion 110A of the inflation assembly 110 is urged toward the external magnetic assembly 140 and the inflatable member 114 contacts a surface of a wall of the cavity (e.g., a surface of a wall of the upper trachea U). The inflatable member 114 and the external magnetic assembly 140 can be disposed on opposite sides of the intervening tissue and/or structures such that substantially no fluid (e.g., air) gaps are disposed between the inflatable member 114 and the external magnetic assembly 140. The surface of the wall of the cavity and the external surface can be disposed on opposite sides of at least one tissue surface of the patient. The inflatable member 114 can be visualized within the cavity. For example, the inflatable member 114 can be echogenic and visualized via the ultrasound probe 160. This technique, in which an echogenic member is urged against a surface of a wall of a body cavity, and the echogenic member and all tissue planes between the echogenic member and the external surface of the patient can be visualized by ultrasound, can be referred to as Coaptive Ultrasound (CU).

In some embodiments, the external magnetic assembly 140 can include a handle. In some embodiments, the external magnetic assembly 140 can include one magnetic element configured for magnetic interaction with the magnetic member 115. In some embodiments, the external magnetic assembly 140 can include any suitable number of magnetic elements (e.g., two magnetic elements) configured for magnetic interaction with the magnetic member 115. In some embodiments, as described above, the inflation assembly 110 can include a number of magnetic members 115 (e.g., two magnetic members), and the external magnetic assembly 140 can include a corresponding number of magnetic elements.

In some embodiments, the external magnetic assembly 140 and/or the magnetic member 115 can be formed of any suitable type of magnet. For example, the external magnetic assembly 140 and/or the magnetic member 115 can include a permanent magnet, such as a neodymium iron boron (NdFeB) magnet, a samarium cobalt (SmCo) magnet, an aluminum nickel cobalt (AlNiCo) magnet, a ceramic magnet, a ferrite magnet, and/or any other suitable rare earth magnet. In some embodiments, the external magnetic assembly 140 and/or the magnetic member 115 can include a temporary magnet. In some embodiments, the external magnetic assembly 140 and/or the magnetic member 115 can be an electromagnet, such as a solenoid. In some embodiments, the external magnetic assembly 140 and/or the magnetic member 115 can generate a magnetic field having an orientation (i.e., north (N) and south (S) poles). In other embodiments, the external magnetic assembly 140 and/or the magnetic member 115 can be formed of a ferromagnetic material that is not magnetized, i.e. does not generate its own magnetic field, but can be affected by an externally-applied magnetic field. For example, the external magnetic assembly 140 and/or the magnetic member 115 can be formed of iron or steel, and application of an external magnetic field can attract the iron toward the source of the field, applying a force to the external magnetic assembly 140 and/or the magnetic member 115.

In use, the tubular member 150 can be inserted through an orifice of a patient (e.g., a nose or mouth of a patient), through a cricoid ring of the patient, and into the upper trachea of the patient such that the first end 151 and the inflatable member 152 of the tubular member 150 are disposed within the upper trachea. For example, the inflatable member 152 of the tubular member 150 can be disposed between the second and third tracheal rings of the patient. The inflation assembly 110 can then be translated through the lumen of the tubular member 150 such that the first end 111 of the elongated tube 112 of the inflation assembly 110 extends beyond the first end 151 of the tubular member 150. In some embodiments, a user can determine that the first end 111 of the elongated tube 112 is extended a particular distance beyond the first end 151 of the tubular member 150 based on the known relative lengths of the tubular member 150 and the elongated tube 112 and/or markings on at least one of the tubular member 150 or the elongated tube 112.

The external magnetic assembly 140 can then be placed on the anterior neck of the patient such that the magnetic member 115 of the inflation assembly 110 is urged toward the external magnetic assembly 140 such that the first end 111 of the elongated tube 112 is urged into contact with an anterior wall of the upper trachea. The tubular member 150 can be translated toward the cricoid ring relative to the first end 111 of the elongated tube 112, which remains in position against the anterior wall of the upper trachea due to the magnetic attraction between the external magnetic assembly 140 and the magnetic member 115.

Fluid can then be delivered to the inflatable member 114 via the inflation lumen 116. As described above, the fluid can include a fluid and/or contrast medium such that the inflatable member 114 is detectable via imaging (e.g., ultrasound). The inflatable member 114 can then be visualized (e.g., using the ultrasound probe 160) such that the location of the inflatable member 114 can be identified. The external magnetic assembly 140 can then be moved along the skin of the anterior neck of the patient to urge the magnetic member 115 toward a desired tracheal puncture site. In some embodiments, the ultrasound probe 160 can be used to determine a tracheal puncture site between particular tracheal rings (e.g., between the first and second tracheal rings of the patient or between the second and the third tracheal rings of the patient).

While visualizing the location of the inflatable member 114 using the ultrasound probe 160, the guidewire assembly 120 can be inserted through the anterior neck of the patient and into the patient’s trachea and coupled to the inflatable member 114. For example, the needle 130 can be inserted through the anterior neck and trachea of the patient and through a sidewall of the inflatable member 114 such that the first end 131 of the needle 130 (e.g., the tip) is disposed within the inflatable member 114. During insertion of the needle 130, the ultrasound probe 160 can be used to visualize the needle 130 and any intervening patient structure between the skin of the patient and the inflatable member 114. For example, the ultrasound probe 160 can be used to identify the thyroid isthmus and proximal blood vessels of the patient in real-time during insertion of the needle 130 such that the thyroid isthmus and proximal blood vessels can be avoided. Furthermore, the ultrasound probe 160 can be used to confirm that the first end 131 of the needle 130 is disposed within the inflatable member 114. Additionally or alternatively, echogenic fluid can be aspirated from the inflatable member 114 via the needle 130 (e.g., into a syringe barrel) to verify that the first end 131 of the needle 130 is disposed within the inflatable member 114.

With the first end 131 of the needle 130 disposed within the inflatable member 114, the coupling member 124 and a portion of the guidewire 122 can be inserted through the lumen 135 of the needle 130 and translated (e.g., pushed) through the lumen 135. The coupling member 124 can then be translated out from the first end 131 of the needle 130 such that the coupling member 124 is disposed within the inflatable member 114. The needle 130 can then be withdrawn from the patient via translation of the needle 130 relative to the coupling member 124 and the guidewire 122, leaving the coupling member 124 within the inflatable member 114 and the guidewire 122 extending through a wall of the inflatable member 114. Additionally, the external magnetic assembly 140 can be removed from the patient such that the magnetic member 115 is no longer urged (e.g., via magnetic attraction) toward the anterior tracheal wall. Additionally, the inflatable member 114 can be deflated.

With the guidewire assembly 120 extending through the anterior neck of the patient and coupled to the inflatable member 114, any suitable percutaneous tracheostomy procedure can be performed using the guidewire assembly 120 and the tract through the patient’s anterior neck to the patient’s trachea through which the guidewire assembly 120 is disposed. For example, the elongated tube 112 can be translated through the tubular member 150 such that the first end 111 of the elongated tube 112 moves toward the lower trachea and/or the lungs of the patient. Thus, the coupling member 124 and the guidewire 122 of the guidewire assembly 120 are translated toward the lower trachea and/or the lungs of the patient. External dilatation can then be performed via, for example, the Ciaglia technique or the Griggs technique. In some embodiments, the guidewire 122 will need to be advanced through the puncture site in the patient’s neck as the elongated tube 112 is translated to prevent the guidewire assembly 120 from decoupling with the inflatable member 114.

As another example, a translaryngeal tracheostomy (or Fantoni technique) can be performed using the guidewire assembly 120. For example, the tubular member 150 and the elongated tube 112 can be translated such that the first end 111 of the elongated tube 112 moves through an orifice (e.g., nasal or oral) of the patient. Thus, the coupling member 124 and the guidewire 122 of the guidewire assembly 120 are translated toward the cricoid ring and through the orifice of the patient. In some embodiments, the tubular member 150 can be withdrawn from the patient via the orifice of the patient prior to withdrawing the elongated tube 112. A tracheostomy tube can then be threaded over the guidewire such that the tracheostomy tube can be translated through the patient’s mouth, through the cricoid ring, into the upper trachea, and into engagement with the tract through the patient’s tracheal wall and anterior neck.

In some embodiments, to verify that the coupling member 124 of the guidewire is disposed within the trachea of the patient and engaged with the inflatable member 114, the elongated tube 112 can be translated through the tubular member 150 such that the first end 111 of the elongated tube 112 moves through an orifice (e.g., nasal or oral) of the patient. Thus, the coupling member 124 and the guidewire 122 of the guidewire assembly 120 are translated toward the cricoid ring and through or near the orifice of the patient. The engagement between the coupling member 124 and the inflatable member 114 can then be verified. After the verification, the guidewire 122 can be pulled through the tract in the anterior neck of the patient such that the coupling member 124 pulls the inflatable member 114 and the elongated tube 112 into the upper trachea. Any suitable percutaneous tracheostomy can then be performed using the guidewire assembly 120.

In some embodiments, the inflatable member 114 can define passageways (not shown) (also referred to as “vents”) extending from a first end to a second end of the inflatable member 114 such that fluid (e.g., air) can travel through the passageways when the inflatable member 114 is in the expanded configuration. For example, in some embodiments, when the inflatable member 114 is disposed in the trachea in the expanded configuration, each passageway can be partially bounded by the inflatable member 114 and partially bounded by the patient’s tracheal wall. Thus, air can flow between a portion of the trachea on a first side of the inflatable member 114 (e.g., a region between the cricoid ring and the inflatable member 114) and a portion of the trachea on a second side of the inflatable member 114 (e.g., a region between the inflatable member 114 and the lungs of the patient) through the passageways when the inflatable member 114 is in the expanded configuration. In some embodiments, the inflatable member 114 can define the entire outer boundary of one or more of the passageways. The inflatable member 114 can be shaped to define any suitable number of passageways in the expanded configuration (e.g., one, two three, four, or more passageways). The passageways can be defined in any suitable location of the inflatable member 114. In some embodiments, the inflatable member 114 can define a suitable number of passageways having a suitable size (e.g., having a suitable combined cross-sectional area) such that a patient can be properly ventilated (e.g., freely or via a ventilator) through the passageways when the inflatable member 114 is disposed in a trachea of the patient and the outer surface of the inflatable member 114 contacts the inner wall of the trachea of the patient. For example, the outer surface of the inflatable member 114 can contact the inner wall of the trachea of the patient in an uninterrupted manner such that air cannot flow between the outermost surface of the inflatable member 114 and the tracheal wall or the rate that air can flow between the outermost surface and the tracheal wall is insufficient for proper ventilation and oxygenation. In some embodiments, the combined cross-sectional area of the passageways can be sufficiently large to allow for between about 40 and about 75 liters per minute of airflow through the passageways. Thus, with the inflatable member 114 deployed in the trachea, the patient can receive closed and continuous ventilation via the passageways, including during ultrasound visualization (e.g., via Coaptive Ultrasound) of the inflatable member 114 and/or tissue between the inflatable member 114 and an ultrasound probe.

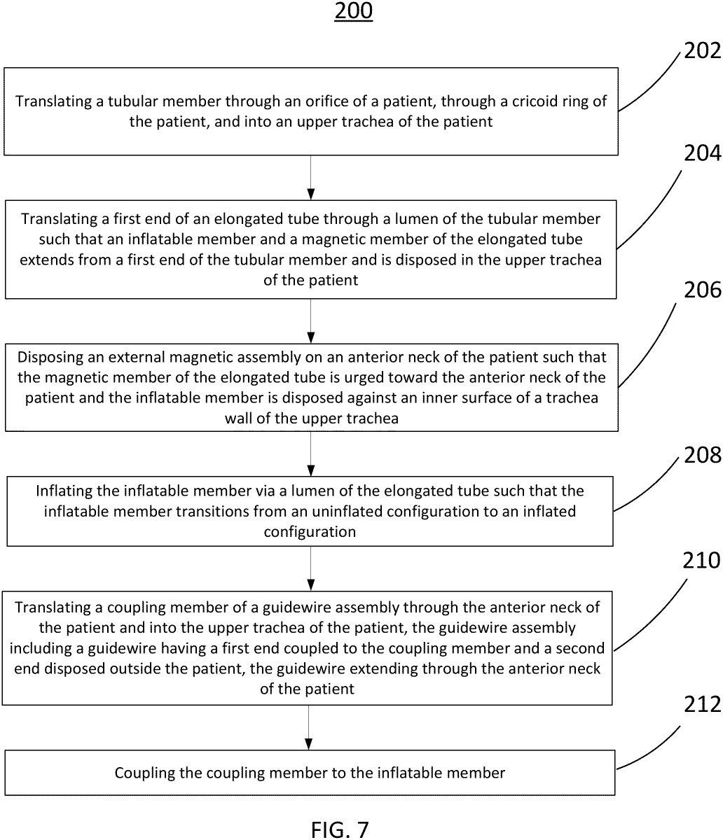

FIG. 7 is a flow chart of a method 200, according to an embodiment. The method 200 can be implemented using any of the systems or devices described herein, such as the system 100 described above. The method 200 includes translating 202 a tubular member through an orifice of a patient, through a cricoid ring of the patient, and into an upper trachea of the patient. A first end of an elongated tube can be translated 204 through a lumen of the tubular member such that an inflatable member and a magnetic member of the elongated tube extends from a first end of the tubular member and is disposed in the upper trachea of the patient. An external magnetic assembly can be disposed 206 on an anterior neck of the patient such that the magnetic member of the elongated tube is urged toward the anterior neck of the patient and the inflatable member is disposed against an anterior inner surface of the upper trachea. The inflatable member can then be inflated 208 via a lumen of the elongated tube such that the inflatable member transitions from an uninflated configuration to an inflated configuration. A coupling member of a guidewire assembly can be translated 210 through the anterior neck of the patient and into the upper trachea of the patient. The guidewire assembly can include a guidewire having a first end coupled to the coupling member and a second end disposed outside the patient, the guidewire extending through the anterior neck of the patient. The coupling member can be coupled 212 to the inflatable member.

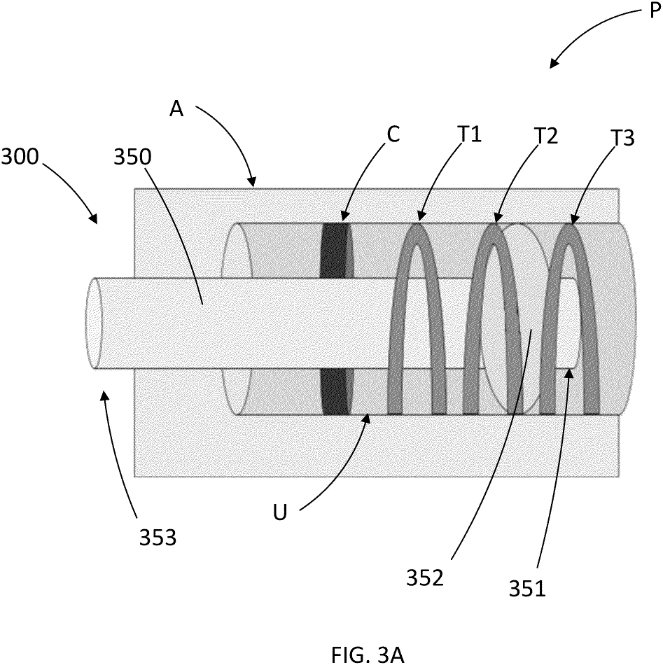

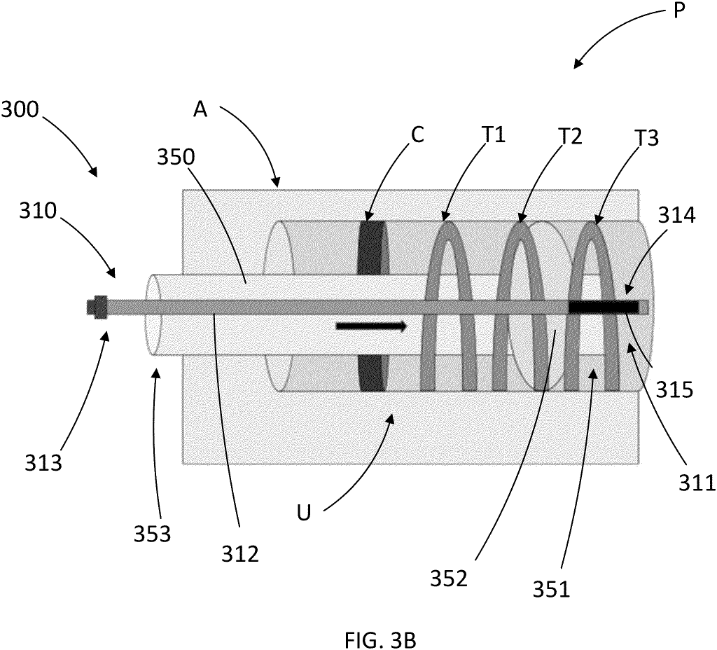

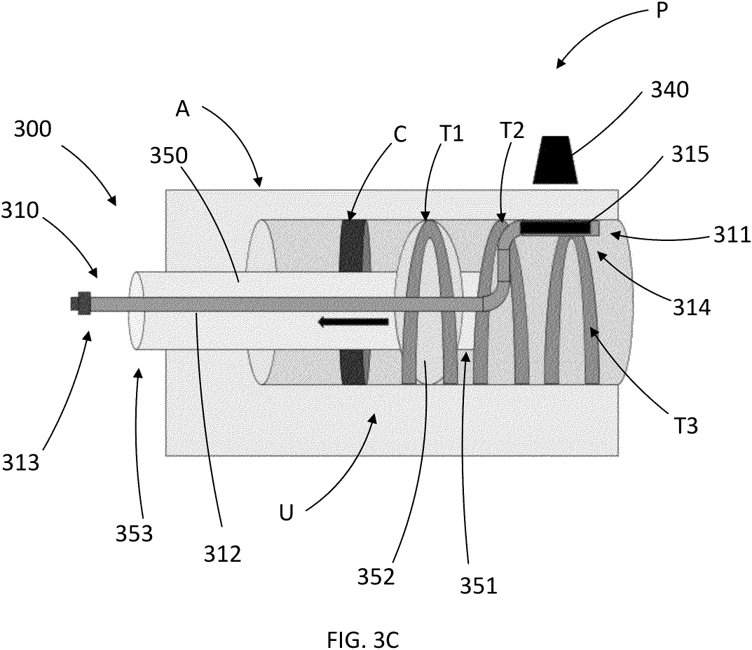

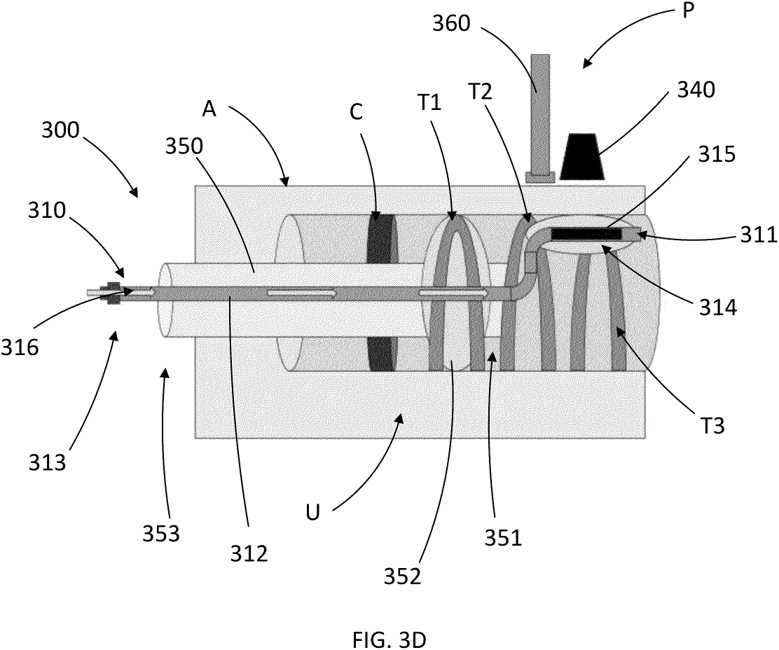

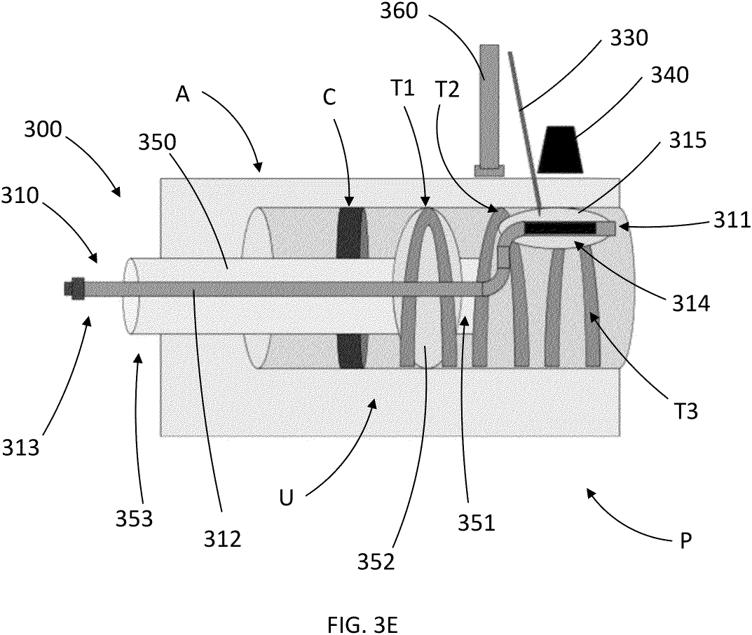

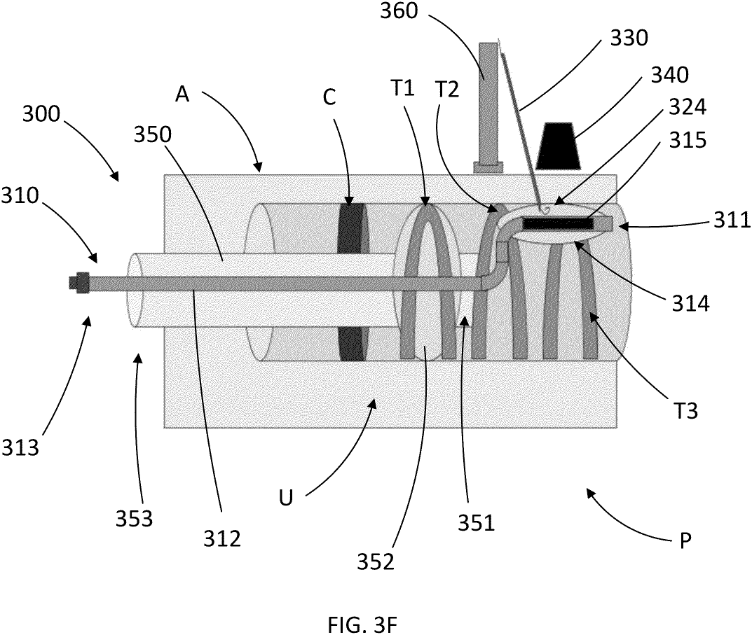

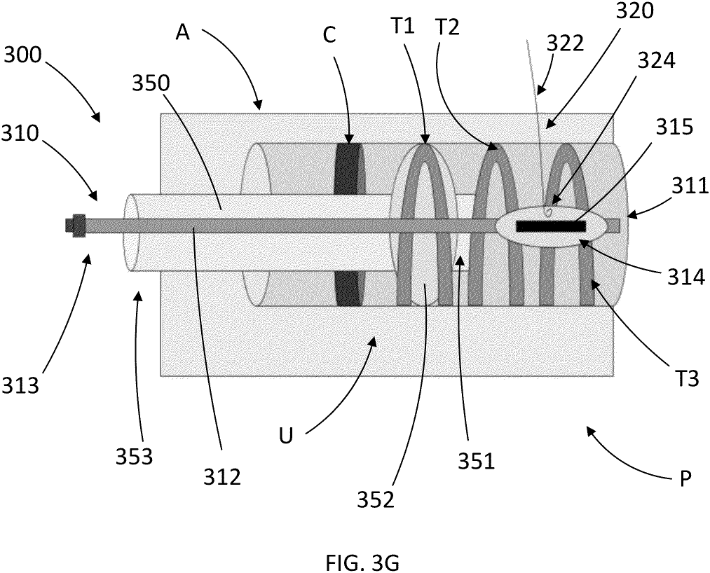

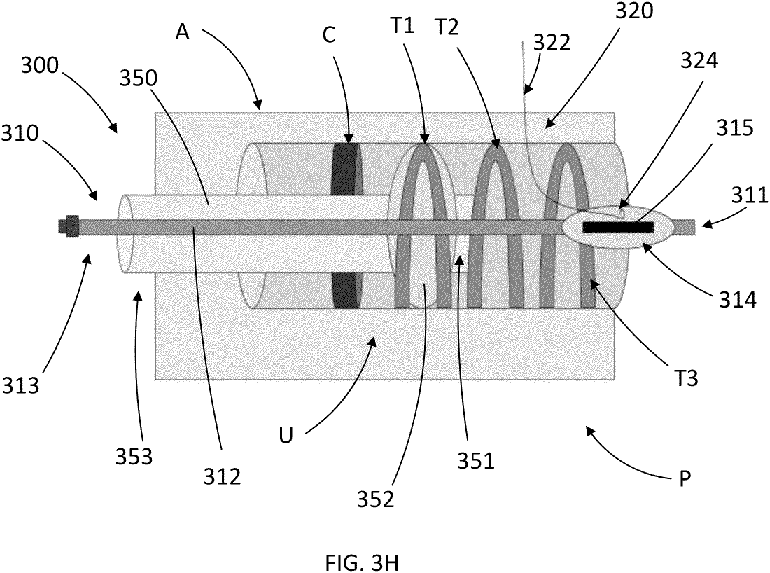

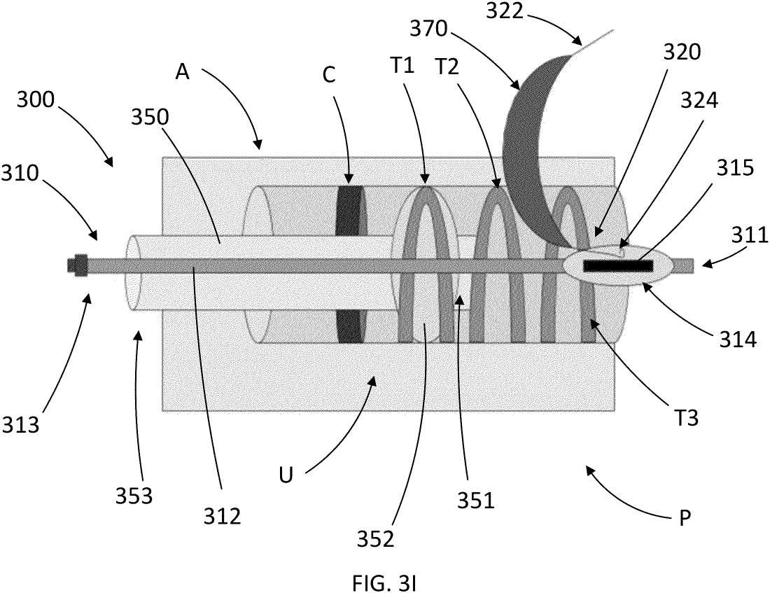

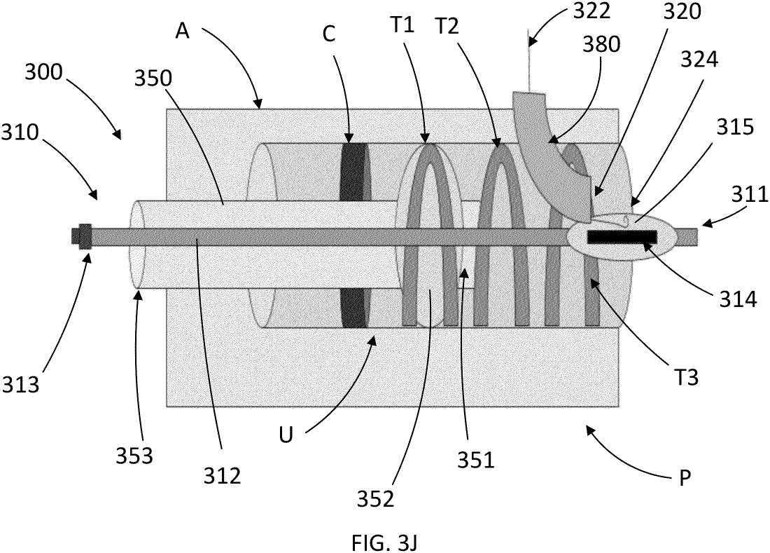

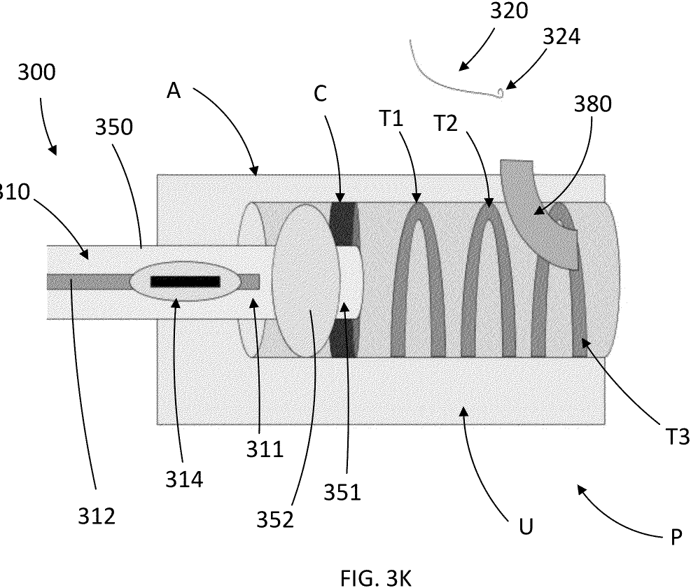

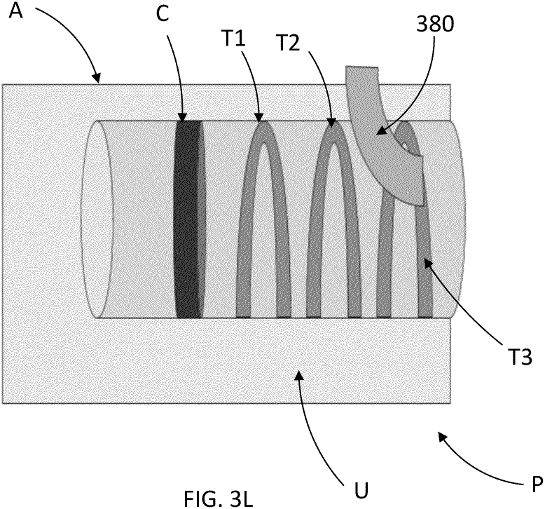

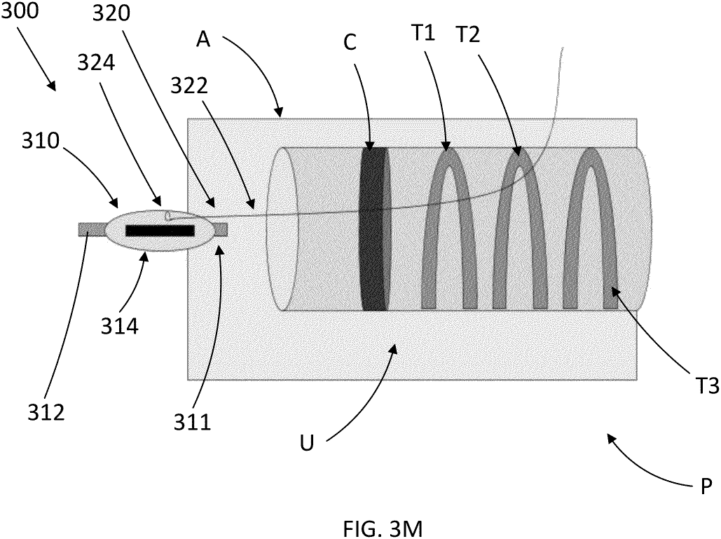

FIGS. 3A-3M are schematic illustrations of a system 300 in various stages of operation. The system 300 can be the same or similar in structure and/or function to any of the systems or devices described herein, such as the system 100 described above. For example, the system 300 includes an inflation assembly 310, a guidewire assembly 320, and a tubular member 350. The system 300 also includes an external magnetic assembly 340 and an ultrasound probe 360. The inflation assembly 310 can include an elongated tube 312, an inflatable member 314, and a magnetic member 315. The elongated tube 312 can have a first end 311 and a second end 313. In some embodiments, the elongated tube 312 can have a length sufficient to extend from at least an oral or nasal orifice of a patient to the trachea of the patient. The inflatable member 314 and the magnetic member 315 can be coupled to the elongated tube 312 near the first end 311 of the elongated tube 312. The inflation assembly 310 includes an inflation lumen 316 defined by the elongated tube 312 and in fluid communication with the inflatable member 314. The guidewire assembly 320 can include a guidewire 322 having a first end 321 and a second end 323 and a coupling member 324 disposed at the first end 321 of the guidewire 322. The tubular member 350 can have a first end 351 and a second end 353 opposite the first end 351. The tubular member 350 can define a lumen extending from the first end 351 to the second end 353 and can include an inflatable member 352 configured to transition from an uninflated configuration to an inflated configuration in which the inflatable member 352 extends from an outer surface of the tubular member 350 near the first end 351 and couples to the inner surface of the tracheal wall of the patient.

As shown in FIG. 3A, the tubular member 350 can be inserted through an orifice (not shown) of a patient P (e.g., a nose or mouth of a patient), through a cricoid ring C of the patient P, and into the upper trachea U of the patient P such that the first end 351 and the inflatable member 352 of the tubular member 350 are disposed within the upper trachea U. The inflatable member 352 can be transitioned to the inflated configuration after the inflatable member 352 has been inserted through the cricoid ring C of the patient P such that the inflatable member 352 can secure the first end 351 within the upper trachea U and/or such that the first end 351 of the tubular member 350 can be stabilized (e.g., resistant to axial movement) relative to the inner surface of the tracheal wall. For example, the inflatable member 352 of the tubular member 350 can be disposed between the second tracheal ring T2 and third tracheal ring T3 of the patient P or between the first tracheal ring T1 and the second tracheal ring T2 of the patient P. The inflatable member 352 can have any suitable shape. For example, the inflatable member 352 can have a circular profile in the inflated configuration such that a central axis of the first end 351 of the tubular member 350 can be coaxial with a central axis of the upper trachea U.

As shown in FIG. 3B, the inflation assembly 310 can then be translated through the lumen of the tubular member 350 such that the first end 311 of the elongated tube 312 of the inflation assembly 310 extends beyond the first end 351 of the tubular member 350. In some embodiments, a user can determine that the first end 311 of the elongated tube 312 is extended a particular distance beyond the first end 351 of the tubular member 350 based on the known relative lengths of the tubular member 350 and the elongated tube 312 and/or markings on at least one of the tubular member 350 or the elongated tube 312.

As shown in FIG. 3C, the external magnetic assembly 340 can then be placed on the anterior neck A of the patient such that the magnetic member 315 of the inflation assembly 310 is urged toward the external magnetic assembly 340 such that the first end 311 of the elongated tube 312 is urged into contact with an anterior wall of the upper trachea U. The tubular member 350 can be translated toward the cricoid ring C relative to the first end 311 of the elongated tube 312, which remains in position against the anterior wall of the upper trachea U due to the magnetic attraction between the external magnetic assembly 340 and the magnetic member 315. For example, in some implementations, the first end 351 of the tubular member 350 can be translated toward the cricoid ring C with the inflatable member 352 inflated by pulling the inflatable member 352 along the inner surface of the wall of the upper trachea U. In some implementations, the inflatable member 352 of the tubular member 350 can be partially or fully deflated prior to being translated toward the cricoid ring C, and then reinflated after being translated. In some implementations, the tubular member 350 can remain in its initial position relative to the cricoid ring C rather than translating the first end 351 of the tubular member 350 toward the cricoid ring C before proceeding (e.g., the inflatable member 352 can be initially inflated between the cricoid ring C and the first tracheal ring T1).

As shown in FIG. 3D, fluid can then be delivered to the inflatable member 314 via the inflation lumen 316 (e.g., via an inflation port coupled to the second end 313 of the elongated tube 312). As described above, the fluid can include a fluid and/or contrast medium such that the inflatable member 314 is detectable via imaging (e.g., ultrasound). An ultrasound probe 360 can then be applied to the anterior neck A of the patient P such that the inflatable member 314 and any intervening tissue or other structure between the inflatable member and the surface of the anterior neck A can be visualized and the location of the inflatable member 314 can be identified. The external magnetic assembly 340 can then be moved along the skin of the anterior neck A of the patient to urge the magnetic member 315 toward a desired tracheal puncture site. In some embodiments, the ultrasound probe 360 can be used to determine a tracheal puncture site between particular tracheal rings (e.g., between the first and second tracheal rings T1, T2 of the patient P or between the second and the third tracheal rings T2, T3 of the patient P).

As shown in FIG. 3E, while visualizing the location of the inflatable member 314 using the ultrasound probe 360, the needle 330 can be inserted through the anterior neck A of the patient P and into the patient’s upper trachea U. The needle 330 can be further translated such that the needle 330 is inserted through a sidewall of the inflatable member 314 such that the first end 331 of the needle 330 (e.g., the tip) is disposed within the inflatable member 314. During insertion of the needle 330, the ultrasound probe 360 can be used to visualize the needle 330 and any intervening patient structure between the skin of the patient and the inflatable member 314. For example, the ultrasound probe 360 can be used to identify the thyroid isthmus and proximal blood vessels of the patient in real-time during insertion of the needle 330 such that the thyroid isthmus and proximal blood vessels can be avoided. Furthermore, the ultrasound probe 360 can be used to confirm that the first end 331 of the needle 330 is disposed within the inflatable member 314. Additionally or alternatively, echogenic fluid can be aspirated from the inflatable member 314 via the needle 330 (e.g., into a syringe barrel) to verify that the first end 331 of the needle 330 is disposed within the inflatable member 314.

As shown in FIG. 3F, with the first end 331 of the needle disposed within the inflatable member 314, the coupling member 324 and a portion of the guidewire 322 can be inserted through the lumen 335 of the needle 330 and translated (e.g., pushed) through the lumen 335. The coupling member 324 can then be translated out from the first end 331 of the needle 330 such that the coupling member 324 is disposed within the inflatable member 314.

As shown in FIG. 3G, the needle 330 can then be withdrawn from the patient via translation of the needle 330 relative to the coupling member 324 and the guidewire 322, leaving the coupling member 324 within the inflatable member 314 and the guidewire 322 extending through a wall of the inflatable member 314. Additionally, the external magnetic assembly 340 can be removed from the patient such that the magnetic member 315 is no longer urged (e.g., via magnetic attraction) toward the anterior tracheal wall. The inflatable member 314 can be deflated.

With the guidewire assembly 320 extending through the anterior neck of the patient and coupled to the inflatable member 314, any suitable percutaneous tracheostomy procedure can be performed using the guidewire assembly 320 and the tract through the patient’s anterior neck to the patient’s trachea through which the guidewire assembly 320 is disposed. For example, it may be desired to translate the coupling member 324 of the guidewire assembly 320 through the tubular member 350 and out of the patient’s mouth using the inflation assembly 310 (e.g., for use of the guidewire 322 for airway control and reintroduction of an ETT in the event that the tubular member 350 is accidentally removed from the trachea). As shown in FIG. 3H, the elongated tube 312 can be translated through the tubular member 350 such that the first end 311 of the elongated tube 312 moves toward the lower trachea and/or the lungs of the patient P. Thus, the coupling member 324 and the guidewire 322 of the guidewire assembly 320 are translated toward the lower trachea and/or the lungs of the patient. External dilatation can then be performed via, for example, the Ciaglia technique or the Griggs technique. In some embodiments, the guidewire 322 will need to be advanced through the puncture site in the patient’s neck as the elongated tube 312 is translated to prevent the guidewire assembly 320 from decoupling with the inflatable member 314. In some embodiments, the guidewire 322 may be pushed such that the inflatable member 314 is translated.

As an example of the Ciaglia technique, FIG. 3I shows that a dilator 370 can be advanced over the guidewire 322 to externally dilate the tract through the anterior neck A to the upper trachea U. As shown in FIG. 3J, after external dilation, a tracheostomy tube 380 can be advanced over the guidewire 322 such that a first end of the tracheostomy tube 380 is disposed within the upper trachea U and a second end of the tracheostomy tube 380 is disposed outside of the patient P (e.g., extending from the anterior neck A of the patient P). As shown in FIG. 3K, the guidewire assembly 320 can be uncoupled from the inflatable member 314. For example, a force sufficient to cause the coupling member 324 to decouple from the inflatable member 314 can be applied to the guidewire assembly 320 in a direction away from the patient P. The guidewire assembly 320 can then be translated through the tracheostomy tube 380 and removed from the patient P. The tracheostomy tube 380 can be coupled to a ventilator (not shown) such that the ventilator can push air into the lungs (not shown) of the patient P and draw air from the lungs of the patient P via the tracheostomy tube 380. After testing the ventilator and tracheostomy tube 380 combination to ensure that the ventilator is properly pushing air into the lungs of the patient P and drawing air from the lungs of the patient P via the tracheostomy tube 380, the inflation assembly 310 and tubular member 350 can be removed from the patient P. For example, the elongated tube 312 can be translated proximally through the tubular member 350, and the elongated tube 312 and the tubular member 350 can be sequentially or simultaneously removed from the patient P via proximal translation through an orifice of the patient P through which the tubular member 350 was inserted into the patient P. For example, the inflatable member 352 can be fully or partially deflated and the tubular member 350 can be proximally translated. As shown in FIG. 3L, the tracheostomy tube 380 can remain in place relative to the anterior neck A and upper trachea U of the patient, providing fluid flow access to the lungs of the patient.

As another example, a translaryngeal tracheostomy or Fantoni technique can be performed using the guidewire assembly 320. For example, as shown in FIG. 3M, the tubular member 350 can be withdrawn from the patient P through an orifice (e.g., nasal or oral) of the patient P. The elongated tube 312 can be translated such that the first end 311 of the elongated tube 312 moves through the orifice of the patient P. Thus, the coupling member 324 and the guidewire 322 of the guidewire assembly 320 are translated through the cricoid ring C and out through the orifice of the patient P. A tracheostomy tube can then be threaded over the guidewire such that the tracheostomy tube can be translated through the patient’s mouth, through the cricoid ring C, into the upper trachea U, and into engagement with the tract through the patient’s tracheal wall and anterior neck A.

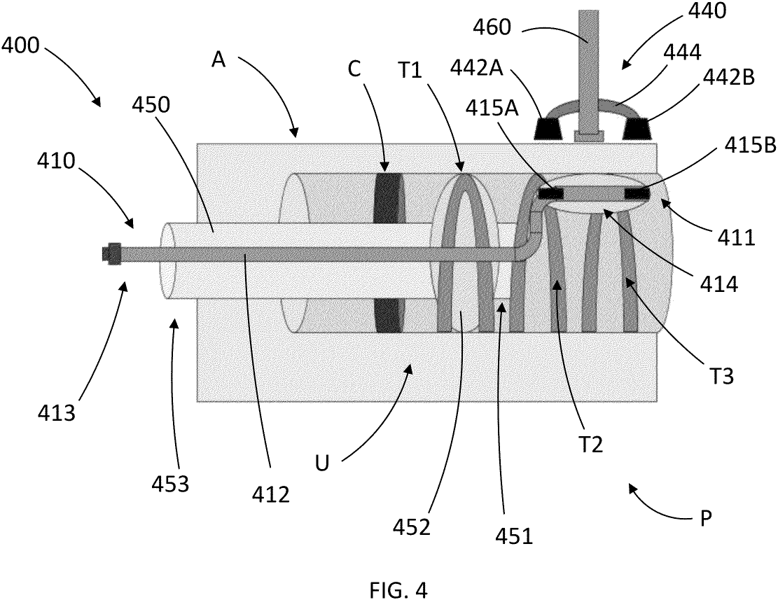

In some embodiments, the external magnetic assembly and the inflation assembly can include any suitable number of magnetic elements configured for magnetic interaction through tissue of the patient. For example, FIG. 4 is a schematic illustration of a system 400. The system 400 can be the same or similar in structure and/or function to any of the systems or devices described herein, such as the system 100 and/or the system 300 described above. For example, the system 400 includes an inflation assembly 410, a guidewire assembly 420, and a tubular member 450. The system 400 also includes an external magnetic assembly 440 and an ultrasound probe 460. The inflation assembly 410 can include an elongated tube 412 and an inflatable member 414. The elongated tube 412 can have a first end 411 and a second end 413. In some embodiments, the elongated tube 412 can have a length sufficient to extend from at least an oral or nasal orifice of a patient to the trachea of the patient. The inflatable member 414 and the magnetic member 415 can be coupled to the elongated tube 412 near the first end 411 of the elongated tube 412. The inflation assembly 410 includes an inflation lumen 416 defined by the elongated tube 412 and in fluid communication with the inflatable member 414. The inflation assembly 410 can also include a first magnetic member 415A and a second magnetic member 415B. The guidewire assembly 420 can include a guidewire 422 having a first end 421 and a second end 423 and a coupling member 424 disposed at the first end 421 of the guidewire 422. The tubular member 450 can have a first end 451 and a second end 453 opposite the first end 451. The tubular member 450 can define a lumen extending from the first end 451 to the second end 453 and can include an inflatable member 452 configured to extend from an outer surface of the tubular member 450 near the first end 451 and couple to the inner surface of the tracheal wall of the patient.

The external magnetic assembly 440 can include a first magnetic element 442A, a second magnetic element 442B, and a handle 444. The first magnetic member 415A and the second magnetic member 415B of the inflation assembly 410 can be spaced along the elongated tube 412 such that the first magnetic member 415A is configured for magnetic interaction with the first magnetic element 442A and the second magnetic member 415B is configured for magnetic interaction with the second magnetic element 442B. During use of the system 400, the ultrasound probe 460 can be positioned between the first magnetic element 442A and the second magnetic element 442B to visualize the inflatable member 414 of the inflation assembly 410. This embodiment also enables more control over the orientation of the first end 411 of the elongated tube 412, because its orientation will match the orientation of the external magnet assembly 440, i.e. of the first magnetic element 422A and the second magnetic element 422B.

In some embodiments, rather than having a plurality of magnetic members and/or elements, the inflation assembly 410 can include a disc-shaped magnetic member defining a through-hole and the external magnetic assembly 440 can include a disc-shaped magnetic element defining a through-hole. The ultrasound probe 460 can be inserted through the through-hole in the disc-shaped magnetic element of the external magnetic assembly 440 and into contact with the patient P.

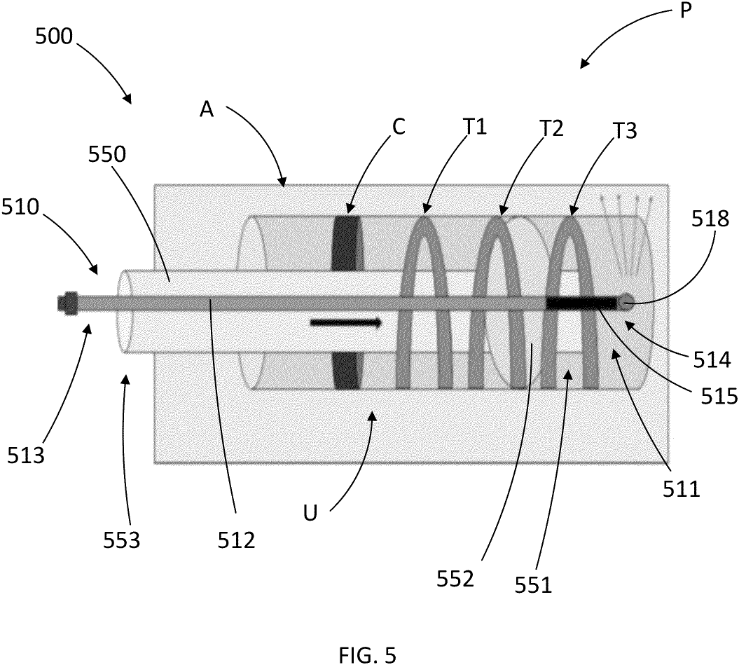

In some embodiments, as discussed above, a light source may be disposed on or near a first end of an elongated tube such that the location of the first end of the elongated tube within the patient can be identified by the location on the surface of the patient through which light is emitted. For example, FIG. 5 is a schematic illustration of a system 500. The system 500 can be the same or similar in structure and/or function to any of the systems or devices described herein, such as the system 100 and/or the system 300 described above. For example, the system 500 includes an inflation assembly 510 and a tubular member 550. The inflation assembly 510 can include an elongated tube 512 and an inflatable member 514. The elongated tube 512 can have a first end 511 and a second end 513. In some embodiments, the elongated tube 512 can have a length sufficient to extend from at least an oral or nasal orifice of a patient to the trachea of the patient. The inflatable member 514 and the magnetic member 515 can be coupled to the elongated tube 512 near the first end 511 of the elongated tube 512. The inflation assembly 510 includes an inflation lumen 516 defined by the elongated tube 512 and in fluid communication with the inflatable member 514. The tubular member 550 can have a first end 551 and a second end 553 opposite the first end 551. The tubular member 550 can define a lumen extending from the first end 551 to the second end 553 and can include an inflatable member 552 configured to extend from an outer surface of the tubular member 550 near the first end 551 and couple to the inner surface of the tracheal wall of the patient.

As shown in FIG. 5, a light source 518 may be disposed on or near the first end 511 of the elongated tube 512. The light source may produce sufficient light such that light can emit from the light source, through the tracheal wall, to the surface of the anterior neck A and be visible to a user (e.g., a clinician). Thus, the user may be able to determine the location of the first end 511 of the elongated tube 512 based, at least in part, on the location of light emitting through the anterior neck A of a patient P. The light source 518 can be, for example, a light emitting diode (LED).

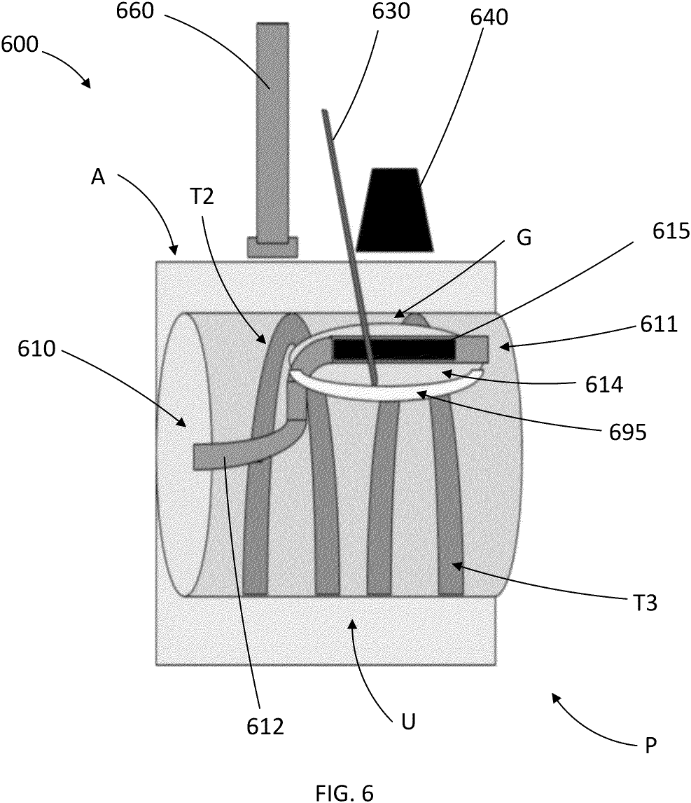

In some embodiments, as discussed above with respect to FIG. 1, an inflation assembly may include a barrier member to prevent a needle from puncturing a posterior tracheal wall of a patient. For example, FIG. 6 is a schematic illustration of a system 600. The system 600 can be the same or similar in structure and/or function to any of the systems or devices described herein, such as the system 100 and/or the system 300 described above. For example, the system 600 includes an inflation assembly 660. The system 600 also includes an external magnetic assembly 640, an ultrasound probe 660, and a needle 630. The inflation assembly 610 can include an elongated tube 612 and an inflatable member 614. The elongated tube 612 can have a first end 611 and a second end (not shown). In some embodiments, the elongated tube 612 can have a length sufficient to extend from at least an oral or nasal orifice of a patient to the trachea of the patient. The inflatable member 614 and the magnetic member 615 can be coupled to the elongated tube 612 near the first end 611 of the elongated tube 612. The inflation assembly 610 includes an inflation lumen defined by the elongated tube 612 and in fluid communication with the inflatable member 614.

As shown in FIG. 6, a barrier member 695 may be coupled to or form a portion of the sidewall of the inflatable member 614. The barrier member 695 may be the same or similar in structure and/or function as the barrier member 195 described above with respect to FIG. 1. For example, the barrier member 695 may have a shape that corresponds to a shape of the outer surface of the inflatable member 614. The barrier member 195 may be configured to be disposed between a portion of the inflatable member 614 intended to be pierced by the needle 630 and the posterior tracheal wall of the patient P. Furthermore, as shown in FIG. 6, the barrier member 695 may be sufficiently resistant to piercing and/or tearing such that, if the needle 630 applies a greater force to the barrier member 695 than a magnetic attraction force applied by the external magnetic assembly 640 on the magnetic member 615 of the inflation assembly 610 (e.g., through the anterior neck A and the tracheal wall of the patient P), the needle 630 may urge the barrier member 695 toward the posterior tracheal wall of the patient P and thus urge the magnetic member 615 away from the anterior tracheal wall of the patient P, rather than the needle 630 piercing the barrier member 695 and/or passing through the barrier member 695. Thus, a gap G may exist between the outer surface of the inflatable member 614 and the inner surface of the anterior tracheal wall of the patient P when the needle 630 is urged against the barrier member 695 while the external magnetic assembly urges the magnetic member 615 toward the anterior neck A via magnetic attraction. Upon removal or reduction of the force of the needle 630 on the barrier member 695 in the posterior direction, the magnetic member 615 may be urged again toward the anterior neck A due to the magnetic attraction of the external magnetic assembly 640.

In some implementations, the barrier member 695 can be disposed inside the inflatable member 614 and coupled to an interior surface of a sidewall of the inflatable member 614. In some implementations, the barrier member 695 can be disposed outside of the inflatable member 614 and coupled to an exterior surface of a sidewall of the inflatable member 614. In some implementations, the inflatable member 614 can be partially formed of the barrier member 695. For example, the inflatable member 614 can include a first sidewall portion and a second sidewall portion opposite the first sidewall portion. The first sidewall portion can be configured to receive the needle 630 therethrough and the second sidewall portion can be configured to be more resistant to being pierced by the needle 630 than the first sidewall portion. For example, the second sidewall portion can have a greater thickness than the first sidewall portion and/or have a greater hardness than the first sidewall portion.

Additionally, in some implementations, the barrier member 695 may have increased echogenicity such that the barrier member 695 may be more easily visualized via ultrasound than other portions of the inflation assembly 610 (e.g., the inflatable member 614 and/or the interior of the inflatable member 614) and/or the surrounding portion of the patient P. Due to the increased echogenicity, a user may be able to identify the location of the barrier member 695 via ultrasound imaging and discontinue translating the needle 630 prior to the needle reaching the barrier member 195 or prior to the needle passing the barrier member 695 such that the needle 630 may be prevented from advancing too far relative to the inflatable member 614 and/or the trachea of the patient and damaging the posterior tracheal wall of the patient.

In some implementations, the barrier member 695 may be formed of any suitable material with increased resistance to piercing by a needle used to pierce the tissue of a patient (e.g., the needle 630) and/or increased echogenicity. For example, the barrier member 695 may be formed of a polymer or metallic composite. In some implementations, the barrier member 695 may be formed a material that is the same or different from the inflatable member 614 or a remainder of the inflatable member 614. In some implementations, the barrier member 695 may include a thickened or strengthened portion of the sidewall of the inflatable member 614. The barrier member 695 may have an increased hardness (e.g., scratch hardness and/or indentation hardness) relative to the hardness of the inflatable member 614 or remainder of the inflatable member 614.

For some patients, it may be undesirable to deliver any device through the ETT. Such delivery can expose the health care provider to contagious respiratory pathogens, for example viruses such as COVID-19. Even if the patient is not known to have a contagious respiratory disease, as a precaution, for example during pandemics such as the COVID-19 pandemic, health care providers may assume that any patient is potentially infectious. For such patients or circumstances, it may be acceptable (or even desirable) to have a brief apnic period during a percutaneous tracheotomy. In that case, a guidewire delivery system may preferably be placed not inline with (i.e. through the lumen of) the ETT. Rather, the system may be delivered outside of the ETT, between the ETT and the vocal cords or folds, in what is referred to herein as a “paired approach.”

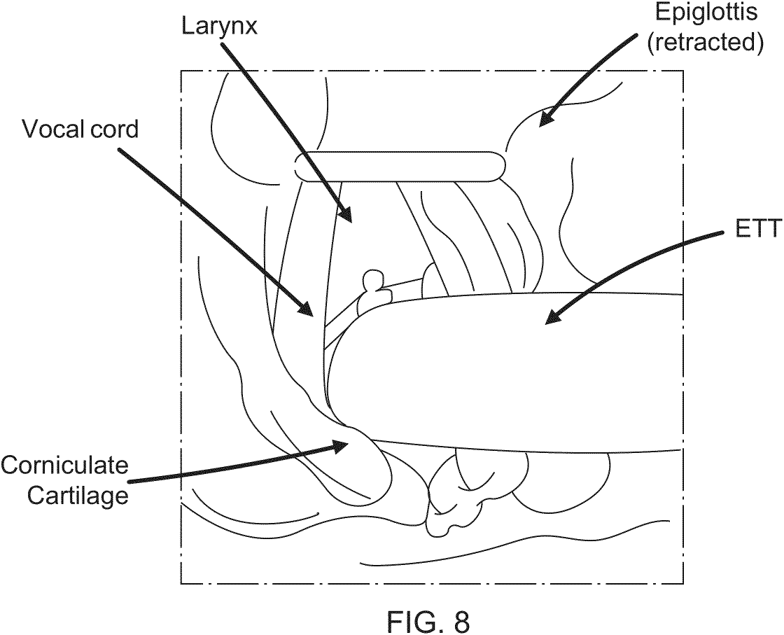

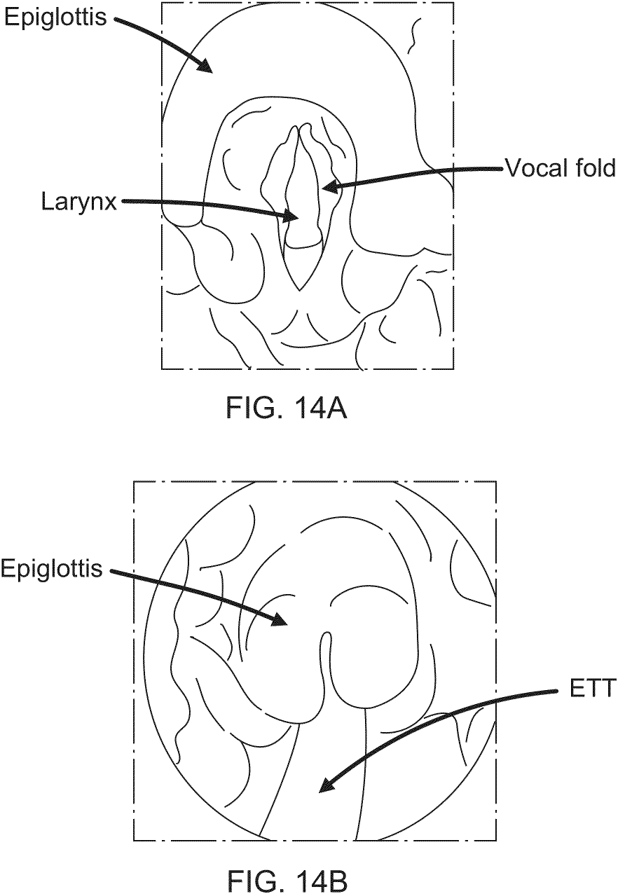

FIG. 8 is an illustration of the larynx of a patient, with the epiglottis retracted, and with an ETT shown disposed into the larynx, between the vocal cords. As can be seen in FIG. 8, because the ETT has an outer perimeter that is substantially smaller than the perimeter of the opening bounded by the vocal cords, the corniculate cartilage, and the retracted epiglottis (e.g., the glottis), there is an opening through which a device, such as a guidewire delivery system, can be delivered into the larynx. For example, FIG. 14A shows a patient having an epiglottis that is shaped and disposed in such a way relative to the vocal folds so that both an ETT and a device can be delivered into the larynx and to the trachea of the patient. FIG. 14B shows a patient having an epiglottis that is shaped and disposed in such a way relative to the vocal folds so that delivery of a device in addition to the ETT and outside of the ETT may not be feasible (e.g., due to the glottic area being too small and/or swollen). FIG. 9 is a schematic illustration of a side view of a patient’s larynx with an ETT disposed in the larynx. FIG. 10 shows a guidewire delivery system 800 delivered by a paired approach and disposed in between the ETT and the wall of the larynx, on the anterior side.

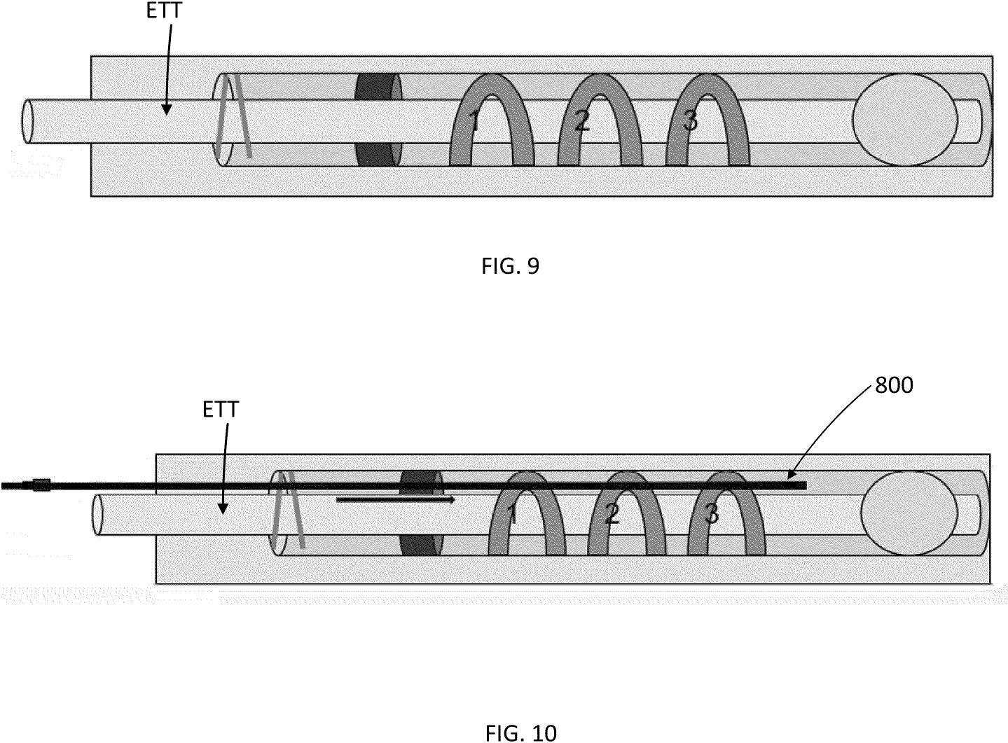

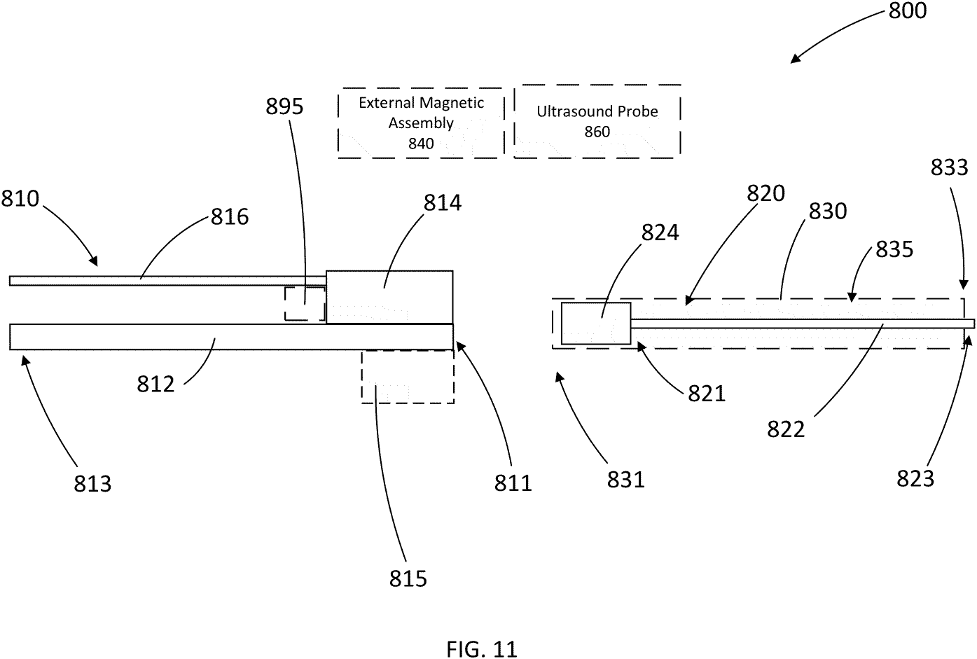

FIG. 11 is a schematic representation of a system 800. The system 800 includes an inflation assembly 810 and a guidewire assembly 820. The system 800 can optionally also include an external magnetic assembly 840 and an ultrasound probe 860. The inflation assembly 810 can include an elongated tube 812, an inflatable member 814, and optionally a magnetic member 815. The inflation assembly 810 may optionally include a barrier member 895. The elongated tube 812 can have a first end 811 and a second end 813. In some embodiments, the elongated tube 812 can have a length sufficient to extend from at least an oral or nasal orifice of a patient to the trachea of the patient. The inflatable member 814 and, optionally, the magnetic member 815, can be coupled to the elongated tube 812 at or near the first end 811 of the elongated tube 812. The inflation assembly 810 can include an inflation lumen 816 in fluid communication with the inflatable member 814. In some embodiments, the inflation lumen 816 can be disposed within and/or be defined by the elongated tube 812.

The optional magnetic member 815 can be any suitable magnetic member configured such that movement of the magnetic member 815 causes corresponding movement of the first end 811 of the elongated tube 812. The magnetic member 815 can have any suitable shape. For example, in some embodiments, the magnetic member 815 can be shaped as an elongated rectangle. In some embodiments, the magnetic member 815 can be shaped as a cylinder. In some embodiments, the magnetic member 815 may be arcuate. In some embodiments, the magnetic member 815 is coupled directly to the elongated tube 812. In some embodiments, the magnetic member 815 is disposed within the inflatable member 814 and at least partially surrounded by the inflatable member 814. In some embodiments, the magnetic member 815 is coupled directly to the inflatable member 814. In some embodiments, the system 800 includes two or more magnetic members 815. In some embodiments, at least one magnetic member 815 can be disposed proximal to the inflatable member 814 and/or at least one magnetic member 815 can be disposed distal to the inflatable member 814. In some embodiments, each magnetic member 815 of a set of magnetic members 815 (e.g., two or more magnetic members 815) can be disposed within, coupled to, disposed distal of, and/or disposed proximal of the inflatable member 814.

In some embodiments, the inflatable member 814 can surround the elongated tube 812 in an inflated and/or uninflated configuration. In some embodiments, the inflatable member 814 can extend laterally from the elongated tube 812 in an inflated and/or uninflated configuration. In some embodiments, the inflatable member 814 can extend distally from the first end 811 of the elongated tube 812 in an inflated and/or uninflated configuration. In some embodiments, the inflatable member 814 can be disposed on the elongated tube 812 such that a portion of the elongated tube 812 extends distally of the inflatable member 814 when the inflatable member 814 is in an inflated and/or uninflated configuration. In some embodiments, the inflatable member 814 can have two ends (e.g., cuffs), and each end can be sealed to an outer surface of the elongated tube 812. The elongated tube 812 can define one or more inflation ports or holes such that the inflation lumen 816 can be in fluid communication with the interior of the inflatable member 814 for transitioning the inflatable member 814 between an uninflated and an inflated configuration. In some embodiments, the inflatable member 814 can be formed on or as a part of a rigid subassembly, and the rigid subassembly can receive the elongated tube 812 within an orifice of the subassembly and the elongated tube 812 can then be sealed to the subassembly.

In some embodiments, the inflatable member 814 can be formed in any suitable shape, in any suitable size, and of any suitable material. For example, the inflatable member 814 can be elliptical, spherical, cylindrical, rectangular, tear drop, or any other suitable shape. In some embodiments, the shape can be chosen based on the particular application of the system 800. For example, the shape of the inflatable member 814 may be selected to improve ultrasound visualization in particular regions of a patient’s body. Furthermore, the inflatable member 814 can be sized for improved engagement and retention between the inflatable member 814 and the guidewire assembly 820.

The inflatable member 814 can be sufficiently pliable such that the inflatable member 814 (e.g., when inflated) can be punctured (e.g., by a needle) to define a pinhole in the wall of the inflatable member 814 rather than bursting or tearing as a result of puncture. In some embodiments, the inflatable member 814 can be formed of, for example, polyurethane, silicone, and/or polyvinyl chloride (PVC). In some embodiments, the inflatable member 814 can have any suitable material properties, wall thicknesses, and/ or inflated outermost diameters.

The guidewire assembly 820 can include a guidewire 822 having a first end 821 and a second end 823 and a coupling member 824 disposed at the first end 821 of the guidewire 822. The coupling member 824 can be configured to couple to the inflatable member 814 such that, when coupled, translation of the inflation assembly 810 (e.g., translation of the elongated tube 812 via pulling on the second end 813) can translate the guidewire assembly 820. For example, if the inflatable member 814 is moved in a first direction due to a force applied to the elongated tube 812, the coupling of the coupling member 824 to the inflatable member 814 can cause the coupling member 824 and the guidewire 822 to also move in the first direction. The coupling member 824 can be configured to couple with the inflatable member 814 via, for example, being captured by the inflatable member 814, caught within an interior region of the inflatable member, or engaged with a surface of the inflatable member 814. In some embodiments, the coupling member 824 can be size to create a micropuncture in a sidewall of the inflatable member 814.

Similar to the system 100, in some embodiments, the coupling member 824 can be distinct from the guidewire 822 and fixedly coupled to the guidewire 822 (e.g., via adhesive). For example, in some embodiments, the coupling member 824 can include a first magnetic member configured to couple to a second magnetic member of the inflatable member 814. Similar to the system 100, in some embodiments, the guidewire 822 can include the coupling member 824. Similar to the system 100, in some embodiments, the coupling member 824 can be configured to transition between a first configuration for insertion and a second configuration for retention or coupling. Similar to the system 100, in some embodiments, when the coupling member 824 is within the lumen 835 of the needle 830, the needle 830 can compress the coupling member 824 such that the coupling member is in the first configuration. Similar to the system 100, in some embodiments, the coupling member 824 can be configured to be translated in a first direction by the inflatable member 814 if a translation force on the inflatable member 814 (e.g., a translating force on the inflatable member 814 and/or a force holding the inflatable member 814 stationary) is greater than a force in a direction opposite of the translation force on the coupling member 824. If the force on the coupling member 824 is opposite and greater than the translation force on the inflatable member 814, the coupling member 824 and the inflatable member 814 can be configured to decouple.

Similar to the system 100, in some embodiments, the coupling member 824 can be configured to pierce the inflatable member 814 such that the coupling member 824 can be inserted into and/or through the inflatable member 814. Similar to the system 100, the optional barrier member 895 may be a portion of the 810 inflation assembly 810 that is more resistant to puncturing or tearing (e.g., by a needle) than the inflatable member 814 or a portion of the inflatable member 814. Similar to the system 100, in some embodiments, the inflatable member 814 can be filled and/or inflated with a fluid (e.g., a liquid or a gaseous fluid) after being disposed in the upper trachea of the patient. For example, the inflatable member 814 can be filled and/or inflated with a fluid and/or contrast medium such that the inflatable member 814 defines an echogenic space detectable using ultrasound imaging.

In use, an end of a tubular member (e.g., an ETT) can be inserted through an orifice of a patient (e.g., a nose or mouth of a patient), through a cricoid ring of the patient, and to a location beyond a target region of the trachea of the patient such an inflatable member of the tubular member is disposed between the target region of the trachea and the carina of the patient. The target region of the trachea can include a desired tracheal puncture site (e.g., a location between the second and third tracheal rings of the patient or a location between the first and second tracheal rings of the patient). The inflatable member of the tubular member can be transitioned from an undeployed configuration to an expanded configuration such that the inflatable member sealingly contacts the trachea of the patient such that air cannot flow between the lungs of the patient and the orifice of the patient except through a lumen of the tubular member. The tubular member can be fluidically coupled to a ventilator such that the lungs of the patient can be ventilated by the ventilator via the tubular member. Thus, the target region of the trachea external to the tubular member is fluidically isolated from a region of the airway opposite the location of the expanded inflatable member.

The first end 811 of the elongated tube 812 of the inflation assembly 810 can then be inserted through an orifice of the patient (e.g., a nose or mouth of a patient), between the cricoid ring and an outer surface of the tubular member, and to the target region of the trachea of the patient such that the inflatable member 814 is disposed between the outer surface of the tubular member and the inner surface of the trachea. Thus, the elongated tube 812 is disposed next to the tubular member through the larynx and a portion of the trachea of the patient. In some embodiments, a user can determine that the first end 811 of the elongated tube 812 is extended a particular distance into the trachea based on the known relative lengths of the tubular member 850 and the elongated tube 812 and/or markings on at least one of the tubular member 850 or the elongated tube 812.

In some embodiments, the inflatable member 814 can be translated through the patient to the target region of the trachea such that the inflatable member 814 is disposed on an anterior side of the tubular member (e.g., between the outer surface of the tubular member and the anterior wall of the upper trachea). For example, the elongated tube 812 can be manipulated during insertion of the elongated tube 812 to maintain the inflatable member 814 on an anterior side of the tubular member. In some embodiments, the one or more optional magnet members 815 can be used to urge the inflatable member 814 to a position anterior of the tubular member. For example, the optional external magnetic assembly 840 can be placed on the anterior neck of the patient such that the one or more magnetic members 815 of the inflation assembly 810 are urged toward the external magnetic assembly 840 such that the first end 811 of the elongated tube 812 and/or the inflatable member 814 is urged into a position anterior of the tubular member (e.g., between the tubular member and the anterior wall of the upper trachea).

Fluid can then be delivered to the inflatable member 814 via the inflation lumen 816. As described above, the fluid can include a fluid and/or contrast medium such that the inflatable member 814 is detectable via imaging (e.g., ultrasound). In some embodiments, the inflatable member 814 can be sized relative to the size (e.g., diameter) of the trachea and size (e.g., diameter) of the tubular member such that inflating the inflatable member 814 from an undeployed to an expanded configuration causes a portion of the outer surface of the inflatable member 814 to contact the anterior surface of the trachea such that the contacting portion of the outer surface and all tissue planes between the inflatable member 814 and the external surface of the patient on which the ultrasound probe 860 is disposed (e.g., the ultrasound probe 860 being aligned with the inflatable member 814) can be visualized by ultrasound. In some embodiments, in the expanded configuration, the inflatable member 814 can apply a compressive force against the tissue planes between the inflatable member 814 and the external surface of the patient. In some embodiments, the optional one or more magnet members 815 can be used to urge the inflatable member 814 against the anterior surface of the upper trachea. For example, the optional external magnetic assembly 840 can be placed on the anterior neck of the patient such that the one or more magnetic members 815 of the inflation assembly 810 are urged toward the external magnetic assembly 840 such that the expanded inflatable member 814 is urged into contact with the anterior wall of the upper trachea. In some embodiments, in the expanded configuration, the inflatable member 814 can apply a compressive force against the tissue planes between the inflatable member 814 and the external surface of the patient.

The inflatable member 814 can then be visualized (e.g., using the ultrasound probe 860) such that the location of the inflatable member 814 can be identified. If necessary, the external magnetic assembly 840 can then be moved along the skin of the anterior neck of the patient to urge the inflatable member 814 toward a desired tracheal puncture site by urging one or more magnetic members 815 in the same direction. In some embodiments, the ultrasound probe 860 can be used to determine a tracheal puncture site between particular tracheal rings (e.g., between the first and second tracheal rings of the patient or between the second and the third tracheal rings of the patient). In some embodiments, alternatively or in addition to urging the inflatable member 814 to a desired tracheal puncture site using the external magnetic assembly 840, the inflatable member 814 can be urged to a desired tracheal puncture site by advancing or retracting the elongated tube 812 relative to the patient (e.g., by pulling or pushing a portion of the elongated tube 812 outside of the patient’s body near the patient’s head).

While visualizing the location of the inflatable member 814 using the ultrasound probe 860, the guidewire assembly 820 can be inserted through the anterior neck of the patient and into the patient’s trachea and coupled to the inflatable member 814. For example, the needle 830 can be inserted through the anterior neck and trachea of the patient and through a sidewall of the inflatable member 814 such that the first end 831 of the needle 830 (e.g., the tip) is disposed within the inflatable member 814. During insertion of the needle 830, the ultrasound probe 860 can be used to visualize the needle 830 and any intervening patient structure between the skin of the patient and the inflatable member 814. For example, the ultrasound probe 860 can be used to identify the thyroid isthmus and proximal blood vessels of the patient in real-time during insertion of the needle 830 such that the thyroid isthmus and proximal blood vessels can be avoided. Furthermore, the ultrasound probe 860 can be used to confirm that the first end 831 of the needle 830 is disposed within the inflatable member 814. Additionally or alternatively, echogenic fluid can be aspirated from the inflatable member 814 via the needle 830 (e.g., into a syringe barrel) to verify that the first end 831 of the needle 830 is disposed within the inflatable member 814.

With the first end 831 of the needle 830 disposed within the inflatable member 814, the coupling member 824 and a portion of the guidewire 822 can be inserted through the lumen 835 of the needle 830 and translated (e.g., pushed) through the lumen 835. The coupling member 824 can then be translated out from the first end 831 of the needle 830 such that the coupling member 824 is disposed within the inflatable member 814. The needle 830 can then be withdrawn from the patient via translation of the needle 830 relative to the coupling member 824 and the guidewire 822, leaving the coupling member 824 within the inflatable member 814 and the guidewire 822 extending through a wall of the inflatable member 814. Additionally, in embodiments in which the external magnetic assembly 840 is used, the external magnetic assembly 840 can be removed from the patient such that the one or more magnetic members 815 are no longer urged (e.g., via magnetic attraction) toward the anterior tracheal wall. Additionally, the inflatable member 814 can be deflated.

With the guidewire assembly 820 extending through the anterior neck of the patient and coupled to the inflatable member 814, any suitable percutaneous tracheostomy procedure can be performed using the guidewire assembly 820 and the tract through the patient’s anterior neck to the patient’s trachea through which the guidewire assembly 820 is disposed. For example, the elongated tube 812 can be translated such that the first end 811 of the elongated tube 812 moves out of an orifice (e.g., nasal or oral) of the patient. Thus, the coupling member 824 of the guidewire assembly 820 is translated through the cricoid ring and out of the orifice of the patient such that the guidewire 822 extends through the tracheal puncture site, through the larynx, and out of the patient’s nasal or oral orifice. A puncture dilator can be translated over the guidewire 822 to dilate the anterior tracheostomy tract within which the guidewire 822 is disposed. A first end of a second guidewire can be translated through the puncture dilator. The second guidewire can, for example, have a greater diameter than the guidewire 822. For example, in some embodiments, the second guidewire can be a 0.052” percutaneous tracheostomy guidewire. With the first end of the second guidewire disposed between the anterior surface of the trachea and the outer surface of the tubular member, the puncture dilator can be removed. The ventilator coupled to the tubular member can be stopped, the inflatable member coupled to the tubular member can be deflated, and the tubular member can be removed from the patient via the orifice through which the tubular member was inserted. While the patient is apnic (and viral aerosolization is minimized) due to being disconnected from a functioning ventilator (e.g., due to the ventilator being stopped, the tubular member being removed, and a tracheostomy tube coupled to a ventilator not yet being operably coupled to the tracheal puncture site), the guidewire 822 can remain extending through the tracheal puncture site and out of the mouth or nose of the patient. Thus, if reintubation is necessary, a tubular member (e.g., an ETT) can be threaded over the guidewire 822 to quickly place the patient back on a ventilator. After the tubular member is removed, a tracheal tract dilator can be advanced over the second guidewire to externally dilate the tract through the anterior neck. After the dilator is removed, a tracheostomy tube can be advanced over the guidewire and into engagement with the tract through the patient’s tracheal wall such that a first end of the tracheostomy tube is disposed with in the upper trachea and a second end is disposed outside of the patient (e.g., extending from the anterior neck of the patient). The second guidewire can be removed. A balloon cuff of the tracheostomy tube disposed within the trachea can be inflated to seal the trachea around the tracheostomy tube. A ventilator can be operably coupled to the tracheostomy tube such that air is provided to the patient’s lungs via the tracheostomy tube and the apnic period is ended. The guidewire 822 can be removed from the patient (e.g., via cutting off the coupling member 824 and pulling an end of the guidewire 822 such that it is removed from either the tracheal puncture site or the orifice (e.g., nose or mouth).

In some embodiments, for example, the inflatable member 814 can be elliptical in shape and formed of a low durometer urethane. The inflatable member 814 can have an outermost diameter ranging from about 15 mm to about 55 mm in an inflated configuration, and a length of about 55 mm. The wall thickness at the maximum balloon diameter in the inflated configuration can be between about 0.029 mm and about 0.038 mm. The inflatable member 114 can be filled with up to, for example, about 20 ml to about 40 ml of fluid in the inflated configuration.

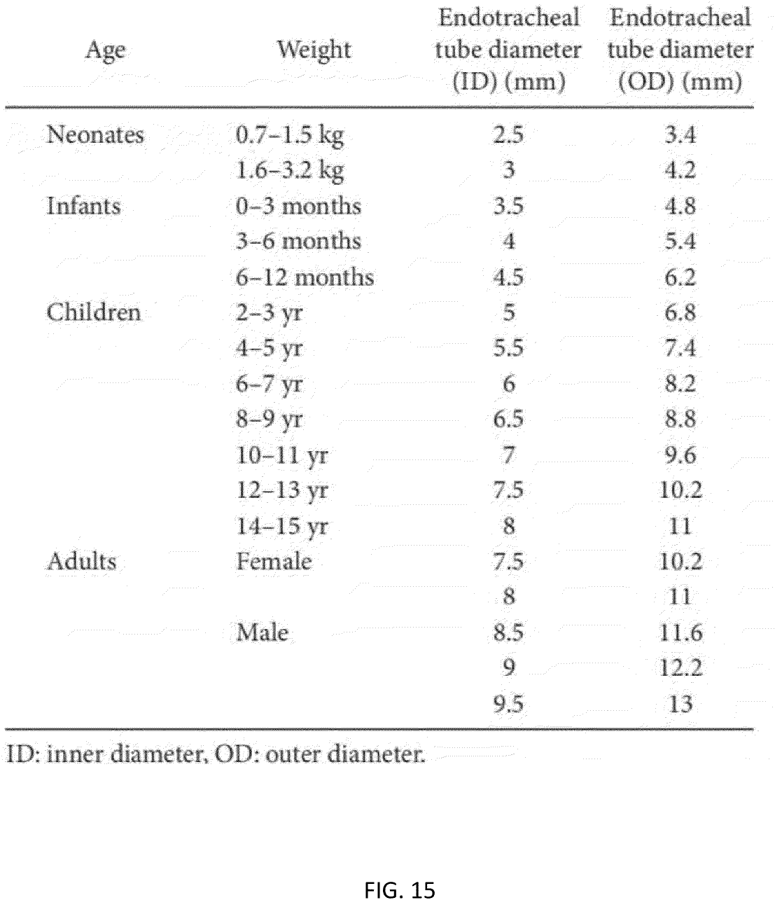

In some embodiments, the outer diameter of the inflatable member 814 can depend on the outer diameter of the tubular member (e.g., the ETT) and the inner diameter of the trachea of the patient. For example, as shown in FIG. 15, patients of different ages and stages of development can tolerate different ETT sizes. Typical tracheal diameters range, for example, between about 15 mm and about 20 mm. Generally, older and/or larger patients can receive an endotracheal tube having a larger outer diameter than younger and/or smaller patients. Thus, the outer diameter of the inflatable member 814 in the expanded configuration can be selected depending on the outer diameter of the ETT disposed within the patient’s trachea and the inner diameter of the trachea such that the inflatable member 814 can be either movable relative to the ETT to press against the anterior tracheal wall using the external magnetic assembly 850 or such that the inflatable member 814 can be expanded to be sufficiently large to fill the space between the ETT and the anterior tracheal wall and apply pressure to the anterior tracheal wall due to the expansion of the inflatable member 814 away from the anterior tracheal wall being constrained by the ETT.

In some embodiments, the inflatable member 814 can be collapsed sufficiently small in the undeployed configuration such that the inflatable member 814 can be translated through a lumen of a tubular member (e.g., an ETT) for inline delivery (e.g., similarly as described with respect to the system 300 shown in FIGS. 3A-3M above), and the inflatable member 814 can be expandable to a sufficiently large size in the expanded configuration such that the same inflatable member 814 can be used for paired delivery such that the inflatable member 814 can be expanded in a space between the tubular member and the anterior tracheal wall and apply pressure against the anterior tracheal wall for coaptation and to define an echogenic space that can be visualized via ultrasound. Thus, in some embodiments, the same inflation assembly 810 can be used for both an inline delivery procedure and a paired delivery procedure (e.g., a clinician can determine which procedure is more appropriate for the patient based on the patient’s anatomy and risk factors). For example, the size (e.g., largest lateral extent or diameter) of the inflatable member 814 in the undeployed configuration can be small enough to be passed through a lumen of an ETT having an inner diameter as shown in the chart in FIG. 15 and the inflatable member 814 can be large enough in the deployed configuration to apply coaptive pressure against the anterior tracheal wall of the patient due to being constrained between the anterior tracheal wall and the ETT in the deployed configuration, the ETT having an outer diameter corresponding to the inner diameter as shown in the chart of FIG. 15. In some embodiments, the inflatable member 814 to be used can have a size in the undeployed and expanded configurations that depends on the inner diameter and outer diameter of the associated ETT (examples shown in FIG. 15) and the inner diameter of the patient’s trachea. In some embodiments, a Foley catheter may be adequate to be used as the inflation assembly 810 for delivery to a space in the upper trachea A outside of a tubular member (e.g., an ETT), for coaptation between the balloon of the Foley catheter and the anterior tracheal wall, and for ultrasound guidance. For example, a Foley catheter may be used as the inflation assembly 810 for patients with anatomical dimensions suited to the shape and size of a balloon of the Foley catheter and/or as part of a system having components (e.g., the needle 830 and/or the guidewire 822) configured to functionally operate as described herein in combination with a Foley catheter.

In some embodiments, the inflatable member 814 can be formed as a double layered balloon, such as the balloons disclosed in International Patent Application No. PCT/US17/026141, incorporated by reference above. For example, the inflatable member 814 can include an inner balloon and an outer balloon, the inner balloon disposed inside the outer balloon. In some embodiments, the inflatable member 814 can include a set of struts disposed between the inner balloon and the outer balloon to maintain a distance or space between the inner balloon and the outer balloon. The inner balloon can be fluidically coupled to a first inflation lumen (e.g., inflation lumen 816) and the outer balloon can be fluidically coupled to a second inflation lumen. Both the first inflation lumen and the second inflation lumen can be included within and/or defined by the elongated tube 812. To transition the double layered inflatable member 814 from the undeployed configuration to the expanded configuration, the inner balloon can be inflated with gaseous fluid (e.g., air) and the outer balloon can be inflated with liquid fluid and/or contrast medium such that the liquid fluid fills the space between the inner surface of the outer balloon and the outer surface of the inner balloon. The liquid fluid filled space between the inner balloon and the outer balloon can be used as an echogenic window, with the pressure of the gaseous fluid in the inner balloon and the pressure of the liquid fluid in the outer balloon both pushing the outer surface of the outer balloon against the anterior tracheal wall to create coaptation.

Since the double layered inflatable member 814 can define the echogenic window using less liquid fluid than a single layered inflatable member due to the inner balloon being filled with gaseous fluid, the risk of a volume of fluid sufficient to harm a patient being released from the inflatable member 814 when the needle 830 is passed through the outer balloon and/or the inner balloon is reduced compared to a single layer balloon filled with liquid fluid. In some embodiments, the double layered inflatable member 814 can include one or more spacers disposed between the inner balloon and the outer balloon to maintain a preferred distance between the inner balloon and the outer balloon (e.g., to prevent the outer balloon from contacting the inner balloon and thus eliminating the echogenic window). For example, the one or more spacers can include rivets located around the surface of the inner balloon. In some embodiments, the rivets can be disposed to maintain a distance between the inner balloon and the outer balloon in all directions. In some embodiments, the rivets can be disposed to maintain a distance between the inner balloon and the outer balloon in an area of the inflatable member 814 in which an echogenic window is desired (e.g., a central portion).

In some embodiments, the double layered inflatable member 814 can be sufficiently large such that no magnets are needed to draw the double layered inflatable member 814 against the anterior tracheal wall to create coaptation and define an ultrasound visible echogenic window. Instead, the double layered inflatable member 814 can have a sufficiently large perimeter (e.g., be sufficiently large in diameter or cross-sectional area) in the expanded configuration such that the inflatable member 814 fills a distance or gap between the tubular member (e.g., the ETT) and the anterior tracheal wall and presses against both the tubular member and the anterior tracheal wall sufficiently to create coaptation against the anterior tracheal wall and defines an echogenic window. For example, the outer diameter of the tubular member in an adult patient may range from 10-12 mm, and the inflatable member 814 can be sized to have an outer diameter in an unrestrained expanded configuration that is larger than the inner diameter of the patient’s trachea minus the outer diameter of the tubular member. In some embodiments, the system 800 can include a magnet associated with the inflatable member 814 or the elongated tube 812 that is only used to position the double layered inflatable member 814 on an anterior side of the tubular member prior to transitioning the double layered inflatable member 814 from the undeployed to the expanded configuration.

In some embodiments, the inflatable member 814 can define passageways (not shown) (also referred to as “vents”) extending from a first end to a second end of the inflatable member 814 such that fluid (e.g., air) can travel through the passageways when the inflatable member 814 is in the expanded configuration. For example, in some embodiments, when the inflatable member 814 is disposed in the trachea in the expanded configuration, each passageway can be partially bounded by the inflatable member 814 and partially bounded by the patient’s tracheal wall. Thus, air can flow between a portion of the trachea on a first side of the inflatable member 814 (e.g., a region between the cricoid ring and the inflatable member 814) and a portion of the trachea on a second side of the inflatable member 814 (e.g., a region between the inflatable member 814 and the lungs of the patient) through the passageways when the inflatable member 814 is in the expanded configuration. In some embodiments, the inflatable member 814 can define the entire outer boundary of one or more of the passageways. The inflatable member 814 can be shaped to define any suitable number of passageways in the expanded configuration (e.g., one, two three, four, or more passageways). The passageways can be defined in any suitable location of the inflatable member 814. In some embodiments, the inflatable member 814 can define a suitable number of passageways having a suitable size (e.g., having a suitable combined cross-sectional area) such that a patient can be properly ventilated (e.g., freely or via a ventilator) through the passageways when the inflatable member 814 is disposed in a trachea of the patient and the outer surface of the inflatable member 814 contacts the inner wall of the trachea of the patient. For example, the outer surface of the inflatable member 814 can contact the inner wall of the trachea of the patient in an uninterrupted manner such that air cannot flow between the outermost surface of the inflatable member 814 and the tracheal wall and/or the tubular member 850 or the rate that air can flow between the outermost surface and the tracheal wall and/or the tubular member 850 is insufficient for proper ventilation and oxygenation. In some embodiments, the combined cross-sectional area of the passageways can be sufficiently large to allow for between about 40 and about 75 liters per minute of airflow through the passageways. Thus, with the inflatable member 814 deployed in the trachea, the patient can receive closed and continuous ventilation via the passageways, including during ultrasound visualization (e.g., via Coaptive Ultrasound) of the inflatable member 814 and/or tissue between the inflatable member 814 and an ultrasound probe.

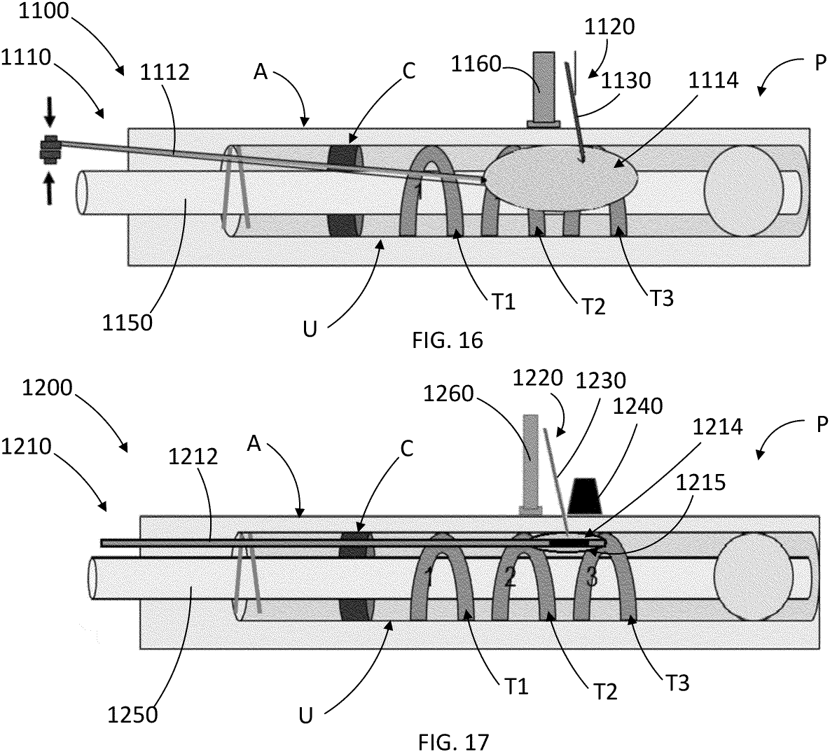

In some embodiments, the inflatable member 814 can include a single layered balloon that is sufficiently large in the expanded configuration that the inflatable member 814 presses against the anterior tracheal wall and the tubular member (e.g., the ETT) when the inflatable member 814 is transitioned from the undeployed to the expanded configuration. Thus, in some embodiments, the system 800 can include no magnet associated with the inflatable member 814 or the elongated tube 812, as the inflatable member 814 fills the gap between the tubular member and the anterior tracheal wall as the inflatable member 814 expands and forms an echogenic window due to being pressed against the anterior tracheal wall. Alternatively, in some embodiments, the system 800 can include a magnet associated with the inflatable member 814 or the elongated tube 812 that is only used to position the inflatable member 814 on an anterior side of the tubular member prior to transitioning the inflatable member 814 from the undeployed to the expanded configuration. For example, FIG. 16 is a schematic illustration of a system 1100. The system 1100 can be the same or similar in structure and/or function to any of the systems described herein, such as the system 800. For example, the system 1100 includes an inflation assembly 1110, a guidewire assembly 1120, and a needle 1130. The system 1100 can optionally also include an ultrasound probe 1160. The inflation assembly 1110 can be the same or similar in structure and/or function to any of the inflation assemblies described herein. The inflation assembly 1110 can include an elongated tube 1112, and an inflatable member 1114. As shown in FIG. 16, the inflatable member 1114 can include a single layered balloon that is sufficiently large in the expanded configuration that the inflatable member 1114 presses against the anterior tracheal wall and the tubular member 1150 (e.g., the ETT) when the inflatable member 1114 is transitioned from the undeployed to the expanded configuration. Thus, the inflatable member 1114 fills the gap between the tubular member 1150 and the anterior tracheal wall as the inflatable member 1114 expands and forms an echogenic window due to being pressed against the anterior tracheal wall. As shown in FIG. 17, the inflatable member 1114 can then be pierced by the needle 1130 and the guidewire assembly 1120 can be coupled to the inflatable member 1114 via the needle 1130.

In some embodiments, the inflatable member 814 can include a single layered balloon and the inflatable member 814 and/or the elongated tube 812 can include the one or more magnetic members 815 such that the inflatable member 814 can be drawn toward or against the anterior tracheal wall by the external magnetic assembly. The single layered balloon can be sufficiently small such that it is movable within the patient’s trachea relative to the tubular member (e.g., the ETT) in the deployed configuration. For example, FIG. 17 is a schematic illustration of a system 1200. The system 1200 can be the same or similar in structure and/or function to any of the systems described herein, such as the system 800. For example, the system 1200 includes an inflation assembly 1210, a guidewire assembly 1220, and a needle 1230. The system 1200 can optionally also include an external magnetic assembly 1240 and an ultrasound probe 1260. The inflation assembly 1210 can be the same or similar in structure and/or function to any of the inflation assemblies described herein. The inflation assembly 1210 can include an elongated tube 1212, an inflatable member 1214, and a magnetic member 1215. As shown in FIG. 17, the inflatable member can include a single layered balloon and the inflatable member 1214 and/or the elongated tube 1212 can include the one or more magnetic members 1215 such that the inflatable member 1214 can be drawn toward or against the anterior tracheal wall by the external magnetic assembly 1240. The single layered balloon can be sufficiently small such that it is movable within the patient’s trachea relative to the tubular member 1250 (e.g., the ETT) in the deployed configuration. As shown in FIG. 17, the inflatable member 1214 can then be pierced by the needle 1230 and the guidewire assembly 1220 can be coupled to the inflatable member 1214 via the needle 1230.

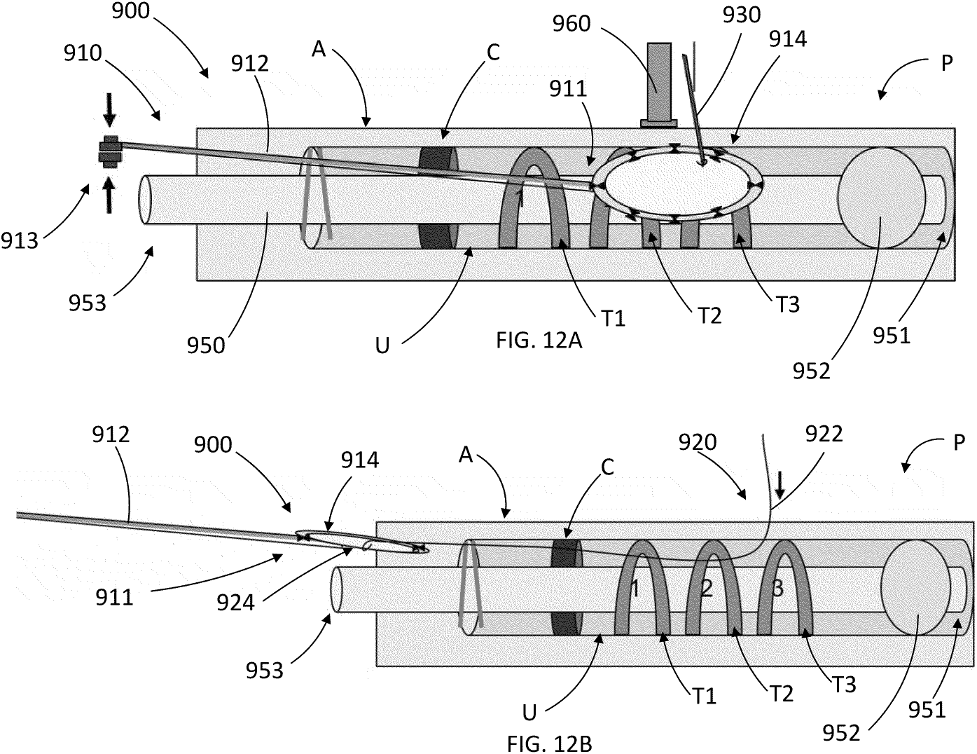

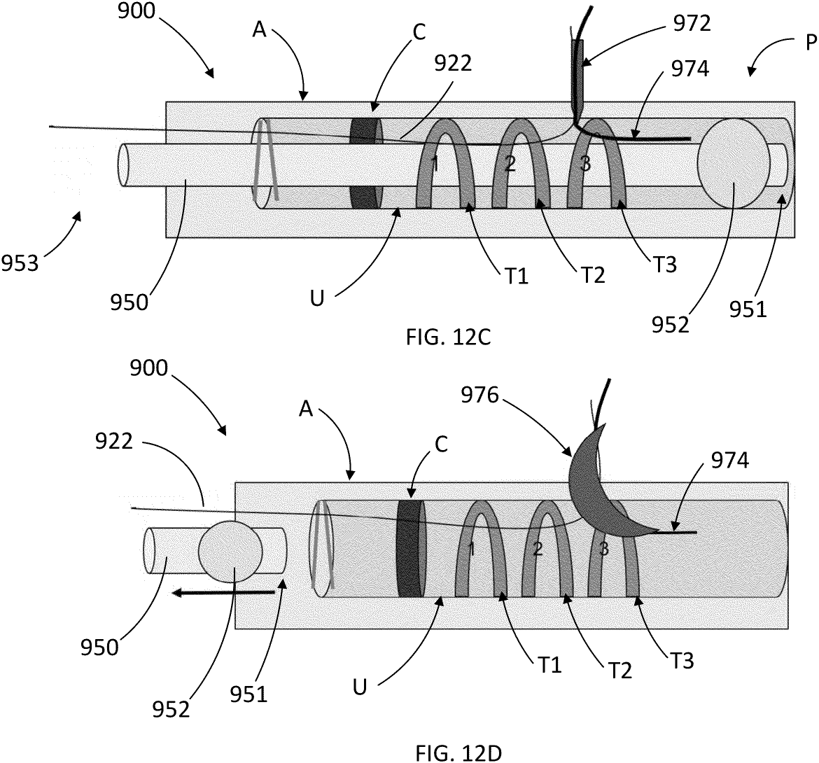

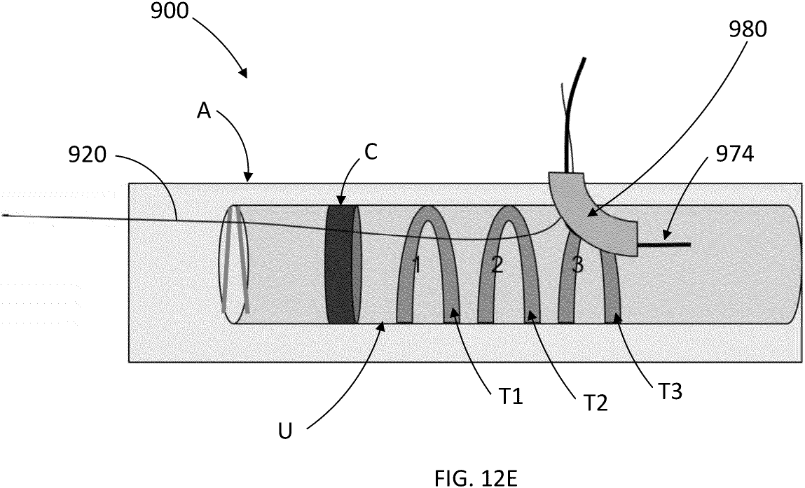

FIGS. 12A-12E are schematic illustrations of a guidewire placement system 900 in various stages of operation. The guidewire placement system 900 can be the same or similar in structure and/or function to the guidewire placement system 800 described above. For example, the system 900 includes an inflation assembly 910 and a guidewire assembly 920. The system 900 also includes an ultrasound probe 960. The inflation assembly 910 can include an elongated tube 912 and an inflatable member 914. The elongated tube 912 can have a first end 911 and a second end 913. In some embodiments, the elongated tube 912 can have a length sufficient to extend from at least an oral or nasal orifice of a patient to the trachea of the patient. The inflatable member 914 can be coupled to the elongated tube 912 at or near the first end 911 of the elongated tube 912. The inflation member 914 can be a double layered balloon inflation member with struts as described above. The inflation assembly 910 can include a first inflation lumen in fluid communication with an inner balloon of the inflatable member 914 and a second inflation lumen in fluid communication with the outer balloon of the inflation member 914. In some embodiments, the inflation lumen 916 can be disposed within and/or be defined by the elongated tube 912.

The first end 951 of a tubular member 950 (e.g., an ETT) can be inserted through an orifice (not shown) of a patient P (e.g., a nose or mouth of a patient), through a cricoid ring C of the patient P, and into the upper trachea U of the patient P such that the first end 951 and the inflatable member 952 of the tubular member 950 are disposed between a third tracheal ring T3 and a carina of a patient. For example, the first end 951 of the tubular member 950 can be advanced through the patient under direct or indirect laryngoscopy. The inflatable member 952 can be transitioned to the inflated configuration such that the inflatable member 952 can secure the first end 951 within the trachea and/or such that the first end 951 of the tubular member 950 can be stabilized (e.g., resistant to axial movement) relative to the inner surface of the tracheal wall. The second end 953 of the tubular member 950, which is disposed outside of the patient P, can be operably coupled to a mechanical ventilator such that the lungs of the patient can be ventilated by the ventilator via the tubular member 950. In some embodiments, the tubular member 950 is disposed as shown in FIG. 12A for a period of time (e.g., hours, days) prior to delivering the elongated tube 912 to a desired tracheal puncture site in a target region of the trachea. The target region of the trachea external to the tubular member 950 is fluidically isolated from a region of the airway opposite the location of the expanded inflatable member 952 (e.g., the lungs).

The first end 911 of the elongated tube 912 of the inflation assembly 910 can then be inserted through an orifice of the patient P (e.g., a nose or mouth of a patient P), between the cricoid ring and an outer surface of the tubular member 950, and to the target region of the trachea of the patient P such that the inflatable member 914 is disposed between the outer surface of the tubular member 950 and the inner surface of the trachea. Thus, as shown in FIG. 12A, the elongated tube 912 is disposed next to the tubular member 950 through the larynx and a portion of the trachea of the patient P.

Gaseous fluid can then be delivered to the inner balloon of the inflatable member 914 via the first inflation lumen and liquid fluid can be delivered to the outer balloon of the inflatable balloon 914 via the second inflation lumen. As shown in FIG. 12A, the inflatable member 914 can be sized relative to the size (e.g., diameter) of the trachea and size (e.g., diameter) of the tubular member such that inflating the inflatable member 914 from an undeployed to an expanded configuration causes a portion of the outer surface of the outer balloon of the inflatable member 914 to contact the surface of the anterior wall of the trachea such that the contacting portion of the outer surface and all tissue planes between the inflatable member 914 and the external surface of the patient on which the ultrasound probe 960 is disposed (e.g., the ultrasound probe 960 being aligned with the inflatable member 914) can be visualized by ultrasound. In some embodiments, in the expanded configuration, the inflatable member 914 can apply a compressive force against the tissue planes between the inflatable member 914 and the external surface of the patient P. In some embodiments, in the expanded configuration, the inflatable member 914 can apply a compressive force against the tissue planes between the inflatable member 914 and the external surface of the patient P. The inflatable member 914 can then be visualized (e.g., using the ultrasound probe 960) such that the location of the inflatable member 914 can be identified. In some embodiments, the ultrasound probe 960 can be used to determine a tracheal puncture site between particular tracheal rings (e.g., between the first tracheal ring T1 and second tracheal ring T2 of the patient P or between the second tracheal ring T2 and the third tracheal ring T3 of the patient P). In some embodiments, the inflatable member 914 can be urged to a desired tracheal puncture site by advancing or retracting the elongated tube 912 relative to the patient (e.g., by pulling or pushing a portion of the elongated tube 912 outside of the patient’s body near the patient’s head).

As shown in FIG. 12A, while visualizing the location of the inflatable member 914 using the ultrasound probe 960, a needle 930 can be inserted through the anterior neck A of the patient P and into the sidewall of the inflatable member 914 such that the first end of the needle 930 (e.g., the tip) is disposed within the inflatable member 914. During insertion of the needle 930, the ultrasound probe 960 can be used to visualize the needle 930 and any intervening patient structure between the skin of the patient and the inflatable member 914. For example, the ultrasound probe 960 can be used to identify the thyroid isthmus and proximal blood vessels of the patient in real-time during insertion of the needle 930 such that the thyroid isthmus and proximal blood vessels can be avoided. Furthermore, the ultrasound probe 960 can be used to confirm that the first end of the needle 930 is disposed within the inflatable member 914. Additionally or alternatively, echogenic fluid can be aspirated from the inflatable member 914 via the needle 930 (e.g., into a syringe barrel) to verify that the first end of the needle 930 is disposed within the inflatable member 914.

With the first end of the needle 930 disposed within the inflatable member 914, a coupling member 924 and a portion of a guidewire 922 of the guidewire assembly 920 can be inserted into a lumen of the needle 930 and translated (e.g., pushed) through the lumen. The coupling member 924 can then be translated out from the first end of the needle 930 such that the coupling member 924 is disposed within the inflatable member 914. The needle 930 can then be withdrawn from the patient P via translation of the needle 930 relative to the coupling member 924 and the guidewire 922, leaving the coupling member 924 within the inflatable member 914 and the guidewire 922 extending through a wall of the inflatable member 914. Additionally, the inflatable member 914 can be deflated by drawing the gaseous and liquid fluid from the inflatable member 914.

As shown in FIG. 12B, with the guidewire assembly 920 extending through the anterior neck A of the patient P and coupled to the inflatable member 914, the elongated tube 912 can be translated such that the first end 911 of the elongated tube 912 moves out of an orifice (e.g., nasal or oral) of the patient P. Thus, the coupling member 924 of the guidewire assembly 920 is translated through the cricoid ring C and out of the orifice of the patient P such that the guidewire 922 extends through the tracheal puncture site, through the larynx, and out of the patient’s nasal or oral orifice. In some embodiments, the guidewire 922 may be pushed while pulling on the elongated tube 912 such that the inflatable member 914 is translated without decoupling from the coupling member 924.

As shown in FIG. 12C, a puncture dilator 972 can be translated over the guidewire 922 to dilate the anterior tracheostomy tract within which the guidewire 922 is disposed. If a larger guidewire is desired for tract dilatation, a first end of a second guidewire 974 can be translated through the puncture dilator 972. The second guidewire 974 can, for example, have a greater diameter than the guidewire 922. For example, in some embodiments, the second guidewire 974 can be a 0.052” percutaneous tracheostomy guidewire. With the first end of the second guidewire 974 disposed between the anterior surface A of the trachea and the outer surface of the tubular member 950, the puncture dilator 972 can be removed. In some embodiments, the puncture dilator 972 can define a first lumen configured to receive the guidewire 922 such that the puncture dilator 972 can be threaded over the guidewire 922. The puncture dilator 972 can define a second lumen configured to receive the second guidewire 974. In some embodiments, the second lumen can have a larger diameter than the first lumen.

As shown in FIG. 12D, the ventilator coupled to the tubular member 950 can be stopped, the inflatable member 952 coupled to the tubular member 950 can be deflated, and the tubular member 950 can be removed from the patient P via the orifice through which the tubular member 950 was inserted. While the patient is apnic (and viral aerosolization is minimized) due to being disconnected from a functioning ventilator (e.g., due to the ventilator being stopped, the tubular member 950 being removed, and a tracheostomy tube coupled to a ventilator not yet being operably coupled to the tracheal puncture site), the guidewire 922 can remain extending through the tracheal puncture site and out of the mouth or nose of the patient. Thus, if reintubation is necessary, a tubular member (e.g., an ETT) can be threaded over the guidewire 922 to quickly place the patient back on a ventilator. After the tubular member 950 is removed, a tracheal tract dilator 976 can be advanced over the second guidewire 974 to externally dilate the tract through the anterior neck A.

As shown in FIG. 12E, after the dilator 976 is removed, a tracheostomy tube 980 can be advanced over the second guidewire 974 and into engagement with the tract through the patient’s tracheal wall such that a first end of the tracheostomy tube 980 is disposed with in the upper trachea U and a second end is disposed outside of the patient P (e.g., extending from the anterior neck A of the patient P). The second guidewire 974 can be removed. A ventilator can be operably coupled to the tracheostomy tube 980 such that air is provided to the patient’s lungs via the tracheostomy tube 980 and the apnic period is ended. The guidewire 922 can be removed from the patient (e.g., via cutting off the coupling member 924 and pulling an end of the guidewire 922 such that it is removed from either the tracheal puncture site or the orifice (e.g., nose or mouth).

Although not shown, in some embodiments, rather than threading the second guidewire 974 through the puncture dilator 972 and then advancing the tracheal tract dilator 976 over the second guidewire 974, the tracheal tract dilator 976 can be advanced over the first guidewire 922 after removal for the puncture dilator 972. Additionally, in some embodiments, the tracheostomy tube 980 can be advanced over the first guidewire 922. Thus, only one guidewire may be needed for dilation of the tract through the anterior neck A and engagement of the tracheostomy tube 980 with the tract.

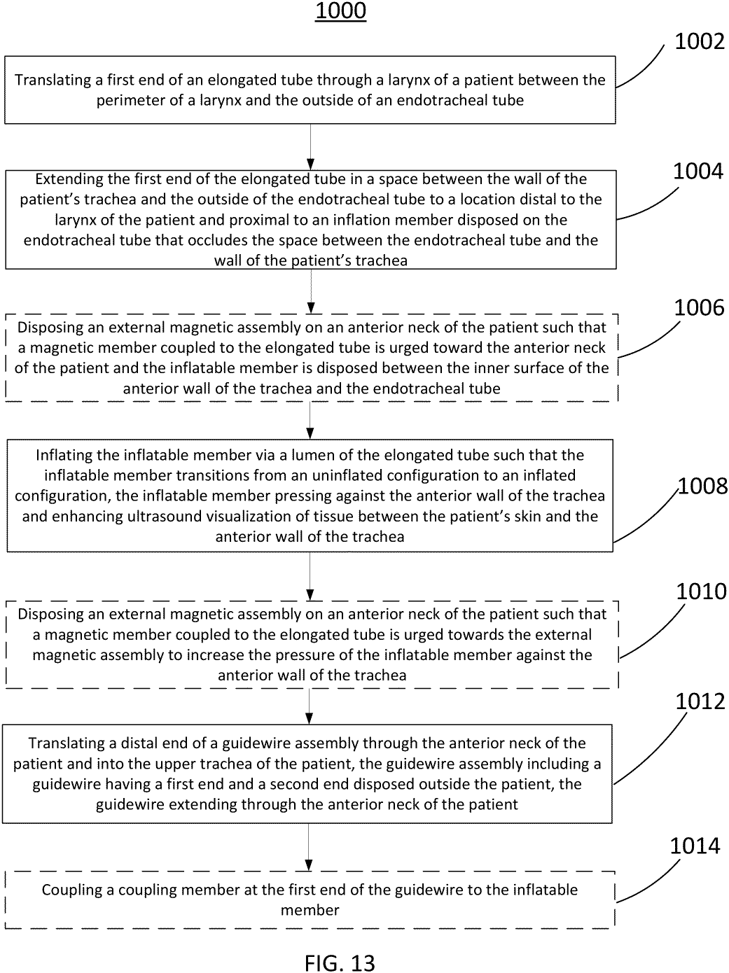

FIG. 13 is a flow chart of a method 1000, according to an embodiment. The method 1000 can be implemented using any of the systems or devices described herein, such as the system 800 or the system 900 described above. The method 1000 includes translating 1002 a first end of an elongated tube through a larynx of a patient between a perimeter of the larynx and the outside of an endotracheal tube. The first end of the elongated tube can be extended 1004 in a space between the wall of the patient’s trachea and the outside of the endotracheal tube to a location distal to the larynx of the patient and proximal to an inflation member disposed on the endotracheal tube that occludes the space between the endotracheal tube and the wall of the patient’s trachea. Optionally, an external magnetic assembly can be disposed 1006 on an anterior neck of the patient such that a magnetic member coupled to the elongated tube is urged toward the anterior neck of the patient and the inflatable member is disposed between the inner surface of the anterior wall of the trachea and the endotracheal tube. The inflatable member can then be inflated 1008 via a lumen of the elongated tube such that the inflatable member transitions from an uninflated configuration to an inflated configuration and the inflatable member presses against the anterior wall of the trachea and ultrasound visualization of tissue between the patient’s skin and the anterior wall of the trachea is enhanced. Optionally, an external magnetic assembly (such as the external magnetic assembly described in optional step 1006) can be disposed 1010 on an anterior neck of the patient such that a magnetic member coupled to the elongated tube is urged toward the external magnetic assembly to increase the pressure of the inflatable member against the anterior wall of the trachea. A distal end of a guidewire assembly can be translated 1012 through the anterior neck of the patient and into the upper trachea of the patient, the guidewire assembly including a guidewire having a first end and a second end disposed outside the patient and the guidewire extending through the anterior neck of the patient. A coupling member at the first end of the guidewire can be coupled 1014 to the inflatable member.

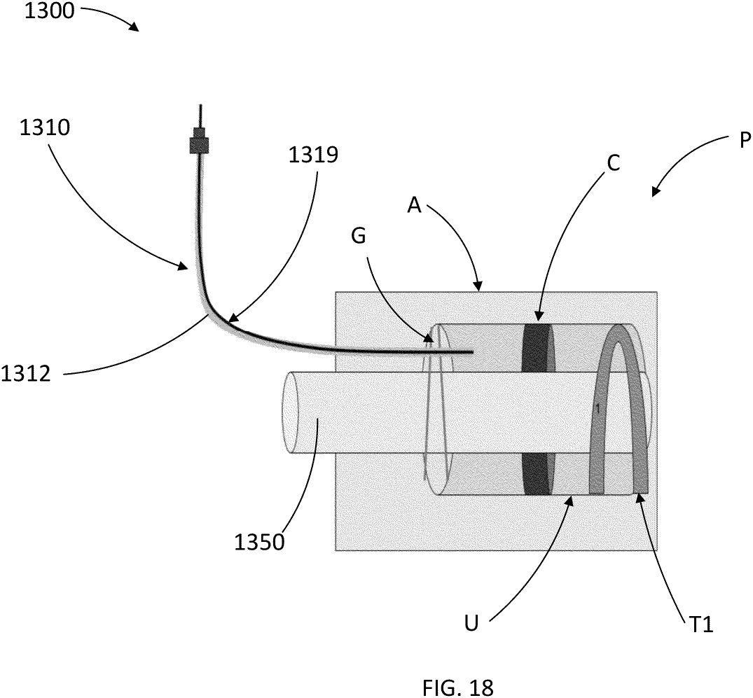

In some embodiments, a system, such as any of the systems described herein, can include a rigid or semi-rigid stylet configured to be removably received in a lumen of the elongated tube of the inflation assembly. For example, FIG. 18 is a schematic illustration of a system 1300. The system 1300 can be the same or similar in structure and/or function to any of the systems described herein, such as the system 800. For example, the system 1300 includes a tubular member 1350 and an inflation assembly 1310 including an elongated tube 1312. Although not shown, an inflatable member such as any of the inflatable members described herein can be coupled to an end of the elongated tube 1312. As shown in FIG. 18, the inflation assembly 1310 can include a stylet 1319. The stylet 1319 can be removably received within a lumen defined in the elongated tube 1312. The stylet 1319 can be shaped to assist a practitioner, while performing a direct or indirect laryngoscopy, in navigating the anatomy of the patient P to place the inflatable member of the inflation assembly 1310 in an intended portion of the upper trachea U (e.g., between the first tracheal ring T1 and the third tracheal ring (not shown)). With the stylet 1319 disposed within the elongated tube 1312, the stylet 1319 can cause the elongated tube 1312 to conform to the shape of the stylet 1319. For example, the stylet 1319 can include a curved portion disposed between two substantially straight portions (e.g., a hockey stick shape), and, with the stylet disposed in a lumen of the elongated tube 1312, the end of the elongated tube 1312 (and the associated inflatable member) can be inserted through the glottis G of the patient P (i.e., the opening between the vocal cords of the patient), even if the glottis G is small and/or disposed in a very anterior portion of the patient’s airway. Since the path between the mouth of the patient and the glottis G may include a sharp angle, the stylet can be formed to include a similar angle between the two substantially straight portions such that the glottis can be targeted. For example, the angle can be between about 80 degrees and 110 degrees. In some embodiments, the stylet 1319 may be malleable such that the angle and curvature of the stylet 1319 can be adjusted for the particular patient’s size and anatomy.

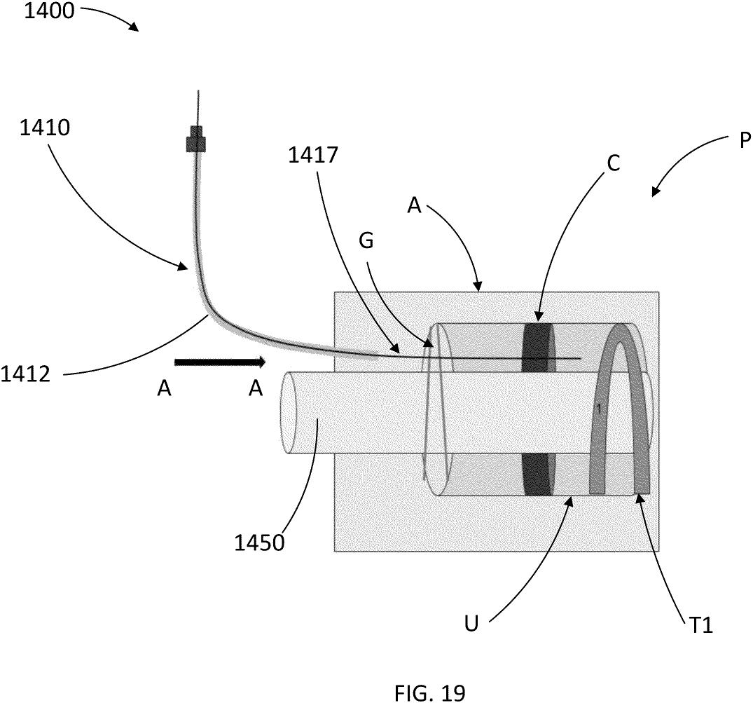

In some embodiments, such as when the glottic area is small, a guidewire can be placed through the glottis prior to introduction of an inflation assembly. For example, FIG. 19 is a schematic illustration of a system 1400. The system 1400 can be the same or similar in structure and/or function to any of the systems described herein, such as the system 800. For example, the system 1400 includes a tubular member 1450 and an inflation assembly 1410 including an elongated tube 1412. Although not shown, an inflatable member such as any of the inflatable members described herein can be coupled to an end of the elongated tube 1412. As shown in FIG. 19, the inflation assembly 1410 can include a guidewire 1317. The guidewire 1317 can be removably received within a lumen defined in the elongated tube 1412. For example, while performing a direct or indirect laryngoscopy, the guidewire 1317 can be threaded through the glottis G of the patient P and advanced until the end of the guidewire is disposed in the upper trachea U of the patient. The tubular member 1412 can then be threaded over the guidewire 1417 (e.g., in the direction of the arrow A-A) and through the glottis G to place the inflatable member of the inflation assembly 1410 in an intended portion of the upper trachea U (e.g., between the first tracheal ring T1 and the third tracheal ring (not shown)). The guidewire 1417 can then be removed from the lumen of the tubular member 1412 be pulling the guidewire 1417 out of the proximal end of the tubular member 1412.

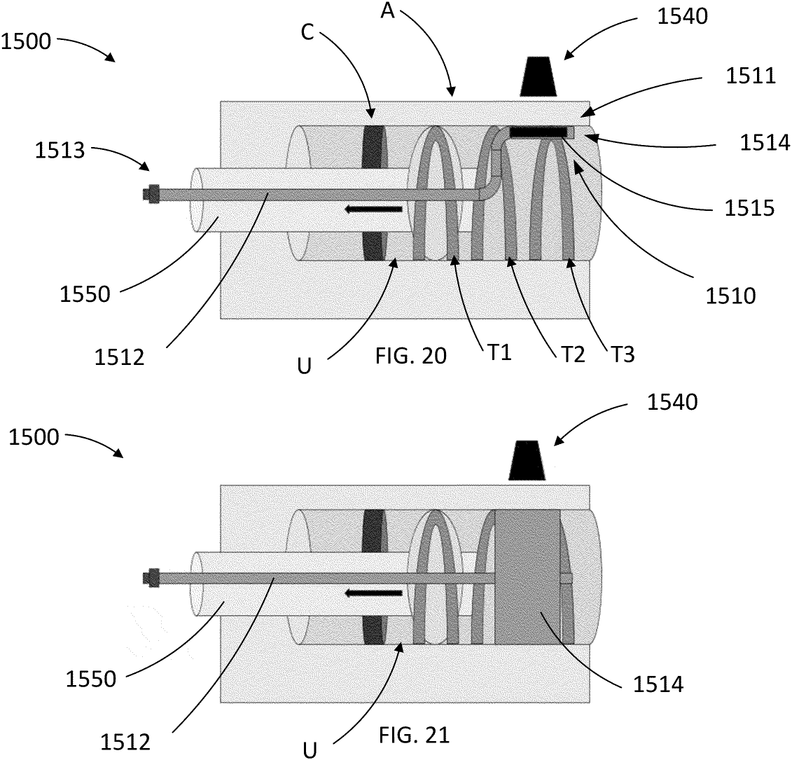

FIG. 20 is a schematic illustration of a system 1500 disposed in an upper trachea U of a patient. The system 1500 includes an inflation assembly 1510. The system 1500 can optionally include a tubular member 1550, an external magnetic assembly 1540, and/or an ultrasound probe (not shown). The inflation assembly 1510 can be similar in structure and/or function to the inflation assembly 110 described above with respect to the system 100. For example, the inflation assembly 1510 can include an elongated tube 1512, an inflatable member 1514, and a magnetic member 1515. The elongated tube 1512 can have a first end 1511 and a second end 1513. In some embodiments, the elongated tube 1512 can have a length sufficient to extend from at least an oral or nasal orifice of a patient to the trachea of the patient. The inflatable member 1514 and the magnetic member 1515 can be coupled to the elongated tube 1512 at or near the first end 1511 of the elongated tube 1512. The inflation assembly 1510 can include an inflation lumen in fluid communication with the inflatable member 1514 such that fluid can be delivered to or drawn from the inflatable member 1514 via the inflation lumen to transition the inflatable member 1514 between an unexpanded configuration (e.g., a uninflated configuration) and an expanded configuration (e.g., an inflated configuration). In some embodiments, the inflation lumen can be disposed within and/or be defined by the elongated tube 1512.

The inflatable member 1514 can be similar in structure and/or function to any of the inflatable members described herein. For example, the inflatable member 1514 can surround the elongated tube 1512 in an expanded and/or unexpanded configuration. In some embodiments, the inflatable member 1514 can extend laterally from the elongated tube 1512 in an expanded and/or unexpanded configuration. In some embodiments, the inflatable member 1514 can extend distally from the first end 1511 of the elongated tube 1512 in an expanded and/or unexpanded configuration. In some embodiments, the inflatable member 1514 can be disposed on the elongated tube 1512 such that a portion of the elongated tube 1512 extends distally of the inflatable member 1514 when the inflatable member 1514 is in the expanded and/or unexpanded configuration. The elongated tube 1512 can define one or more inflation ports or holes such that the inflation lumen can be in fluid communication with the interior of the inflatable member 1514 for transitioning the inflatable member 1514 between the expanded and/or unexpanded configuration.

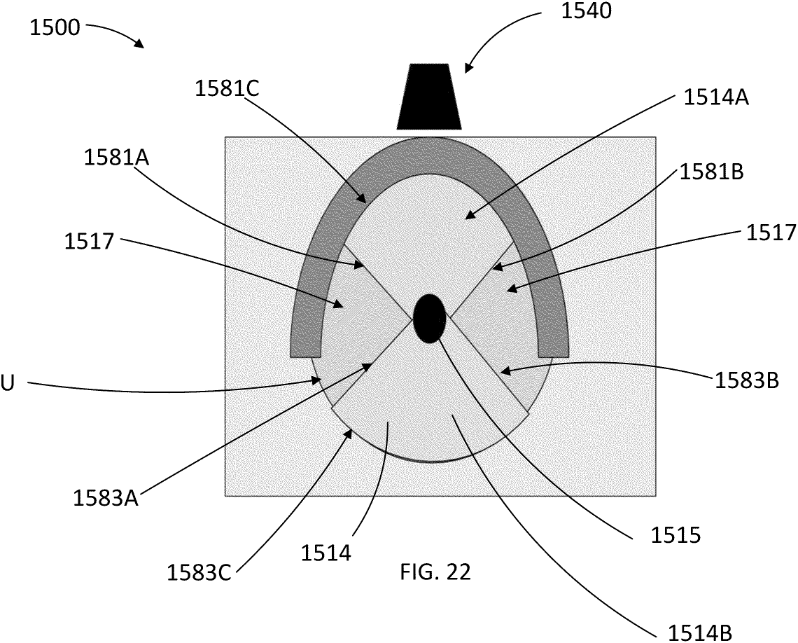

In some embodiments, the inflatable member 1514 can define passageways 1517 (also referred to as “vents”) such that fluid (e.g., air) can travel through the passageways 1517 when the inflatable member 1514 is in the expanded configuration. For example, FIG. 22 is a cross-sectional illustration of the inflatable member 1514 in the expanded configuration. As shown in FIG. 22, the inflatable member 1514 can define a first passageway 1517 and a second passageway 1517. Each of the first passageway 1517 and the second passageway 1517 can be partially bounded by the inflatable member 1514 and partially bounded by the patient’s tracheal wall. Thus, air can flow between a portion of the trachea on a first side of the inflatable member 1514 (e.g., a region between the cricoid ring C and the inflatable member 1514) and a portion of the trachea on a second side of the inflatable member 1514 (e.g., a region between the inflatable member 1514 and the lungs of the patient) through the passageways when the inflatable member 1514 is in the expanded configuration.

As shown in FIG. 22, in some embodiments, the inflatable member 1514 can include a top portion 1514A (also referred to as a first portion) and a bottom portion 1514B (also referred to as a second portion). In the expanded configuration, each of the top portion 1514A and the bottom portion 1514B can be formed as a wedge. For example, the top portion 1514A can have a first side 1581A, a second side 1581B, and an arcuate side 1581C. The bottom portion 1514B can have a first side 1583A, a second side 1583B, and an arcuate side 1583C. The first passageway 1517 can be partially defined by the first side 1581A of the top portion 1514A and a first side 1583A of the bottom portion 1514B. The second passageway 1517 can be partially defined by the second side 1581B of the top portion 1514A and the second side 1583B of the bottom portion 1514B. Although the top portion 1514A and the bottom portion 1514B are referred to herein as the “top portion” and the “bottom portion,” in some embodiments, either of the top portion 1514A or the bottom portion 1514B can be shaped to be disposed in contact with the anterior tracheal wall of the patient. For example, the inflatable member 1514 may be symmetrical about an axis containing a central axis of the elongated tube 1512 in at least one of the expanded configuration and the unexpanded configuration.

Although the inflatable member 1514 is shown in FIG. 22 as defining two passageways 1517, the inflatable member 1514 can be shaped to define any suitable number of passageways 1517 in the expanded configuration (e.g., one, three, four, or more passageways 1517). In some embodiments, the inflatable member 1514 can define a suitable number of passageways 1517 having a suitable size (e.g., having a suitable combined cross-sectional area) such that a patient can be properly ventilated (e.g., freely or via a ventilator) through the passageways 1517 when the inflatable member 1514 is disposed in a trachea of the patient and the outer surface of the inflatable member 1514 contacts the inner wall of the trachea of the patient. For example, the outer surface of the inflatable member 1514 can contact the inner wall of the trachea of the patient in an uninterrupted manner such that air cannot flow between the arcuate sides 1581C and 1583C of the inflatable member 1514 and the tracheal wall or the rate that air can flow between the arcuate sides 1581C and 1583C of the inflatable member 1514 and the tracheal wall is insufficient for proper ventilation and oxygenation. In some embodiments, the combined cross-sectional area of the passageways 1517 can be sufficiently large to allow for between about 40 and about 75 liters per minute of airflow through the passageways 1517. Thus, with the inflatable member 1514 deployed in the trachea, the patient can receive closed and continuous ventilation via the passageways 1517, including during ultrasound visualization of the inflatable member 1514 and/or tissue between the inflatable member 1514 and an ultrasound probe. Although the inflatable member 1514 is shown as defining the boundaries of the passageways 1517 in combination with the patient’s tracheal wall, in some embodiments, the inflatable member 1514 can define the entire outer boundary of one or more of the passageways 1517. In some embodiments, the top portion 1514A and the bottom portion 1514B are in fluidic communication with each other such that the inflation lumen 1516 can be used to provide fluid to both of the top portion 1514A and the bottom portion 1514B. In some embodiments, the top portion 1514A and the bottom portion 1514B are fluidically isolated from each other such that separate inflation lumens and/or separate ports are used to provide fluid to both of the top portion 1514A and the bottom portion 1514B.

In some embodiments, the inflatable member 1514 can be formed in any suitable shape, in any suitable size, and of any suitable material. In some embodiments, the shape can be chosen based on the particular application of the system 1500. For example, the shape of the inflatable member 1514 may be selected to improve ultrasound visualization in particular regions of a patient’s body (e.g., a region other than the upper trachea U). Furthermore, the inflatable member 1514 can be sized for improved engagement and retention between the inflatable member 1514 and any suitable component of a guidewire assembly, such as any of the guidewire assemblies described herein, which may optionally be included in the system 1500. For example, the inflatable member 1514 can be sufficiently pliable such that the inflatable member 1514 (e.g., when inflated) can be punctured (e.g., by a needle) to define a pinhole in the wall of the inflatable member 1514 rather than bursting or tearing as a result of puncture. In some embodiments, the inflatable member 1514 can be formed of, for example, polyurethane, silicone, and/or polyvinyl chloride (PVC). In some embodiments, the inflatable member 1514 can have any suitable material properties, wall thicknesses, and/ or inflated outermost diameters.

The magnetic member 1515 can be any suitable magnetic member configured such that movement of the magnetic member 1515 causes corresponding movement of the first end 1511 of the elongated tube 1512. The magnetic member 1515 can be the same or similar in structure and/or function to any of the magnetic members described herein. The magnetic member 1515 can have any suitable shape. For example, in some embodiments, the magnetic member 1515 can be shaped as an elongated rectangular box. In some embodiments, the magnetic member 1515 can be shaped as a cylinder. In some embodiments, the magnetic member 1515 may be arcuate. In some embodiments, the magnetic member 1515 is coupled directly to the elongated tube 1512. In some embodiments, the magnetic member 1515 is disposed within the inflatable member 1514 and at least partially surrounded by the inflatable member 1514. For example, as shown in FIG. 22, the magnetic member 1515 can be at least partially disposed within the inflatable member 1514 (e.g., within the elongated tube 1512 within the inflatable member 1514). In some embodiments, the magnetic member 1515 is coupled directly to the inflatable member 1514. In some embodiments, the system 1500 includes two or more magnetic members 1515. In some embodiments, at least one magnetic member 1515 can be disposed proximal to the inflatable member 1514 and/or at least one magnetic member 1515 can be disposed distal to the inflatable member 1514. In some embodiments, each magnetic member 1515 of a set of magnetic members 1515 (e.g., two or more magnetic members 1515) can be disposed within, coupled to, disposed distal of, and/or disposed proximal of the inflatable member 1514.

In some embodiments, the magnetic member 1515 may not be included in the system 1500 and the inflatable member 1514 may be shaped and sized such that a first portion of the inflatable member 1514 (e.g., one of the top portion 1514A or the bottom portion 1514B) can be disposed in contact with the anterior tracheal wall and a second portion of the inflatable member 1514 (e.g., the other of the top portion 1514A or the bottom portion 1514B) can be disposed in contact with the posterior tracheal wall in the expanded configuration of the inflatable member 1514 such that the arcuate side 1581C of the top portion 1514A is urged against the anterior tracheal wall due to being constrained by the posterior tracheal wall to apply coaptive pressure against the anterior tracheal wall such that an echogenic window defined by the inflatable member 1514 is visible via ultrasound (e.g., Coaptive Ultrasound). In some embodiments, the magnetic member 1515 may not be included in the system 1500 and the inflatable member 1514 may be shaped and sized such that a first portion of the inflatable member 1514 (e.g., one of the top portion 1514A or the bottom portion 1514B) can be disposed in contact with the anterior tracheal wall and a second portion of the inflatable member 1514 (e.g., the other of the top portion 1514A or the bottom portion 1514B) can be disposed in contact with a portion of the tubular member 1550 in the expanded configuration of the inflatable member 1514 such that the arcuate side 1581C of the top portion 1514A is urged against the anterior tracheal wall due to being constrained by the tubular member 1550 to apply coaptive pressure against the anterior tracheal wall such that an echogenic window defined by the inflatable member 1514 is visible via ultrasound (e.g., Coaptive Ultrasound).

In use, an end of the tubular member 1550 (e.g., an ETT) can be inserted through an orifice of a patient (e.g., a nose or mouth of a patient), through a cricoid ring C of the patient, and to a location near a target region of the upper trachea U of the patient such that an inflatable member and the end of the tubular member 1550 are disposed between the target region of the trachea and the cricoid ring C of the patient. The target region of the upper trachea U can include a desired tracheal puncture site (e.g., a location between the second tracheal ring T2 and the third tracheal ring T3 of the patient or a location between the first tracheal ring T1 and the second tracheal ring T2 of the patient). The inflatable member of the tubular member 155 can be transitioned from an undeployed configuration to an expanded configuration such that the tubular member 155 is stabilized (e.g., resistant to axial movement) relative to the tracheal wall and/or such that the inflatable member sealingly contacts the trachea U of the patient such that air cannot flow between the lungs of the patient and the orifice of the patient except through a lumen of the tubular member 1550. In some embodiments, the tubular member 1550 can be fluidically coupled to a ventilator such that the lungs of the patient can be ventilated by the ventilator via the tubular member 1550.

The first end 1511 of the elongated tube 1512 of the inflation assembly 1510 can be inserted through an orifice of the patient (e.g., a nose or mouth of the patient) and translated through a lumen of the tubular member 1550 such that the first end 1511 of the elongated tube 1512 is extended a particular distance beyond the first end 1511 of the elongated tube 1512. The particular distance can be based on a distance by which the inflatable member 1514 and magnetic member 1515 are disposed beyond the end of the tubular member 1550 and outside the tubular member 1550 inside the upper trachea U. As shown in FIG. 20, an external magnetic assembly 1540 can be placed on the anterior neck A of the patient such that the magnetic member 1514 is urged towards (e.g., into contact with) the anterior wall of the upper trachea U due to magnetic interaction between the external magnetic assembly 1540 and the magnetic member 1515. Thus, the external magnetic assembly 1540 can be used to align and/or orient the inflation assembly 1510 within the upper trachea U.

As shown in FIG. 21, fluid can then be delivered to the inflatable member 1514 via the inflation lumen of the inflation assembly 1510 to transition the inflatable member 1514 from the unexpanded configuration to the expanded configuration. As described with respect to other embodiments herein, the fluid can include a fluid and/or a contrast medium such that the inflatable member 1514 is detectable via imaging (e.g., ultrasound). In the expanded configuration, the inflatable member 1514 can engage portions of the tracheal walls such that the location of the inflatable member 1514 is secured within the upper trachea U via pressure applied by the inflatable member 1514 to the inner surface of the tracheal walls, including the anterior tracheal wall. Thus, in some embodiments, the inflatable member 1514 can be secured within the upper trachea U and/or be disposed in coaptive contact with the anterior tracheal wall to apply coaptive pressure to the anterior tracheal wall due to the magnetic interaction between the external magnetic assembly 1540 and the magnetic member 1515 and/or the engagement of the inflatable member 1514 in an expanded configuration with the inner surface of the tracheal wall due to the inflatable member 1514 being constrained between the anterior tracheal wall and the posterior tracheal wall. As shown in FIG. 22, when the inflatable member 1514 is in the expanded configuration, the passageways 1517 defined by the inflatable member 1514 and the tracheal wall allow ventilation through the upper trachea U. The passageways 1517 can be sufficiently large to allow for proper ventilation of the patient during a portion of the procedure (e.g., during tracheostomy tract formation). In some embodiments, the ultrasound probe can then be used to determine a tracheal puncture site between particular tracheal rings (e.g., between the first and second tracheal rings T1, T2 of the patient P or between the second and the third tracheal rings T2, T3 of the patient P). The inflatable member 1514 can be optionally repositioned as described with respect to any of the other systems described herein. With the inflatable member 1510 properly disposed against the anterior tracheal wall and the tracheal puncture site determined, the inflatable member 1510 can be coupled to a guidewire assembly via operational steps that are the same or similar to the operational steps described above with respect to any of the systems and methods described herein (e.g., the operational steps associated with the system 100 and/or the system 300).

Although the inflation assembly 1510 is shown and described as being delivered to the upper trachea U using an inline approach through the tubular member 1550, in some embodiments the inflation assembly 1510 can be delivered and imaged using a paired approach, similarly as described above with respect to the system 800. In some embodiments, the inflation assembly 1510 can be properly aligned and/or oriented with the anterior tracheal wall of the patient via inserting the inflation assembly 1510 with a particular directional orientation (e.g., with the top portion 1514A directed upward) and maintaining the orientation of the elongated tube 1512 as the inflation assembly 1510 is translated to the upper trachea U. In some embodiments, the orientation of the inflatable member 1514 can be manipulated as needed within the upper trachea U (e.g., based on ultrasound imaging) to properly align the top portion 1514A or the bottom portion 1514B with the anterior tracheal wall via manual manipulation of the elongated tube 1512 (e.g., rotation) and/or via using the external magnetic assembly 1540 to urge the inflation assembly 1510 toward the properly aligned orientation. In some embodiments, the bottom portion 1514B can include or be coupled to a barrier member (not shown) to prevent a needle stick of the posterior tracheal wall through the inflatable member 1514 and the top portion 1514A can be configured to be aligned to contact the anterior tracheal wall. The barrier member may be the same or similar in structure and/or function to the barrier member 195 and/or the barrier member 695 described above with reference to FIG. 1 and FIG. 6, respectively.

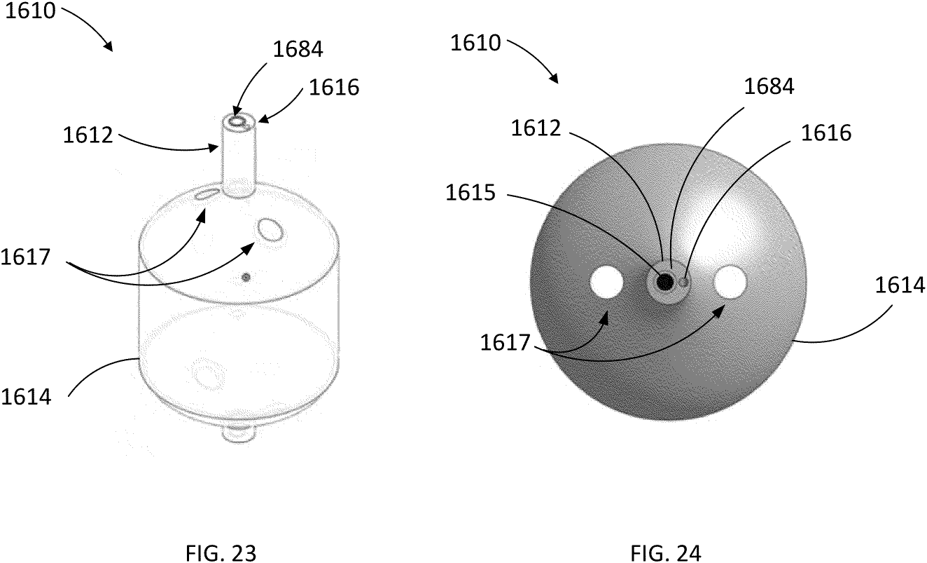

FIGS. 23 through 26 are perspective, front, cross-sectional, and perspective cross-sectional views, respectively, of a portion of an inflation assembly 1610 in an expanded configuration. The inflation assembly 1610 can be similar in structure and/or function to any of the inflation assemblies described herein, such as the inflation assembly 110 of the system 100 and/or the inflation assembly 1510 of the system 1500. For example, the inflation assembly 1610 can include an elongated tube 1612, an inflatable member 1614, and a magnetic member 1615. The elongated tube 1612 defines an inflation lumen 1616 in fluid communication with an interior of the inflatable member 1614 such that fluid can be delivered to and/or drawn from the interior of the inflatable member 1614 to transition the inflatable member 1614 between an unexpanded or undeployed (e.g., uninflated) configuration and an expanded or deployed (e.g. inflated) configuration. As shown, the inflatable member 1614 is disposed on a distal end of the elongated tube 1612, and the elongated tube 1612 includes an inner tubular member 1684 that is disposed within the elongated tube 1612 and within the inflatable member 1614 such that the distal end of the inflatable member 1614 is coupled to the distal end of the inner tubular member 1684. In some embodiments, the inner tubular member 1684 can be more rigid than the elongated tube 1612. The inner tubular member 1684 can define a central lumen within which the magnetic member 1615 can be disposed. In some embodiments, the magnetic member 1615 can be centered between a first end and a second end of the inflatable member 1614.

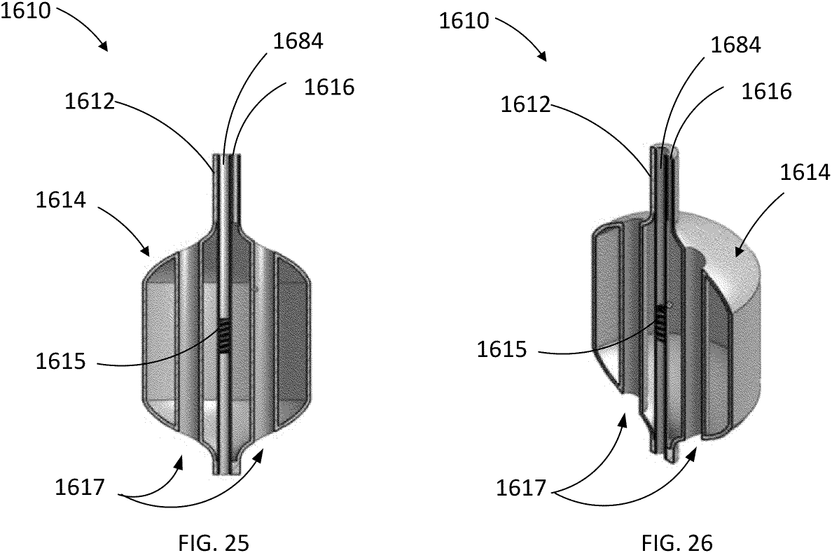

As shown, in FIGS. 25 and 26, the inflatable member 1614 can define two passageways 1617 (also referred to as “vents”) extending through the inflatable member 1614 such that fluid (e.g., air) can pass through the inflatable member 1614 when the inflatable member 1614 is disposed within a trachea of a patient in the expanded configuration. Although FIGS. 23-26 show the inflatable member 1614 as defining two passageways 1617, the inflatable member 1614 can define any suitable number of passageways 1617 (e.g., one, three, four, or more passageways 1617). Although the passageways 1617 are shown as being cylindrical, the passageways 1617 can have any suitable shape. In some embodiments, the inflatable member 1614 can define a suitable number of passageways 1617 having a suitable size (e.g., having a suitable combined cross-sectional area) such that a patient can be properly ventilated (e.g., freely or via a ventilator) through the passageways 1617 when the inflatable member 1614 is disposed in a trachea of the patient and the outer surface of the inflatable member 1614 contacts the inner wall of the trachea of the patient (e.g., in an uninterrupted manner such that air cannot flow along an outer surface of the inflatable member 1614 or the rate that air can flow along an outer surface of the inflatable member 1614 is insufficient for proper ventilation and oxygenation). Although the passageways 1617 are shown as being disposed in a plane including a central axis of the inflatable member 1614 and equidistant from the central axis of the inflatable member 1614, the passageways 1617 can be disposed or defined in any suitable location relative to the central axis of the inflatable member 1614 (e.g., entirely on one side of a plane including the central axis and any suitable distance or distances from the central axis).

In some embodiments, the inflatable member 1614 can be the same or similar in structure and/or function to any of the inflatable members described herein, such as the inflatable member 1514. For example, the inflatable member 1614 can be formed of any suitable material having characteristics described with respect to other inflatable members described herein. In some embodiments, the magnetic member 1615 can be the same or similar in structure and/or function to any of the magnetic members described herein, such as the magnetic member 1515. For example, the magnetic member 1615 can be configured to be urged toward an external magnetic assembly (such as the external magnetic assembly 1540) when disposed in the patient’s upper trachea. Furthermore, although the magnetic member 1615 is shown disposed inside the inner tubular member 1684, the magnetic member 1615 can be coupled to any suitable portion of the inflation assembly 1610 (e.g., an outer surface of the inner tubular member 1684, directly to the inflatable member 1614 such as at the end(s) and/or along an inner or outer surface of the inflatable member 1614, and/or to the elongated tube 1612). Additionally, although the inflation lumen 1616 is shown as being defined by the elongated tube 1612, in some embodiments the inflation lumen can be defined between the inner tubular member 1684 and the elongated tube 1612 such that the inflation lumen surrounds the inner tubular member 1684. In use, the inflation assembly 1610 can be operated the same or similarly as described with respect to other inflation assemblies described herein, such as the inflation assembly 1510.

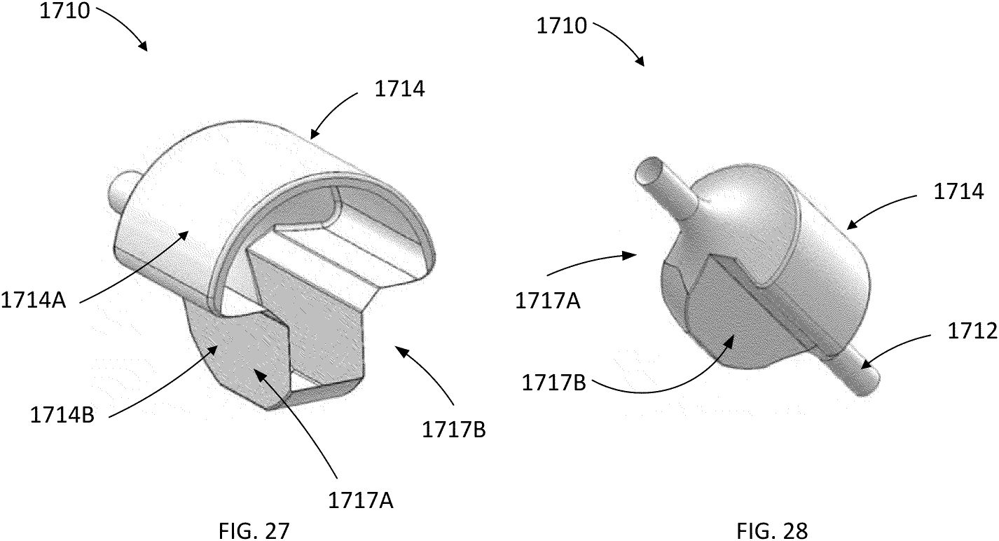

FIGS. 27 through 30 are perspective cross-sectional, side perspective, front perspective, and rear perspective cross-sectional views, respectively, of an inflation assembly 1710 in an expanded configuration. The inflation assembly 1710 can be similar in structure and/or function to any of the inflation assemblies described herein, such as the inflation assembly 110 of system 100 and/or the inflation assembly 1510 of system 1500. For example, the inflation assembly 1710 can include an elongated tube 1712, an inflatable member 1714, and a magnetic member 1715. The elongated tube 1712 can define an inflation lumen (e.g., fully or in combination with another component within the elongated tube 1712 such as an inner tubular member) in fluid communication with the interior of the inflatable member 1714 such that fluid can be delivered to and/or drawn from the interior of the inflatable member 1714 to transition the inflatable member 1714 between an unexpanded or undeployed (e.g., uninflated) configuration and an expanded or deployed (e.g. inflated) configuration. As shown, the inflatable member 1714 can be disposed on or near a distal end of the elongated tube 1712. In some embodiments, although not shown, the elongated tube 1712 can pass through the inflatable member 1714 such that the inflatable member 1714 surrounds a portion of the elongated tube 1712 and a portion of the elongated tube 1712 extends distally of the inflatable member 1714.

As shown in FIGS. 27 and 28, the inflatable member 1714 can be shaped to define a boundary of a first passageway 1717A and a boundary of a second passageway 1717B. For example, the inflatable member 1714 can include a top portion 1714A and a bottom portion 1714B. The top portion 1714A and the bottom portion 1714B can collectively define an inflatable interior of the inflatable member 1714. The top portion 1714A can have, for example, a semi-circular cross section or a wedge-shaped cross-section. The bottom portion 1714B can have, for example, a non-semicircular cross-section (e.g., a substantially rectangular cross-section with the exception of tapered transitional edges as shown in FIGS. 27 and 28 or a wedge-shaped cross-section) such that a first portion of the outer surface of the top portion 1714A and a first portion of the outer surface of the bottom portion 1714B collectively define boundaries of the first passageway 1717A and a second portion of the outer surface of the top portion 1714A and a second portion of the outer surface of the bottom portion 1714B collectively define boundaries of the second passageway 1717B. Thus, when the inflatable member 1714 is in an expanded configuration within a patient’s upper trachea, the top portion 1714 can be disposed against an anterior tracheal wall of the patient, and air can flow through the first passageway 1717A defined by the inflatable member 1714 and a first portion of the patient’s tracheal wall and the second passageway 1717B defined by the inflatable member 1714 and a second portion of the patient’s tracheal wall. In some embodiments, when in the expanded configuration, the inflatable member 1714 can be shaped and sized such that the bottom portion 1714B can contact and be constrained by the posterior tracheal wall, urging the top portion 1714A against the anterior tracheal wall to apply coaptive pressure against the anterior tracheal wall.

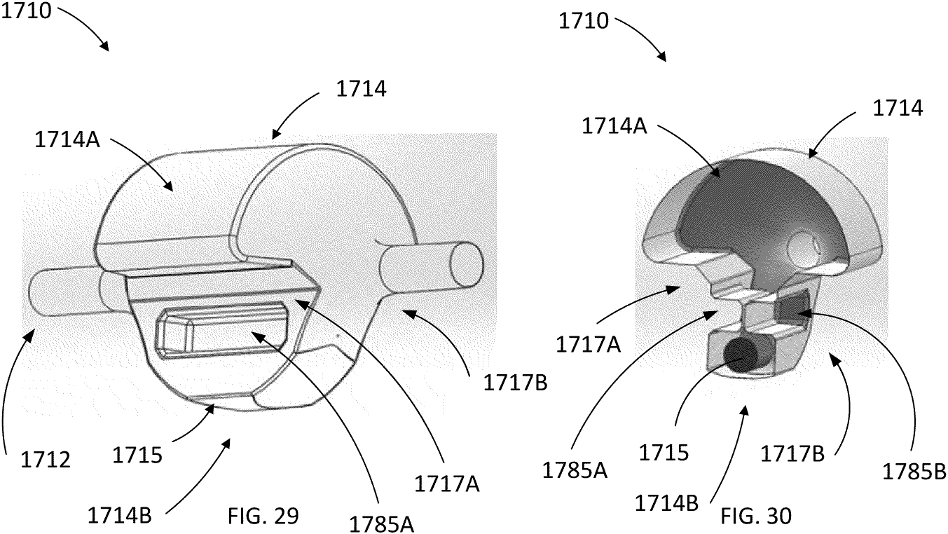

Although not shown in FIGS. 27 and 28, the magnetic member 1715 (or more than one magnetic member 1715) can be coupled to or included in the inflation assembly 1710 in at any suitable location. In some embodiments, the magnetic member 1715 can be optionally not included in the inflation assembly 1710 and coaptation between the inflatable member 1714 and the anterior neck portion of the patient can be achieved under only the pressure of the fluid inside the inflatable member 1714. As shown in FIG. 30, in some embodiments, the magnetic member 1715 can be disposed within the inflatable member 1714 such that magnetic member 1715 is not coaxial with the elongated tube. In some embodiments, the magnetic member 1715 can be secured within the bottom portion 1714B of the inflatable member 1714. For example, in some embodiments, as shown in FIGS. 29 and 30, the bottom portion 1714B of the inflatable member 1714 can define a first recessed portion 1785A and a second recessed portion 1785B on opposite sides of the bottom portion 1714B. The first recessed portion 1785A and the second recessed portion 1785B can be configured to retain the magnetic member 1715 in a compartment defined between the first recessed portion 1785A and the second recessed portion 1785B and the lower surface of the bottom portion 1714B, as shown in FIG. 30. An external magnetic assembly (such as the external magnetic assembly 1540) can be used to orient the magnet 1715 within, for example, the trachea of a patient such that the top portion 1714A of the inflatable member 1714 can be disposed against the anterior tracheal wall.

In some embodiments, the inflatable member 1714 can be the same or similar in structure and/or function to any of the inflatable members described herein, such as the inflatable member 1514. For example, the inflatable member 1714 can be formed of any suitable material having characteristics described with respect to other inflatable members described herein. In some embodiments, the magnetic member 1715 can be the same or similar in structure and/or function to any of the magnetic members described herein, such as the magnetic member 1515. For example, the magnetic member 1715 can be configured to be urged toward an external magnetic assembly (such as the external magnetic assembly 1540) when disposed in the patient’s upper trachea. Furthermore, although the magnetic member 1715 is shown in FIGS. 29 and 30 as being disposed inside a compartment of the inflatable member 1714 defined by the first recessed portion 1785A and the second recessed portion 1785B, the magnetic member 1715 can be coupled to any suitable portion of the inflation assembly 1710 (e.g., an outer surface of the elongated member 1712 inside and/or outside the inflatable member 1714 and/or directly to the inflatable member 1714 such as at the end(s) and/or along an inner or outer surface of the inflatable member 1714). In use, the inflation assembly 1710 can be operated the same or similarly as described with respect to other inflation assemblies described herein, such as the inflation assembly 1510.

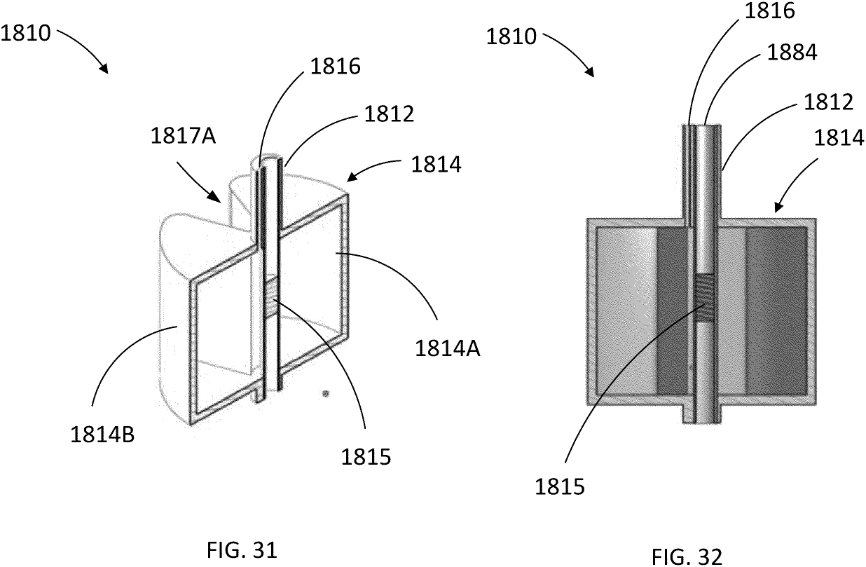

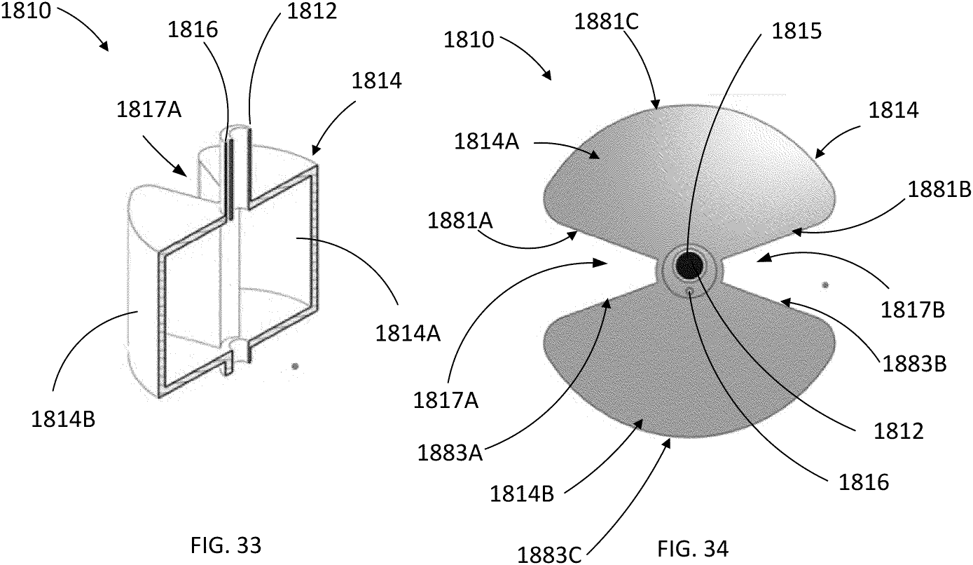

FIGS. 31, 32, and 34 are a perspective cross-sectional, cross-sectional, and end views, respectively, of an inflation assembly 1810 in an expanded configuration. FIG. 33 is a perspective cross-sectional view of the inflation assembly 1810 with an inner tubular member 1884 of the inflation assembly 1810 not shown. The inflation assembly 1810 can be similar in structure and/or function to any of the inflation assembly systems and devices described herein, such as the inflation assembly 110 of system 100 and/or the inflation assembly 1510 of system 1500. For example, the inflation assembly 1810 can include an elongated tube 1812, an inflatable member 1814, and a magnetic member 1815. The elongated tube 1812 defines an inflation lumen 1816 in fluid communication with an interior of the inflatable member 1814 such that fluid can be delivered to and/or drawn from the interior of the inflatable member 1814 to transition the inflatable member 1814 between an unexpanded or undeployed (e.g., uninflated) configuration and the expanded or deployed (e.g. inflated) configuration. As shown, the inflatable member 1814 is disposed on a distal end of the elongated tube 1812, and the elongated tube 1812 includes the inner tubular member 1884 that is disposed within the elongated tube 1812 and within the inflatable member 1814 such that the distal end of the inflatable member 1814 is coupled to the distal end of the inner tubular member 1884. In some embodiments, the inner tubular member 1884 can be more rigid than the elongated tube 1812. The inner tubular member 1884 can define a central lumen within which the magnetic member 1815 can be disposed. In some embodiments, the magnetic member 1815 can be centered between a first end and a second end of the inflatable member 1814.

The inflatable member 1814 defines passageways 1817 (e.g., a first passageway 1817A and a second passageway 1817B) such that fluid (e.g., air) can travel through the passageways 1817 when the inflatable member 1814 is in the expanded configuration. In the expanded configuration, when the inflatable member 1814 is disposed in an upper trachea of a patient, each of the first passageway 1817A and the second passageway 1817B can be partially bounded by the inflatable member 1814 and partially bounded by the patient’s tracheal wall. Thus, air can flow between a portion of the trachea on a first side of the inflatable member 1814 (e.g., a region between a cricoid ring of the patient and the inflatable member 1814) and a portion of the trachea on a second side of the inflatable member 1814 (e.g., a region between the inflatable member 1814 and the lungs of the patient) through the passageways 1817 such that the patient can ventilate through the passageways 1817 and oxygenate when the inflatable member 1814 is in the expanded configuration.

The inflatable member 1814 can include a top portion 1814A (also referred to as a first portion) and a bottom portion 1814B (also referred to as a second portion). In the expanded configuration, each of the top portion 1814A and the bottom portion 1814B can be formed as a wedge. For example, the top portion 1814A can have a first side 1881A, a second side 1881B, and an arcuate side 1881C. The bottom portion 1814B can have a first side 1883A, a second side 1883B, and an arcuate side 1883C. The first passageway 1817A can be partially defined by the first side 1881A of the top portion 1814A and a first side 1883A of the bottom portion 1814B. The second passageway 1817B can be partially defined by the second side 1881B of the top portion 1814A and the second side 1883B of the bottom portion 1814B. Although the top portion 1814A and the bottom portion 1814B are referred to herein as the “top portion” and the “bottom portion,” in some embodiments, either of the top portion 1814A or the bottom portion 1814B can be shaped to be disposed in contact with the anterior tracheal wall of the patient. For example, the inflatable member 1814 may be symmetrical about an axis containing a central axis of the elongated tube 1812 and/or the inner tubular member 1884 in at least one of the expanded configuration and the unexpanded configuration. In some embodiments, when in the expanded configuration, the inflatable member 1814 can be shaped and sized such that the bottom portion 1814B can contact and be constrained by the posterior tracheal wall, urging the top portion 1814A against the anterior tracheal wall to apply coaptive pressure against the anterior tracheal wall.

Although the inflatable member 1814 is shown in FIG. 34 as defining two passageways 1817, the inflatable member 1814 can be shaped to define any suitable number of passageways 1817 in the expanded configuration (e.g., one, three, four, or more passageways 1817). In some embodiments, the inflatable member 1814 can define a suitable number of passageways 1817 having a suitable size (e.g., having a suitable combined cross-sectional area) such that a patient can be properly ventilated (e.g., freely or via a ventilator) through the passageways 1817 when the inflatable member 1814 is disposed in a trachea of the patient and the outer surface of the inflatable member 1814 contacts the inner wall of the trachea of the patient (e.g., in an uninterrupted manner such that air cannot flow along an outer surface of the inflatable member 1814 or the rate that air can flow along an outer surface of the inflatable member 1814 is insufficient for proper ventilation and oxygenation). In some embodiments, the top portion 1814A and the bottom portion 1814B are in fluidic communication with each other such that the inflation lumen 1816 can be used to provide fluid to both of the top portion 1814A and the bottom portion 1814B. In some embodiments, the top portion 1814A and the bottom portion 1814B are fluidically isolated from each other such that separate inflation lumens and/or separate ports are used to provide fluid to both of the top portion 1814A and the bottom portion 1814B.

In some embodiments, the inflatable member 1814 can be the same or similar in structure and/or function to any of the inflatable members described herein, such as the inflatable member 1514. For example, the inflatable member 1814 can be formed of any suitable material having characteristics described with respect to other inflatable members described herein. In some embodiments, the magnetic member 1815 can be the same or similar in structure and/or function to any of the magnetic members described herein, such as the magnetic member 1815. For example, the magnetic member 1815 can be configured to be urged toward an external magnetic assembly (such as the external magnetic assembly 1540) when disposed in the patient’s upper trachea. Furthermore, although the magnetic member 1815 is shown disposed inside the inner tubular member 1884, the magnetic member 1815 can be coupled to any suitable portion of the inflation assembly 1810 (e.g., an outer surface of the inner tubular member 1884, directly to the inflatable member 1814 such as at the end(s) and/or along an inner or outer surface of the inflatable member 1814, and/or to the elongated tube 1812). In some embodiments, the magnetic member 1815 can be optionally not included in the inflation assembly 1810 and coaptation between the inflatable member 1814 and the anterior neck portion of the patient can be achieved under only the pressure of the fluid inside the inflatable member 1814. Additionally, although the inflation lumen 1816 is shown as being defined by the elongated tube 1812, in some embodiments the inflation lumen can be defined between the inner tubular member 1884 and the elongated tube 1812 such that the inflation lumen surrounds the inner tubular member 1884. In use, the inflation assembly 1810 can be operated the same or similarly as described with respect to other inflation assemblies described herein, such as the inflation assembly 1510.

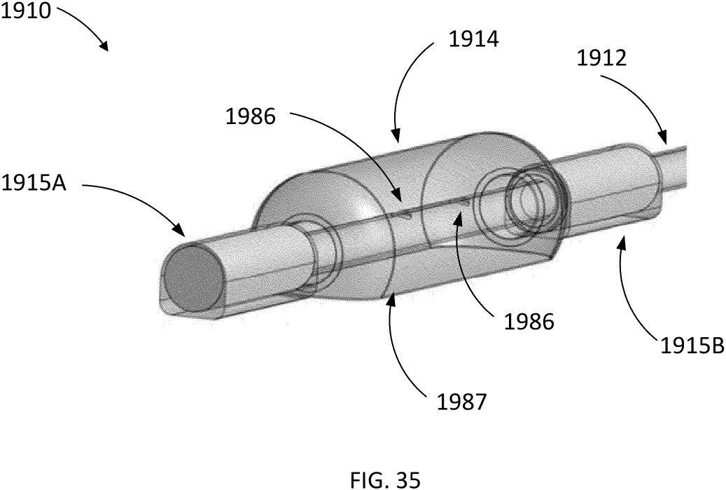

FIGS. 35 through 37 are front perspective, perspective cross-section, and side views, respectively, of an inflation assembly 1910. The inflation assembly 1910 can be the same or similar in structure and/or function to any of the inflation systems or devices described herein, such as the inflation assembly 110, the inflation assembly 410, and/or the inflation assembly 1510 described above. For example, the inflation assembly 1910 can include an elongated tube 1912, an inflatable member 1914, a first magnetic member 1915A, and a second magnetic member 1915B. The elongated tube 1912 defines an inflation lumen in fluid communication with an anterior of the inflatable member 1914 such that fluid can be delivered to and/or drawn from the interior of the inflatable member 1914 to transition the inflatable member 1914 between an unexpanded or undeployed (e.g., uninflated) configuration and an expanded or deployed (e.g. inflated) configuration. As shown, the inflatable member 1914 is disposed near a distal end of the elongated tube 1912, and the elongated tube 1912 extends through the inflatable member 1914 such that a portion of the elongated tube 1912 extends distally of the inflatable member 1914. As shown in FIG. 35, the elongated member 1912 can include a number of ports 1986 in fluidic communication with the inflation lumen such that fluid can be transferred to and/or from the interior of the inflatable member 1914 via the ports 1986.

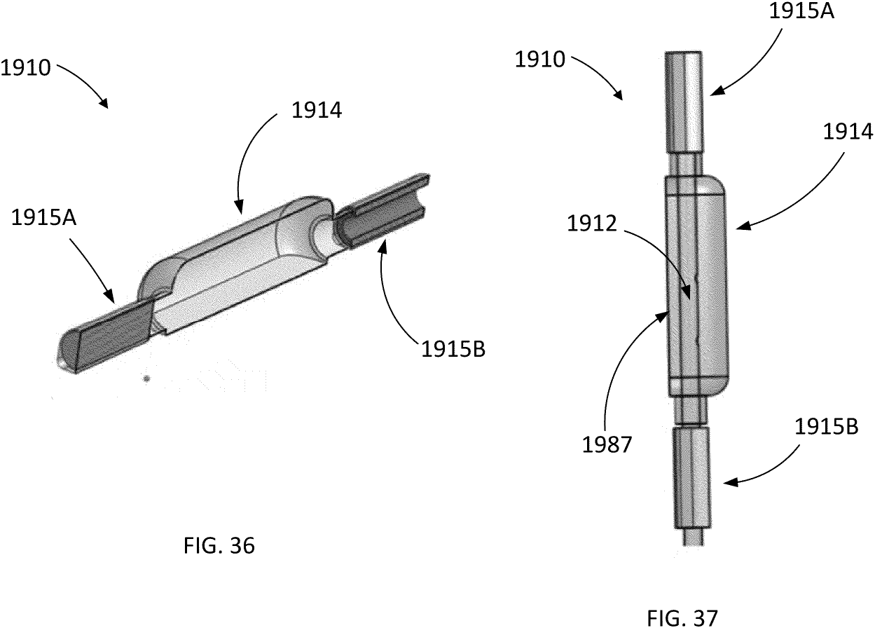

The first magnetic member 1915A can be coupled to (e.g., disposed on or inside a lumen of) the portion of the elongated tube 1912 extending distally of the inflatable member 1914. The second magnetic member 1915B can be coupled to (e.g., disposed on or inside a lumen of) a portion of the elongated tube 1912 proximal of the inflatable member 1914. In some embodiments, the first magnetic member 1915A and the second magnetic member 1915B can be disposed adjacent opposing ends of the inflatable member 1914 and about equidistant from opposing ends of the inflatable member 1914. As shown in FIG. 36, the second magnetic member 1915B can define a lumen along a central axis of the second magnetic member 1915B, the lumen in fluid communication with an inflation lumen of the elongated tube such that fluid can be transferred to and/or from the interior of the inflatable member 1914 via the lumen of the second magnetic member 1915B.

The inflatable member 1914 can be formed having any suitable shape having a passageway feature such that fluid (e.g., air) can pass through the inflatable member 1914 when the inflatable member 1914 is disposed within a trachea of a patient in the expanded configuration. For example, as shown in FIGS. 35 and 37, the inflatable member 1914 can include a flat surface 1987 extending from a first end to a second end of the inflatable member 1914. A remainder of the outer surface of the inflatable member 1914 can be formed as a partial cylinder in the expanded configuration of the inflatable member 1914. In some embodiments, when oriented inside a trachea of a patient, the flat surface 1987 can be arranged opposite a surface of the inflatable member 1914 that is configured to contact an anterior tracheal wall of the patient. In some embodiments, when oriented inside a trachea of a patient, without the flat surface 1987 contacting the anterior tracheal wall of the patient, the flat surface 1987 can be arranged at an angle relative to a surface of the inflatable member 1914 that is configured to contact an anterior tracheal wall of the patient. Thus, since the inflatable member 1914 does not expand to form a full cylindrical shape, the flat surface 1987 can collectively define a passageway through the trachea of the patient in combination with a surface of the patient’s tracheal wall. The flat surface 1987 can be sufficiently wide such that the passageway has a cross-sectional area sufficiently large for the patient to be properly ventilated and oxygenated when the inflatable member 1914 is disposed in the patient’s trachea in the expanded configuration.

In some embodiments, the inflatable member 1914 can be the same or similar in structure and/or function to any of the inflatable members described herein, such as the inflatable member 1514. For example, the inflatable member 1914 can be formed of any suitable material having characteristics described with respect to other inflatable members described herein. In some embodiments, each of the first magnetic member 1915A and the second magnetic member 1915B can be the same or similar in structure and/or function to any of the magnetic members described herein, such as the magnetic member 1515. For example, each of the first magnetic member 1915A and the second magnetic member 1915B can be configured to be urged toward an external magnetic assembly (such as the external magnetic assembly 1540) when disposed in the patient’s upper trachea. Furthermore, although the each of the first magnetic member 1915A and the second magnetic member 1915B are shown disposed outside of the inflatable member 1914, the first magnetic member 1915A and/or the second magnetic member 1915B can be disposed inside of the inflatable member 1914 and/or coupled to an outside of the inflatable member 1914. In use, the inflation assembly 1610 can be operated the same or similarly as described with respect to other inflation assemblies described herein, such as the inflation assembly 1510.

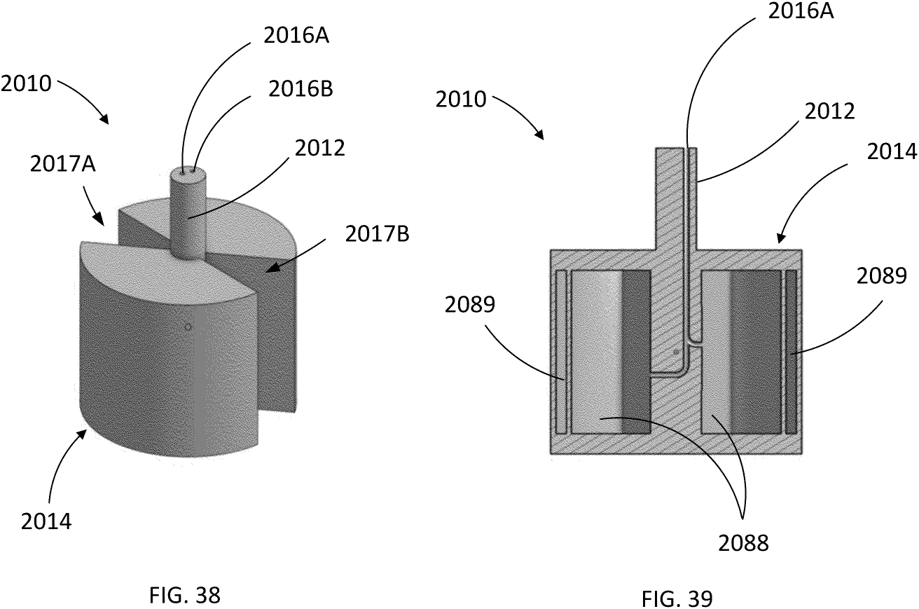

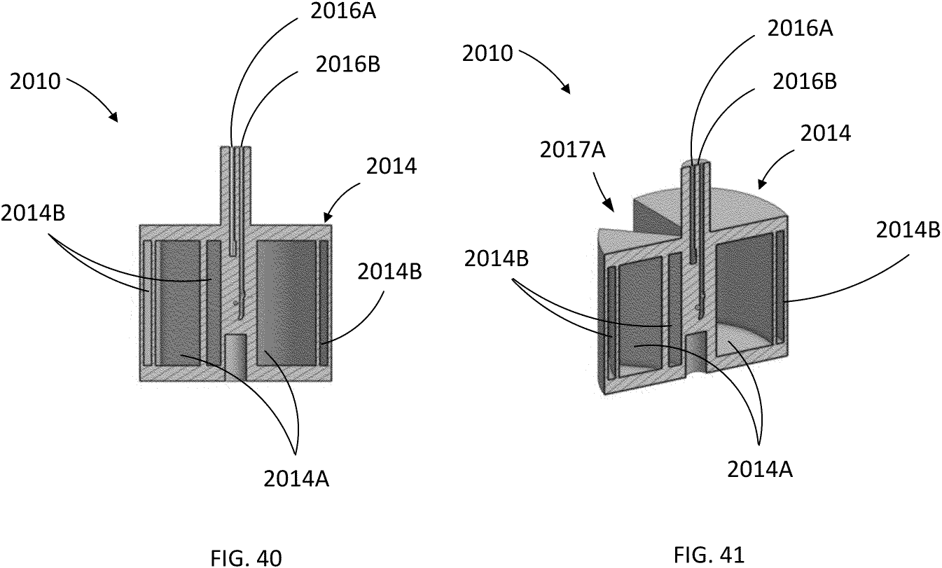

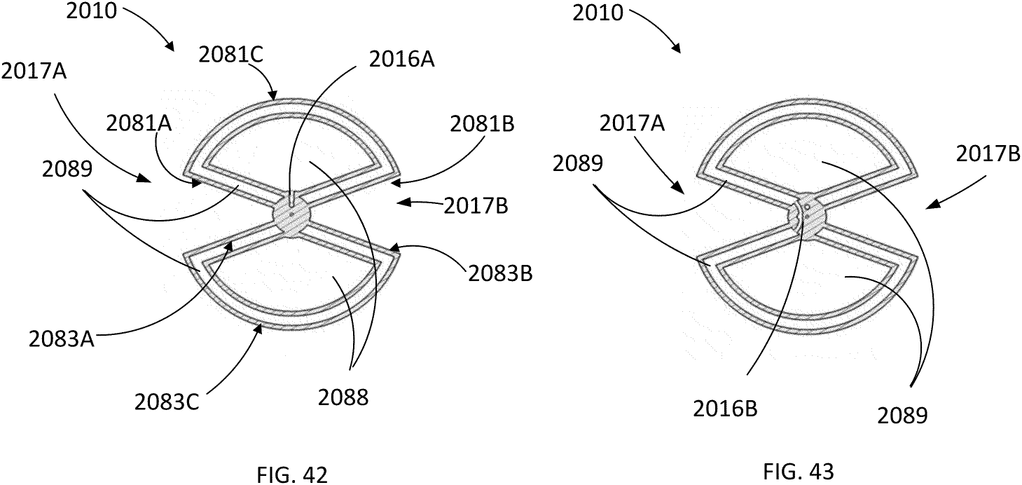

FIGS. 38 through 43 are a perspective, first side cross-sectional, second side cross-sectional, perspective cross-sectional, first top cross-sectional, and second top cross-sectional views, respectively, of an inflation assembly 2010. The inflation assembly 110 can be similar in structure and/or function to any of the inflation assembly systems and devices described herein, such as the inflation assembly 110 of system 100 and/or the inflation assembly 1510 of system 1500. For example, the inflation assembly 2010 can include an elongated tube 2012 and an inflatable member 2014. Although not shown, the inflation assembly 2010 can include one or more magnetic members that are the same or similar in structure and/or function to nay of the magnetic members described herein. The elongated tube 2012 defines a first inflation lumen 2016A and a second inflation lumen 2016B. The inflatable member 2014 defines a first interior portion 2088 and a second interior portion 2089. The second interior portion 2089 can surround the first interior portion 2088 such that, when the inflatable member 2014 is disposed in contact with a surface such as a tracheal wall, the first interior portion 2088 is separated from the surface by the second interior portion 2089. The first inflation lumen 2016A can be in fluid communication with the first interior portion 2088 such that fluid can be delivered to and/or drawn from the first interior portion 2088 of the inflatable member 2014 to transition the inflatable member 2014 between an unexpanded or undeployed (e.g., uninflated) configuration and an expanded or deployed (e.g. inflated) configuration. The second inflation lumen 2016B can be in fluid communication with the second interior portion 2089 such that fluid can be delivered to and/or drawn from the second interior portion 2089 of the inflatable member 2014. Thus, the first interior portion 2088 can be inflated (e.g., with air) to expand the inflatable member 2014, and the second interior portion 2088 can be filled with a liquid fluid and/or contrast medium such that the second interior portion 2088 is detectable via imaging (e.g., ultrasound). In some embodiments, the air and/or liquid fluid that flows through the first inflation lumen 2016A and the second inflation lumen 2016B, respectively, are sufficiently different and/or provided at sufficiently different volumes and/or pressures such that the first interior portion 2088 and the second interior portion 2089 have different internal pressures. For example, the first interior portion 2088 can be inflated to a greater internal pressure than the second interior portion 2089 such that the first interior portion 2088 provides stiffness to the shape of the inflatable member 2014 but the first interior portion 2089 is more malleable, or compliant, for more consistent engagement with an anterior tracheal wall of the patient.

The inflatable member 2014 defines passageways 2017 (e.g., a first passageway 2017A and a second passageway 2017B) such that fluid (e.g., air) can travel through the passageways 2017 when the inflatable member 2014 is in the expanded configuration. In the expanded configuration, when the inflatable member 2014 is disposed in an upper trachea of a patient, each of the first passageway 2017A and the second passageway 2017B can be partially bounded by the inflatable member 2014 and partially bounded by the patient’s tracheal wall. Thus, air can flow between a portion of the trachea on a first side of the inflatable member 2014 (e.g., a region between a cricoid ring of the patient and the inflatable member 2014) and a portion of the trachea on a second side of the inflatable member 2014 (e.g., a region between the inflatable member 2014 and the lungs of the patient) through the passageways 2017 such that the patient can ventilate through the passageways 2017 and oxygenate when the inflatable member 2014 is in the expanded configuration.

Similar to the inflatable member 1814, for example, the inflatable member 2014 can include a top portion 2014A (also referred to as a first portion) and a bottom portion 2014B (also referred to as a second portion). In the expanded configuration, each of the top portion 2014A and the bottom portion 2014B can be formed as a wedge that includes a portion of the first interior portion 2088 and a portion of the second interior portion 2089. For example, the top portion 2014A can have a first side 2081A, a second side 2081B, and an arcuate side 2081C. The bottom portion 2014B can have a first side 2083A, a second side 2083B, and an arcuate side 2083C. The first passageway 2017A can be partially defined by the first side 2081A of the top portion 2014A and a first side 2083A of the bottom portion 2014B. The second passageway 2017B can be partially defined by the second side 2081B of the top portion 2014A and the second side 2083B of the bottom portion 2014B. Although the top portion 2014A and the bottom portion 2014B are referred to herein as the “top portion” and the “bottom portion,” in some embodiments, either of the top portion 2014A or the bottom portion 2014B can be shaped to be disposed in contact with the anterior tracheal wall of the patient. For example, the inflatable member 2014 may be symmetrical about an axis containing a central axis of the elongated tube 2012 in at least one of the expanded configuration and the unexpanded configuration. In some embodiments, when in the expanded configuration, the inflatable member 2014 can be shaped and sized such that the bottom portion 2014B can contact and be constrained by the posterior tracheal wall, urging the top portion 2014A against the anterior tracheal wall to apply coaptive pressure against the anterior tracheal wall.

Although the inflatable member 2014 is shown in FIG. 38 as defining two passageways 2017, the inflatable member 2014 can be shaped to define any suitable number of passageways 2017 in the expanded configuration (e.g., one, three, four, or more passageways 2017). In some embodiments, the inflatable member 2014 can define a suitable number of passageways 2017 having a suitable size (e.g., having a suitable combined cross-sectional area) such that a patient can be properly ventilated (e.g., freely or via a ventilator) through the passageways 2017 when the inflatable member 2014 is disposed in a trachea of the patient and the outer surface of the inflatable member 2014 contacts the inner wall of the trachea of the patient (e.g., in an uninterrupted manner such that air cannot flow along an outer surface of the inflatable member 2014 or the rate that air can flow along an outer surface of the inflatable member 2014 is insufficient for proper ventilation and oxygenation). In some embodiments, the portion of the first interior portion 2088 of the top portion 2014A and the portion of the first interior portion 2088 of the bottom portion 2014B are in fluidic communication with each other (e.g., directly or via the inflation lumen 2016A) such that the first inflation lumen 2016 A can be used to provide fluid to both portions of the first interior portion 2088. In some embodiments, the portions of the first interior portion 2088 are fluidically isolated from each other such that separate inflation lumens and/or separate ports are used to provide fluid to both of the portions of the first interior portion 2088. In some embodiments, the portion of the second interior portion 2089 of the top portion 2014A and the portion of the second interior portion 2089 of the bottom portion 2014B are in fluidic communication with each other (e.g., directly or via the second inflation lumen 2016B) such that the second inflation lumen 2016B can be used to provide fluid to both portions of the second interior portion 2089. In some embodiments, the portions of the second interior portion 2089 are fluidically isolated from each other such that separate inflation lumens and/or separate ports are used to provide fluid to both of the portions of the second interior portion 2089.

In some embodiments, the inflatable member 2014 can be the same or similar in structure and/or function to any of the inflatable members described herein, such as the inflatable member 1514. For example, the inflatable member 2014 can be formed of any suitable material having characteristics described with respect to other inflatable members described herein. In some embodiments, coaptation between the inflatable member 2014 and the anterior neck portion of the patient can be achieved under only the pressure of the fluid inside the inflatable member 2014. Additionally, although the first inflation lumen 2016A and the second inflation lumen 2016B are shown as being defined by the elongated tube 2012, in some embodiments the first inflation lumen 2016A and/or the second inflation lumen can be defined between another component (e.g., an inner tubular member such as the inner tubular member 1884) and the elongated tube 2012 or by another tube disposed within the elongated tube 2012. In use, the inflation assembly 2010 can be operated the same or similarly as described with respect to other inflation assemblies described herein, such as the inflation assembly 1510.

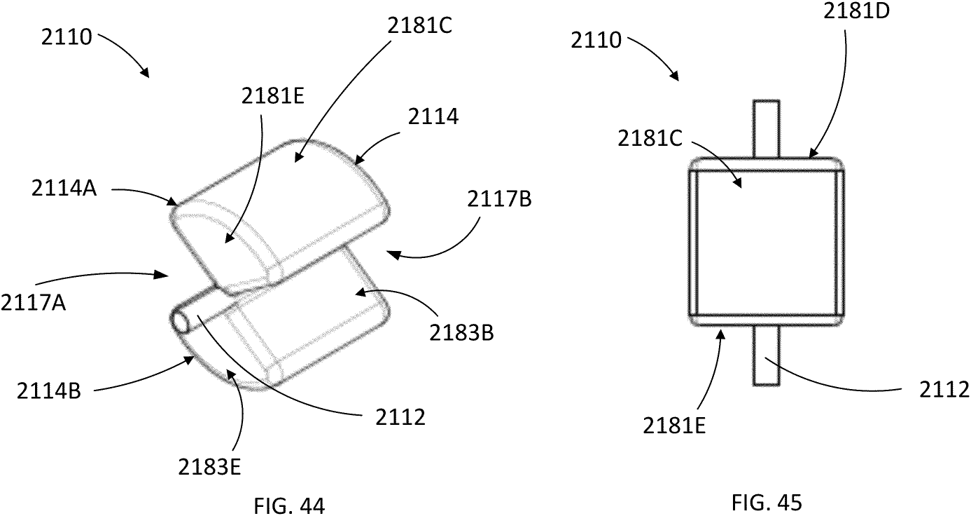

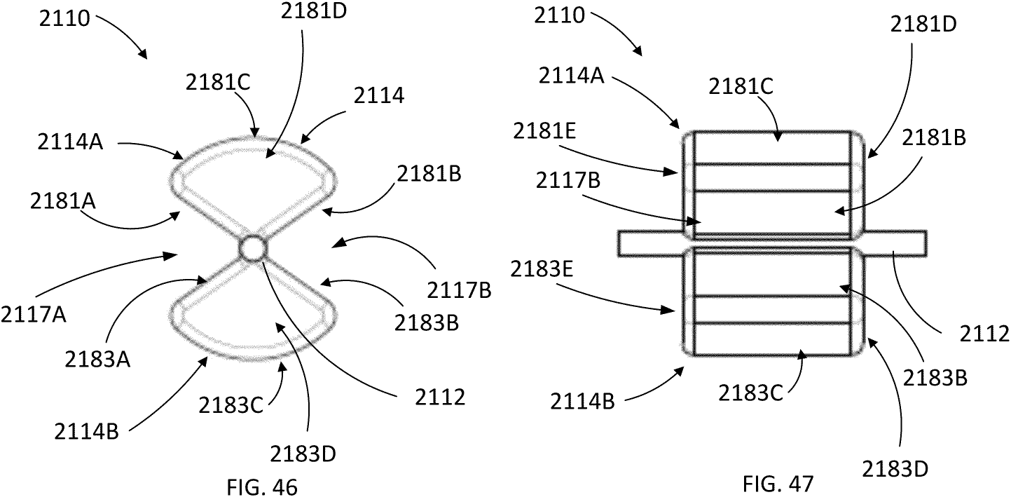

FIGS. 44-47 are a perspective view, a top view, a distal end view, and a side view, respectively, of an inflation assembly 2110 in an expanded configuration. The inflation assembly 2110 can be similar in structure and/or function to any of the inflation assembly systems and devices described herein, such as the inflation assembly 110 of system 100, the inflation assembly 1510 of system 1500, and/or the inflation assembly 1810. For example, the inflation assembly 2110 can include an elongated tube 2112 and an inflatable member 2114. The elongated tube 2112 can define an inflation lumen in fluid communication with an interior of the inflatable member 2114 such that fluid can be delivered to and/or drawn from the interior of the inflatable member 2114 to transition the inflatable member 2114 between an unexpanded or undeployed (e.g., uninflated) configuration and the expanded or deployed (e.g. inflated) configuration. The inflatable member 2114 can be disposed along a portion of the elongated tube 2112 (e.g., near a distal end of the elongated tube 2112).

The inflatable member 2114 defines passageways 2117 (e.g., a first passageway 2117A and a second passageway 2117B) such that fluid (e.g., air) can travel through the passageways 2117 when the inflatable member 2114 is in the expanded configuration. In the expanded configuration, when the inflatable member 2114 is disposed in an upper trachea of a patient, each of the first passageway 2117A and the second passageway 2117B can be partially bounded by the inflatable member 2114 and partially bounded by the patient’s tracheal wall. Thus, air can flow between a portion of the trachea on a first side of the inflatable member 2114 (e.g., a region between a cricoid ring of the patient and the inflatable member 2114) and a portion of the trachea on a second side of the inflatable member 2114 (e.g., a region between the inflatable member 2114 and the lungs of the patient) through the passageways 2117 such that the patient can ventilate through the passageways 2117 and oxygenate when the inflatable member 2114 is in the expanded configuration.

The inflatable member 2114 can include a top portion 2114A (also referred to as a first portion) and a bottom portion 2114B (also referred to as a second portion). In the expanded configuration, each of the top portion 2114A and the bottom portion 2114B can be formed as a wedge. For example, the top portion 2114A can have a first side 2181A, a second side 2181B, and an arcuate side 2181C. The top portion 2114A can also have a distal end 2181D and a proximal end 2181E. The bottom portion 2114B can have a first side 2183A, a second side 2183B, and an arcuate side 2183C. The bottom portion 2114B can also have a distal end 2183D and a proximal end 2183E. The first passageway 2117A can be partially defined by the first side 2181A of the top portion 2114A and a first side 2183A of the bottom portion 2114B. The second passageway 2117B can be partially defined by the second side 2181B of the top portion 2114A and the second side 2183B of the bottom portion 2114B. Although the top portion 2114A and the bottom portion 2114B are referred to herein as the “top portion” and the “bottom portion,” in some embodiments, either of the top portion 2114A or the bottom portion 2114B can be shaped to be disposed in contact with the anterior tracheal wall of the patient. For example, the inflatable member 2114 may be symmetrical about an axis containing a central axis of the elongated tube 2112 in at least one of the expanded configuration and the unexpanded configuration. In some embodiments, when in the expanded configuration, the inflatable member 2114 can be shaped and sized such that the bottom portion 2114B can contact and be constrained by the posterior tracheal wall, urging the top portion 2114A against the anterior tracheal wall to apply coaptive pressure against the anterior tracheal wall.

In some embodiments, the top portion 2114A and the bottom portion 2114B are in fluidic communication with each other such that the inflation lumen of the elongated tube 2112 can be used to provide fluid to both of the top portion 2114A and the bottom portion 2114B. In some embodiments, the top portion 2114A and the bottom portion 2114B are fluidically isolated from each other such that separate inflation lumens and/or separate ports of the elongated tube 2112 are used to provide fluid to the top portion 2114A and the bottom portion 2114B.

In some embodiments, the inflatable member 2114 can be the same or similar in structure and/or function to any of the inflatable members described herein, such as the inflatable member 1514. For example, the inflatable member 2114 can be formed of any suitable material having characteristics described with respect to other inflatable members described herein. The inflation assembly 2110 may also include a magnetic member (not shown) that can be the same or similar in structure and/or function to any of the magnetic members described herein. For example, in some embodiments, the elongated member 2112 can define a central lumen within which the magnetic member can be disposed. In some embodiments, the magnetic member can be centered between a first end and a second end of the inflatable member 2114. In some embodiments, the magnetic member can be the same or similar in structure and/or function to any of the magnetic members described herein, such as the magnetic member 1815. For example, the magnetic member can be configured to be urged toward an external magnetic assembly (such as the external magnetic assembly 1540) when disposed in the patient’s upper trachea. Furthermore, in some embodiments, the magnetic member can be coupled to any suitable portion of the inflation assembly 2110 (e.g., an outer surface of an inner tubular member disposed within the elongated tube 2112, directly to the inflatable member 2114 such as at the end(s) and/or along an inner or outer surface of the inflatable member 2114, and/or to the elongated tube 2112). In some embodiments, the magnetic member can be optionally not included in the inflation assembly 2110 and coaptation between the inflatable member 2114 and the anterior neck portion of the patient can be achieved under only the pressure of the fluid inside the inflatable member 2114. Additionally, in some embodiments the inflation lumen can be defined between an inner tubular member (e.g., inner tubular member 1684) and the elongated tube 2112 such that the inflation lumen surrounds the inner tubular member. In use, the inflation assembly 2110 can be operated the same or similarly as described with respect to other inflation assemblies described herein, such as the inflation assembly 1510 and the inflation assembly 1810. For example, when a portion of the inflatable member 2114 is in coaptive contact with the anterior tracheal wall of the patient, the inflatable member 2114 can be visualized (e.g. via ultrasound) and a needle can pierce the inflatable member 2114 such that a procedure the same or similar as described with respect to FIGS. 3A-3M and/or FIGS. 18-22 can be performed.

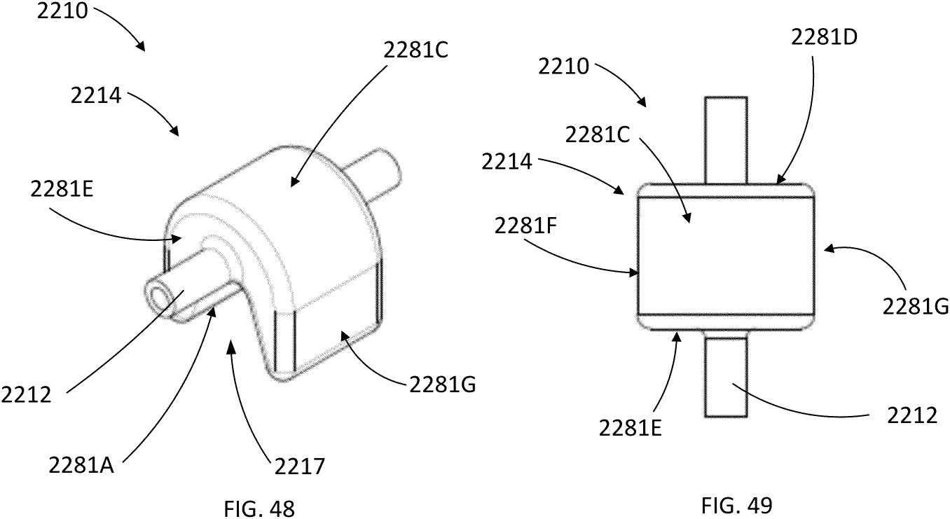

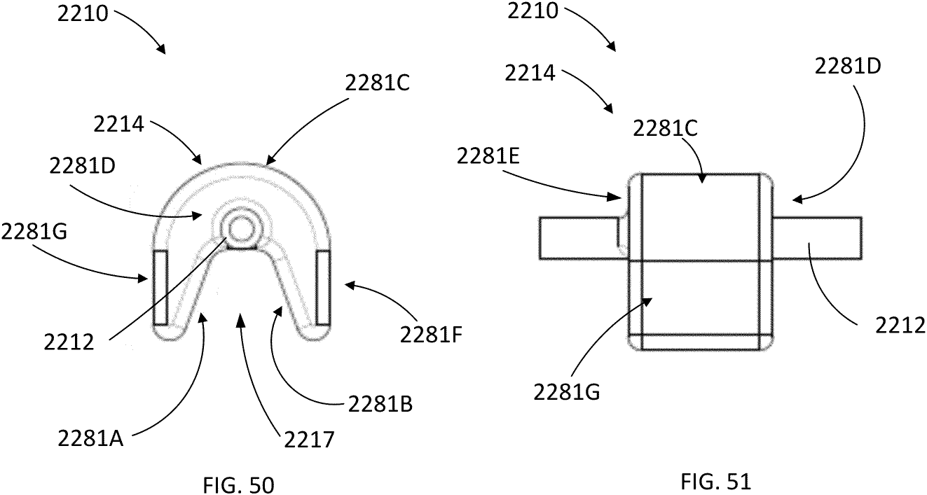

FIGS. 48-51 are a perspective view, a top view, a distal end view, and a side view, respectively, of an inflation assembly 2210 in an expanded configuration. The inflation assembly 2210 can be similar in structure and/or function to any of the inflation assembly systems and devices described herein, such as the inflation assembly 110 of system 100 and/or the inflation assembly 1510 of system 1500. For example, the inflation assembly 2210 can include an elongated tube 2212 and an inflatable member 2214. The elongated tube 2212 can define an inflation lumen in fluid communication with an interior of the inflatable member 2214 such that fluid can be delivered to and/or drawn from the interior of the inflatable member 2214 to transition the inflatable member 2214 between an unexpanded or undeployed (e.g., uninflated) configuration and the expanded or deployed (e.g. inflated) configuration. The inflatable member 2214 can be disposed along a portion of the elongated tube 2212 (e.g., near or at a distal end of the elongated tube 2212).

The inflatable member 2214 defines a passageway 2217 such that fluid (e.g., air) can travel through the passageway 2217 when the inflatable member 2214 is in the expanded configuration. In the expanded configuration, when the inflatable member 2214 is disposed in an upper trachea of a patient, the passageway 2217 can be partially bounded by the inflatable member 2214 and partially bounded by the patient’s tracheal wall. Thus, air can flow between a portion of the trachea on a first side of the inflatable member 2214 (e.g., a region between a cricoid ring of the patient and the inflatable member 2214) and a portion of the trachea on a second side of the inflatable member 2214 (e.g., a region between the inflatable member 2214 and the lungs of the patient) through the passageway 2217 such that the patient can ventilate through the passageway 2217 and oxygenate when the inflatable member 2214 is in the expanded configuration.

In the expanded configuration, the inflatable member 2214 can be formed having a horseshoe-shape. For example, the inflatable member 2214 can have an arcuate top side 2281C, a first side 2281F, and a second side 2281G. The inflatable member 2214 can also have a distal end 2281D and a proximal end 2281E. The inflatable member 2214 can also have a first bottom side 2281A and a second bottom side 2281B. The passageway 2217 can be partially defined by the first bottom side 2281A and the second bottom side 2281B. In some embodiments, when in the expanded configuration, the inflatable member 2214 can be shaped and sized such that the a bottom portion of the inflatable member 2214 (e.g., a portion at the intersection of the first side 2281F and the second bottom side 2281B and a portion at the intersection of the second side 2281G and the first bottom side 2281A) can contact and be constrained by the posterior tracheal wall, urging the arcuate top side 2281C against the anterior tracheal wall to apply coaptive pressure against the anterior tracheal wall.

In some embodiments, the inflatable member 2214 can be the same or similar in structure and/or function to any of the inflatable members described herein, such as the inflatable member 1514. For example, the inflatable member 2214 can be formed of any suitable material having characteristics described with respect to other inflatable members described herein. The inflation assembly 2210 may also include a magnetic member (not shown) that can be the same or similar in structure and/or function to any of the magnetic members described herein. For example, in some embodiments, the elongated member 2212 can define a central lumen within which the magnetic member can be disposed. In some embodiments, the magnetic member can be centered between a first end and a second end of the inflatable member 2214. In some embodiments, the magnetic member can be the same or similar in structure and/or function to any of the magnetic members described herein, such as the magnetic member 1815. For example, the magnetic member can be configured to be urged toward an external magnetic assembly (such as the external magnetic assembly 1540) when disposed in the patient’s upper trachea. Furthermore, in some embodiments, the magnetic member can be coupled to any suitable portion of the inflation assembly 2210 (e.g., an outer surface of an inner tubular member disposed within the elongated tube 2212, directly to the inflatable member 2214 such as at the end(s) and/or along an inner or outer surface of the inflatable member 2214, and/or to the elongated tube 2212). In some embodiments, the magnetic member can be optionally not included in the inflation assembly 2210 and coaptation between the inflatable member 2214 and the anterior neck portion of the patient can be achieved under only the pressure of the fluid inside the inflatable member 2214. Additionally, in some embodiments the inflation lumen can be defined between an inner tubular member (e.g., inner tubular member 1684) and the elongated tube 2212 such that the inflation lumen surrounds the inner tubular member. In use, the inflation assembly 2210 can be operated the same or similarly as described with respect to other inflation assemblies described herein, such as the inflation assembly 1510 and the inflation assembly 1810. For example, when a portion of the inflatable member 2214 is in coaptive contact with the anterior tracheal wall of the patient, the inflatable member 2214 can be visualized (e.g. via ultrasound) and a needle can pierce the inflatable member 2214 such that a procedure the same or similar as described with respect to FIGS. 3A-3M and/or FIGS. 18-22 can be performed.

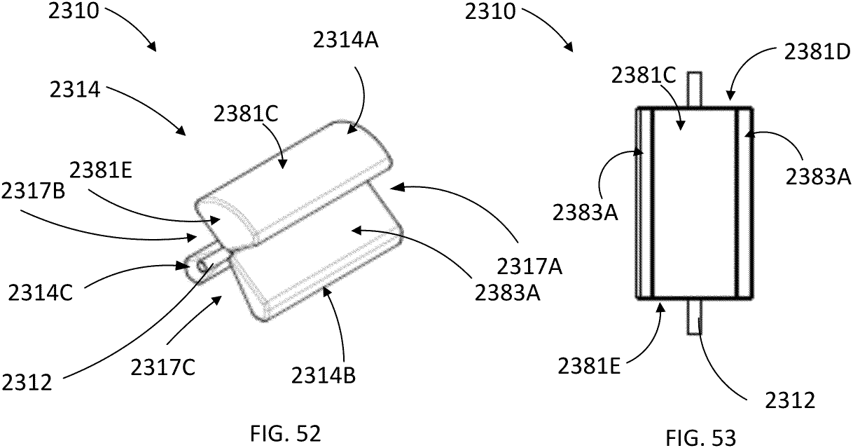

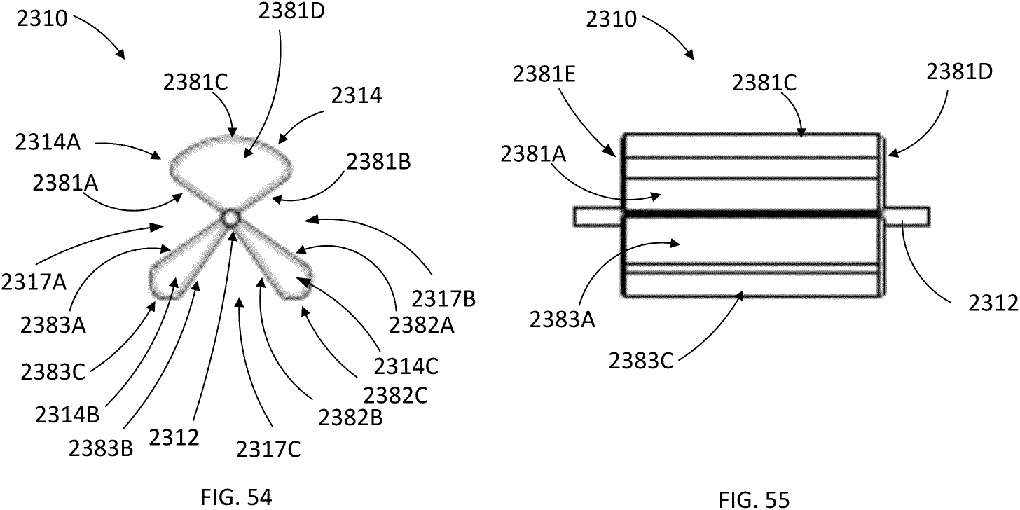

FIGS. 52-55 are a perspective view, a top view, a distal end view, and a side view, respectively, of an inflation assembly 2310 in an expanded configuration. The inflation assembly 2310 can be similar in structure and/or function to any of the inflation assembly systems and devices described herein, such as the inflation assembly 110 of system 100 and/or the inflation assembly 1510 of system 1500. For example, the inflation assembly 2310 can include an elongated tube 2312 and an inflatable member 2314. The elongated tube 2312 can define an inflation lumen in fluid communication with an interior of the inflatable member 2314 such that fluid can be delivered to and/or drawn from the interior of the inflatable member 2314 to transition the inflatable member 2314 between an unexpanded or undeployed (e.g., uninflated) configuration and the expanded or deployed (e.g. inflated) configuration. The inflatable member 2314 can be disposed along a portion of the elongated tube 2312 (e.g., near or at a distal end of the elongated tube 2312).

The inflatable member 2314 defines passageways 2317 (e.g., a first passageway 2317A, a second passageway 2317B, and a third passageway 2317C) such that fluid (e.g., air) can travel through the passageways 2317 when the inflatable member 2314 is in the expanded configuration. In the expanded configuration, when the inflatable member 2314 is disposed in an upper trachea of a patient, each of the first passageway 2317A, the second passageway 2317B, and the third passageway 2317C can be partially bounded by the inflatable member 2314 and partially bounded by the patient’s tracheal wall. In some embodiments, the elongated tube 2312 can bound a portion of one or more of the first passageway 2317A, the second passageway 2317B, and the third passageway 2317C. Thus, air can flow between a portion of the trachea on a first side of the inflatable member 2314 (e.g., a region between a cricoid ring of the patient and the inflatable member 2314) and a portion of the trachea on a second side of the inflatable member 2314 (e.g., a region between the inflatable member 2314 and the lungs of the patient) through the passageways 2317 such that the patient can ventilate through the passageways 2317 and oxygenate when the inflatable member 2314 is in the expanded configuration.

The inflatable member 2314 can include a top portion 2314A (also referred to as a first portion), a first bottom portion 2314B (also referred to as a second portion), and a second bottom portion 2314C (also referred to as a third portion). In the expanded configuration, each of the top portion 2314A, the first bottom portion 2314B, and the second bottom portion 2314B can be formed as a wedge. For example, the top portion 2314A can have a first side 2381A, a second side 2381B, and an arcuate side 2381C. The first bottom portion 2314B can have a first side 2383A, a second side 2383B, and an arcuate side 2383C. The second bottom portion 2314C can have a first side 2382A, a second side 2382B, and an arcuate side 2382C. The inflatable member 2314 can also have a distal end 2381D and a proximal end 2381E.

The first passageway 2317A can be partially defined by the first side 2381A of the top portion 2314A and a first side 2383A of the first bottom portion 2314B. The second passageway 2117B can be partially defined by the second side 2381B of the top portion 2314A and the first side 2382B of the second bottom portion 2114B. The third passageway 2117C can be partially defined by the second side 2382B of the first bottom portion 2314B and the second side 2382B of the second bottom portion 2314C. In some embodiments, when in the expanded configuration, the inflatable member 2314 can be shaped and sized such that the first bottom portion 2314B and the second bottom portion 2314C (e.g., the arcuate side 2383C and the arcuate side 2382C) can contact and be constrained by the posterior tracheal wall, urging the top portion 2314A against the anterior tracheal wall to apply coaptive pressure against the anterior tracheal wall.

In some embodiments, the top portion 2314A, the first bottom portion 2314B, and the second bottom portion 2314C are in fluidic communication with each other such that the inflation lumen of the elongated tube 2312 can be used to provide fluid to all of the top portion 2314A, the first bottom portion 2314B, and the second bottom portion 2314C. In some embodiments, the top portion 2314A, the first bottom portion 2314B, and the second bottom portion 2314C are fluidically isolated from each other such that separate inflation lumens and/or separate ports of the elongated tube 2312 are used to provide fluid to the top portion 2314A, the first bottom portion 2314B, and the second bottom portion 2314C.

In some embodiments, the inflatable member 2314 can be the same or similar in structure and/or function to any of the inflatable members described herein, such as the inflatable member 1514. For example, the inflatable member 2314 can be formed of any suitable material having characteristics described with respect to other inflatable members described herein. The inflation assembly 2310 may also include a magnetic member (not shown) that can be the same or similar in structure and/or function to any of the magnetic members described herein. For example, in some embodiments, the elongated member 2312 can define a central lumen within which the magnetic member can be disposed. In some embodiments, the magnetic member can be centered between a first end and a second end of the inflatable member 2314. In some embodiments, the magnetic member can be the same or similar in structure and/or function to any of the magnetic members described herein, such as the magnetic member 1815. For example, the magnetic member can be configured to be urged toward an external magnetic assembly (such as the external magnetic assembly 1540) when disposed in the patient’s upper trachea. Furthermore, in some embodiments, the magnetic member can be coupled to any suitable portion of the inflation assembly 2310 (e.g., an outer surface of an inner tubular member disposed within the elongated tube 2312, directly to the inflatable member 2314 such as at the end(s) and/or along an inner or outer surface of the inflatable member 2314, and/or to the elongated tube 2312). In some embodiments, the magnetic member can be optionally not included in the inflation assembly 2310 and coaptation between the inflatable member 2314 and the anterior neck portion of the patient can be achieved under only the pressure of the fluid inside the inflatable member 2314. Additionally, in some embodiments the inflation lumen can be defined between an inner tubular member (e.g., inner tubular member 1684) and the elongated tube 2312 such that the inflation lumen surrounds the inner tubular member. In use, the inflation assembly 2310 can be operated the same or similarly as described with respect to other inflation assemblies described herein, such as the inflation assembly 1510 and the inflation assembly 1810. For example, when a portion of the inflatable member 2314 (e.g., the arcuate side 2381C of the top portion 2314A) is in coaptive contact with the anterior tracheal wall of the patient, the inflatable member 2314 can be visualized (e.g. via ultrasound) and a needle can pierce the inflatable member 2314 such that a procedure the same or similar as described with respect to FIGS. 3A-3M and/or FIGS. 18-22 can be performed.

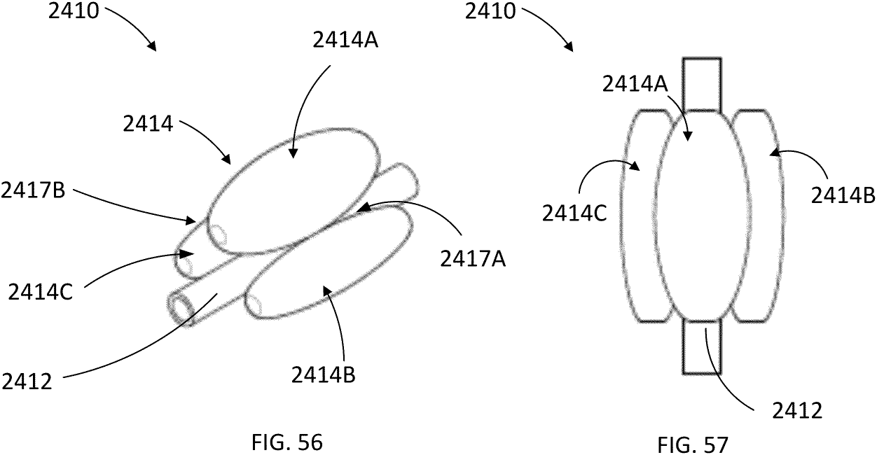

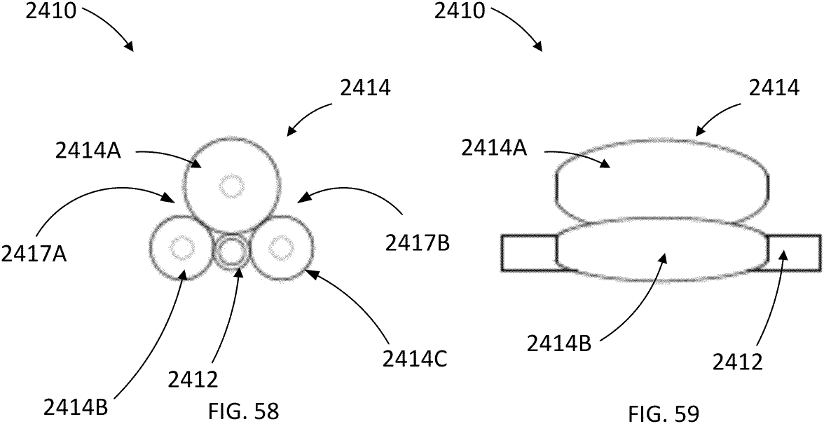

FIGS. 56-59 are a perspective view, a top view, a distal end view, and a side view, respectively, of an inflation assembly 2410 in an expanded configuration. The inflation assembly 2410 can be similar in structure and/or function to any of the inflation assembly systems and devices described herein, such as the inflation assembly 110 of system 100 and/or the inflation assembly 1510 of system 1500. For example, the inflation assembly 2410 can include an elongated tube 2412 and inflatable members 2414. The elongated tube 2412 can define an inflation lumen in fluid communication with an interior of the inflatable members 2414 such that fluid can be delivered to and/or drawn from the interior of the inflatable members 2414 to transition the inflatable members 2414 between an unexpanded or undeployed (e.g., uninflated) configuration and the expanded or deployed (e.g. inflated) configuration. The inflatable members 2414 can be disposed along a portion of the elongated tube 2412 (e.g., near or at a distal end of the elongated tube 2412).

The inflatable members 2414 include a first inflatable member 2414A, a second inflatable member 2414B, and a third inflatable member 2414C. The first inflatable member 2414A and the second inflatable member 2414B define a first passageway 2417A and the first inflatable member 2414A and the third inflatable member 2414C define a second passageway 2417B such that fluid (e.g., air) can travel through the passageways 2417A, 2417B when the inflatable members 2414 are in the expanded configuration. In the expanded configuration, when the inflatable members 2414 are disposed in an upper trachea of a patient, each of the first passageway 2417A and the second passageway 2417B can be partially bounded by the inflatable members 2414 and partially bounded by the patient’s tracheal wall. In some embodiments, the elongated tube 2412 can bound a portion of one or more of the first passageway 2417A and the second passageway 2417B. Thus, air can flow between a portion of the trachea on a first side of the inflatable members 2414 (e.g., a region between a cricoid ring of the patient and the inflatable members 2414) and a portion of the trachea on a second side of the inflatable members 2414 (e.g., a region between the inflatable members 2414 and the lungs of the patient) through the passageways 2417A, 2417B such that the patient can ventilate through the passageways 2417A, 2417B and oxygenate when the inflatable members 2414 are in the expanded configuration.

The second inflatable member 2414B and the third inflatable member 2414C can be disposed below the first inflatable member 2414A. In some embodiments, a central axis of the second inflatable member 2414B and a central axis of the third inflatable member 2414C can be parallel to each other. In some embodiments, a central axis of the second inflatable member 2414B and a central axis of the third inflatable member 2414C can be parallel to each other and disposed in a plane containing a central axis of the elongated tube 2412 (e.g., equidistant from the central axis of the elongated tube 2412). In some embodiments, the first inflatable member 2414A can be formed of a different material than the second inflatable member 2414B and the third inflatable member 2414C. For example, in some embodiments, the first inflatable member 2414A can be formed of a material that has a different compliance and/or flexibility compared to the second inflatable member 2414B and/or the third inflatable member 2414C. For example, the first inflatable member 2414A can be formed of a material that is more compliant and/or more flexible compared to the second inflatable member 2414B and/or the third inflatable member 2414C. Thus, as the first inflatable member 2414A, the second inflatable member 2414B, and the third inflatable member 2414C are inflated, the first inflatable member 2414A can continue to expand after the second inflatable member 2414B and the third inflatable member 2414C are fully inflated such that the second inflatable member 2414B and the third inflatable member 2414C are constrained by the posterior tracheal wall and the first inflatable member 2414A expands and is urged against the anterior tracheal wall to apply coaptive pressure against the anterior tracheal wall.

In some embodiments, the first inflatable member 2414A, the second inflatable member 2414B, and the third inflatable member 2414C are in fluidic communication with each other such that the inflation lumen of the elongated tube 2412 can be used to provide fluid to all of the first inflatable member 2414A, the second inflatable member 2414B, and the third inflatable member 2414C. In some embodiments, the first inflatable member 2414A, the second inflatable member 2414B, and the third inflatable member 2414C are fluidically isolated from each other such that separate inflation lumens and/or separate ports of the elongated tube 2412 are used to provide fluid to the first inflatable member 2414A, the second inflatable member 2414B, and the third inflatable member 2414C.

In some embodiments, the inflatable members 2414 (e.g., the first inflatable member 2414A) can be the same or similar in structure and/or function to any of the inflatable members described herein, such as the inflatable member 1514. For example, the inflatable members 2414 can be formed of any suitable material having characteristics described with respect to other inflatable members described herein. The inflation assembly 2410 may also include a magnetic member (not shown) that can be the same or similar in structure and/or function to any of the magnetic members described herein. For example, in some embodiments, the elongated member 2412 can define a central lumen within which the magnetic member can be disposed. In some embodiments, the magnetic member can be centered between a first end and a second end of one or more of the inflatable members 2414. In some embodiments, the magnetic member can be the same or similar in structure and/or function to any of the magnetic members described herein, such as the magnetic member 1815. For example, the magnetic member can be configured to be urged toward an external magnetic assembly (such as the external magnetic assembly 1540) when disposed in the patient’s upper trachea. Furthermore, in some embodiments, the magnetic member can be coupled to any suitable portion of the inflation assembly 2410 (e.g., an outer surface of an inner tubular member disposed within the elongated tube 2412, directly to the inflatable members 2414 such as at the end(s) and/or along an inner or outer surface of one or more of the inflatable members 2414, and/or to the elongated tube 2412). Additionally, in some embodiments the inflation lumen can be defined between an inner tubular member (e.g., inner tubular member 1684) and the elongated tube 2412 such that the inflation lumen surrounds the inner tubular member. In some embodiments, the magnetic member can be optionally not included in the inflation assembly 2410 and coaptation between the first inflatable member 2314A and the anterior neck portion of the patient can be achieved under only the pressure of the fluid inside the inflatable members 2414. In use, the inflation assembly 2410 can be operated the same or similarly as described with respect to other inflation assemblies described herein, such as the inflation assembly 1510 and the inflation assembly 1810. For example, when a portion of the first inflatable member 2414A is in coaptive contact with the anterior tracheal wall of the patient, the first inflatable member 2414A can be visualized (e.g. via ultrasound) and a needle can pierce the first inflatable member 2414A such that a procedure the same or similar as described with respect to FIGS. 3A-3M and/or FIGS. 18-22 can be performed.

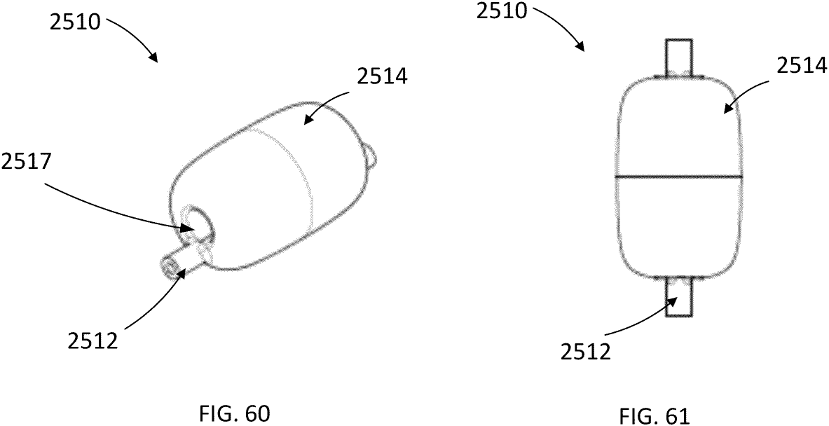

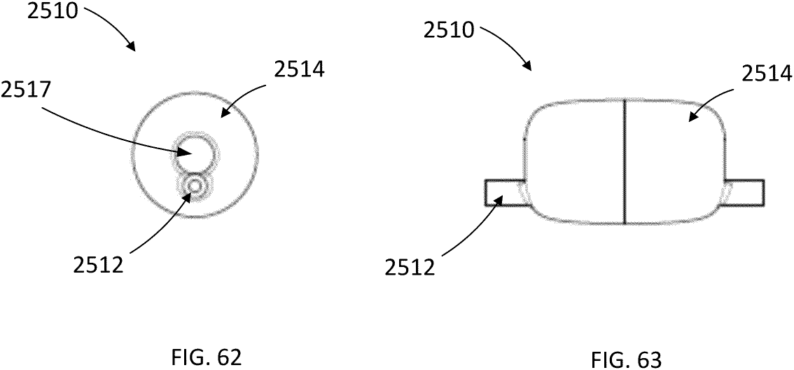

FIGS. 60-63 are a perspective view, a top view, a distal end view, and a side view, respectively, of an inflation assembly 2510 in an expanded configuration. The inflation assembly 2510 can be similar in structure and/or function to any of the inflation assemblies described herein, such as the inflation assembly 110 of the system 100 and/or the inflation assembly 1510 of the system 1500. For example, the inflation assembly 2510 can include an elongated tube 2512 and an inflatable member 2514. The elongated tube 2512 defines an inflation lumen in fluid communication with an interior of the inflatable member 2514 such that fluid can be delivered to and/or drawn from the interior of the inflatable member 2514 to transition the inflatable member 2514 between an unexpanded or undeployed (e.g., uninflated) configuration and an expanded or deployed (e.g., inflated) configuration. As shown, the inflatable member 2514 can be disposed on or near a distal end of the elongated tube 2512.

As shown, the inflatable member 2514 can have a toroidal shape and can define a passageway 2517 (also referred to as a “vent”) extending through the inflatable member 2514 such that fluid (e.g., air) can pass through the inflatable member 2514 when the inflatable member 2514 is disposed within a trachea of a patient in the expanded configuration. The diameter of the passageway 2517 can be sufficiently large such that a patient can be properly ventilated (e.g., freely or via a ventilator) through the passageway 2517 when the inflatable member 2514 is disposed in a trachea of the patient and the outer surface of the inflatable member 2514 contacts the inner wall of the trachea of the patient (e.g., in an uninterrupted manner such that air cannot flow along an outer surface of the inflatable member 2514 or the rate that air can flow along an outer surface of the inflatable member 2514 is insufficient for proper ventilation and oxygenation). In some embodiments, a central axis of the passageway 2517 can be coaxial with a central axis of the inflatable member 2514. In some embodiments, the elongated tube 2512 can be disposed on a first side of the passageway 2517 and can have a central axis parallel to the central axis of the passageway 2517.

Although FIGS. 60-63 show the inflatable member 2514 as defining one passageway 2517, the inflatable member 2514 can define any suitable number of passageways 2517 (e.g., two, three, four, or more passageways 2517). Although the passageway 2517 are shown as being cylindrical, the passageway 2517 can have any suitable shape. In some embodiments, the inflatable member 2514 can define a suitable number of passageways 2517 having a suitable size (e.g., having a suitable combined cross-sectional area) such that a patient can be properly ventilated (e.g., freely or via a ventilator) through the passageways 2517 when the inflatable member 2514 is disposed in a trachea of the patient and the outer surface of the inflatable member 2514 contacts the inner wall of the trachea of the patient (e.g., in an uninterrupted manner such that air cannot flow along an outer surface of the inflatable member 2514 or the rate that air can flow along an outer surface of the inflatable member 2514 is insufficient for proper ventilation and oxygenation).

In some embodiments, the inflatable member 2514 can be the same or similar in structure and/or function to any of the inflatable members described herein, such as the inflatable member 1614. For example, the inflatable member 2514 can be formed of any suitable material having characteristics described with respect to other inflatable members described herein. In some embodiments, the inflation assembly 2510 does not include a magnetic member, as the inflatable member 2514 can inflate to a sufficiently large size such that a bottom portion of the inflatable member 2514 (e.g., a portion of the outer surface of the inflatable member opposite the passageway 2517 relative to the elongated tube 2512) can contact and be constrained by the posterior tracheal wall, urging a top portion of the inflatable member 2514 (e.g., a portion of the outer surface opposite the elongated tube 2512 relative to the passageway 2517) to apply coaptive pressure against an anterior tracheal wall.

In some embodiments, the inflation assembly 2410 may optionally include a magnetic member (not shown) that can be the same or similar in structure and/or function to any of the magnetic members described herein. For example, in some embodiments, the elongated member 2512 can define a central lumen within which the magnetic member can be disposed. In some embodiments, the magnetic member can be centered between a first end and a second end of the inflatable member 2514. In some embodiments, a magnetic member can be included that can be the same or similar in structure and/or function to any of the magnetic members described herein, such as the magnetic member 1515. For example, the magnetic member can be configured to be urged toward an external magnetic assembly (such as the external magnetic assembly 1540) when disposed in the patient’s upper trachea. In some embodiments, the magnetic member can be coupled to any suitable portion of the inflation assembly 2510 (e.g., an outer surface of an inner tubular member disposed within the elongated tube 2512, directly to the inflatable member 2514 such as at the end(s) and/or along an inner or outer surface of the inflatable member 2514, and/or to the elongated tube 2512). Additionally, in some embodiments the inflation lumen can be defined between an inner tubular member (e.g., inner tubular member 1684) and the elongated tube 2512 such that the inflation lumen surrounds the inner tubular member. In use, the inflation assembly 2510 can be operated the same or similarly as described with respect to other inflation assemblies described herein, such as the inflation assembly 1510. For example, when a portion of the inflatable member 2514 is in coaptive contact with the anterior tracheal wall of the patient, the inflatable member 2514 can be visualized (e.g. via ultrasound) and a needle can pierce the inflatable member 2514 such that a procedure the same or similar as described with respect to FIGS. 3A-3M and/or FIGS. 18-22 can be performed.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

Where schematics and/or embodiments described above indicate certain components arranged in certain orientations or positions, the arrangement of components may be modified. While the embodiments have been particularly shown and described, it will be understood that various changes in form and details may be made. Any portion of the apparatus and/or methods described herein may be combined in any combination, except mutually exclusive combinations. The embodiments described herein can include various combinations and/or sub-combinations of the functions, components, and/or features of the different embodiments described.

1. A system, comprising:
an elongated tube;
an inflatable member coupled to the end of the elongated tube, the inflatable member configured to transition from an uninflated configuration to an inflated configuration such that the inflatable member presses against the anterior wall of the trachea in the inflated configuration to enhance ultrasound visualization of tissue between the patient’s skin and the anterior wall of the trachea, the inflatable member defining a passageway extending from a first end of the inflatable member to a second end of the inflatable member such that air can travel through the trachea via the passageway when the inflatable member is in the inflated configuration and pressing against the anterior wall of the trachea.

2. The system of claim 1, wherein the passageway is entirely defined by the inflatable member.

3. The system of claim 1, wherein the passageway is defined partially be the inflatable member and partially be a portion of a wall of the trachea.

4. The system of claim 1, wherein the passageway is a first passageway, the inflatable member defining a second passageway extending from the first end of the inflatable member to the second end of the inflatable member such that air can travel through the trachea via the second passageway when the inflatable member is in the inflated configuration and pressing against the anterior wall of the trachea.

5. The system of claim 1, further comprising:
a guidewire assembly including a guidewire and a coupling member, the guidewire assembly configured to be translated through an opening in an anterior neck of a patient such that the coupling member engages with the inflatable member and the guidewire extends through the anterior neck of the patient.

6. The system of claim 1, wherein the inflatable member is configured to press against the anterior wall of the trachea in the inflated configuration to enhance ultrasound visualization of tissue between the patient’s skin and the anterior wall of the trachea when the inflatable member is disposed in a space between the anterior tracheal wall and an outside of an endotracheal tube.

7. The system of claim 1, wherein the inflatable member is configured to press against the anterior wall of the trachea in the inflated configuration to enhance ultrasound visualization of tissue between the patient’s skin and the anterior wall of the trachea when the inflatable member is disposed spaced beyond an end of an endotracheal tube.

8. The system of claim 1, further comprising:
a magnetic member configured such that, in response to disposing an external magnetic assembly on a surface of the anterior neck of the patient, the inflatable member can be urged against an inner surface of the tracheal wall of an upper trachea such that any intervening structure between the surface of the anterior neck of the patient and the inner surface of the tracheal wall is disposed between the external magnetic assembly and the inflatable member with substantially no fluid gaps.

9. The system of claim 8, wherein the magnetic member is disposed within the inflatable member.

10. A method, comprising:
translating an inflatable member coupled to a first end of an elongated tube through an orifice of a patient, through a cricoid ring of the patient, and into a trachea of the patient; and
inflating the inflatable member via a lumen of the elongated tube such that the inflatable member transitions from an uninflated configuration to an inflated configuration, the inflatable member in the inflated configuration pressing against an anterior wall of the trachea to enhance ultrasound visualization of tissue between the patient’s skin and the anterior wall of the trachea and defining a passageway extending from a first end of the inflatable member to a second end of the inflatable member such that air can travel through the trachea via the passageway.

11. The method of claim 10, further comprising:
translating a coupling member of a guidewire assembly through an anterior neck of the patient and into the upper trachea of the patient, the guidewire assembly including a guidewire having a first end coupled to the coupling member and a second end disposed outside the patient, the guidewire extending through the anterior neck of the patient.

12. The method of claim 10, wherein the translating includes translating the first end of the elongated tube into the trachea of the patient such that the inflatable member is disposed in a space between the anterior wall of the trachea and an outside of an endotracheal tube.

13. The method of claim 10, wherein the passageway is entirely defined by the inflatable member.

14. The method of claim 10, wherein the passageway is defined partially be the inflatable member and partially be a portion of a wall of the trachea.

15. The method of claim 10, wherein the passageway is a first passageway, the inflatable member defining a second passageway extending from the first end of the inflatable member to the second end of the inflatable member such that air can travel through the trachea via the second passageway when the inflatable member is in the inflated configuration and pressing against the anterior wall of the trachea.

16. A method, comprising:
translating a first end of an elongated tube through an orifice of a patient, through a cricoid ring of the patient, and into a trachea of the patient such that an inflatable member coupled to the elongated tube is disposed in a space between a tracheal wall and an outside of an endotracheal tube;
inflating the inflatable member via a lumen of the elongated tube such that the inflatable member transitions from an uninflated configuration to an inflated configuration, the inflatable member in the inflated configuration pressing against the anterior wall of the trachea and enhancing ultrasound visualization of tissue between the patient’s skin and the anterior wall of the trachea; and
translating a coupling member of a guidewire assembly through an anterior neck of the patient and into the upper trachea of the patient, the guidewire assembly including a guidewire having a first end coupled to the coupling member and a second end disposed outside the patient, the guidewire extending through the anterior neck of the patient.

17. The method of claim 16, further comprising coupling the coupling member to the inflatable member.

18. The method of claim 16, further comprising disposing an external magnetic assembly on a surface of the anterior neck of the patient such that a magnetic member of the elongated tube is urged toward the anterior neck of the patient and the inflatable member is disposed between the endotracheal tube and the anterior neck of the patient.

19. The method of claim 16, further comprising disposing an external magnetic assembly on a surface of the anterior neck of the patient such that a magnetic member of the elongated tube is urged toward the anterior neck of the patient and the inflatable member is disposed against an inner surface of the trachea wall of the upper trachea such that any intervening structure between the surface of the anterior neck of the patient and the inner surface of the trachea wall is disposed between the external magnetic assembly and the inflatable member with substantially no fluid gaps.

20. The method of claim 16, further comprising inserting a first end of a needle through the anterior neck, into the trachea, and through a sidewall of the inflatable member, the translating of the coupling member of the guidewire assembly being at least partially through a lumen of the needle.

21. The method of claim 16, wherein inflating the inflatable member causes the inflatable member to press against the outside of the endotracheal tube.

22. The method of claim 16, further comprising translating the coupling member of the guidewire assembly relative to the endotracheal tube through the cricoid ring and out of the orifice of the patient.

23. The method of claim 22, further comprising:
translating the endotracheal tube through the cricoid ring and out of the orifice of the patient; and
disposing a tracheostomy tube within an opening of the anterior neck of the patient through which the guidewire was disposed.

24. The method of claim 16, wherein the elongated tube and the inflatable member are a Foley catheter.

25. A system, comprising:
an elongated tube;
an inflatable member coupled to the end of the elongated tube, the inflatable member configured to transition from an uninflated configuration to an inflated configuration such that, when the inflatable member is disposed in a space between a tracheal wall and an outside of an endotracheal tube, the inflatable member presses against the anterior wall of the trachea in the inflated configuration such that ultrasound visualization of tissue between the patient’s skin and the anterior wall of the trachea can be enhanced;
a guidewire assembly including a guidewire and a coupling member, the guidewire assembly configured to be translated through an opening in an anterior neck of a patient such that the coupling member engages with the inflatable member and the guidewire extends through the anterior neck of the patient.

26. The system of claim 25, further comprising:
a puncture dilator configured to be translated over the guidewire to dilate the opening within which the guidewire is disposed.

27. The system of claim 26, wherein the guidewire is a first guidewire, further comprising a second guidewire, the puncture dilator defining a first lumen and a second lumen, the first lumen configured to receive the first guidewire, the second guidewire configured to be threaded along the second guidewire.

28. The system of claim 27, further comprising:
a tracheal tract dilator configured to be translated along the second guidewire disposed within the opening in the anterior neck after removal of the puncture dilator.

29. The system of claim 25, wherein the inflatable member includes an inner balloon and an outer balloon, the inner balloon disposed within the outer balloon, the elongated tube defining a first inflation lumen and a second inflation lumen, the inner balloon fluidically coupled to the first inflation lumen and the outer balloon fluidically coupled to the second inflation lumen of the elongated tube.

30. The system of claim 29, wherein the inflatable member includes a set of struts configured to maintain a space between the inner balloon and the outer balloon.

31. The system of claim 25, wherein the inflatable member is sufficiently large in the inflated configuration such that the inflatable member presses against the tracheal wall and the endotracheal tube when disposed in the space between the tracheal wall and the outside of the endotracheal tube in the inflated configuration.

32. The system of claim 25, further comprising:
a magnetic member coupled to the elongated tube, the magnetic member configured such that, in response to disposing an external magnetic assembly on a surface of the anterior neck of the patient, the inflatable member can be urged against an inner surface of the trachea wall of an upper trachea such that any intervening structure between the surface of the anterior neck of the patient and the inner surface of the trachea wall is disposed between the external magnetic assembly and the inflatable member with substantially no fluid gaps.

33. The system of claim 25, further comprising:
a rigid stylet disposable within the elongated tube during insertion of the elongated tube through a patient’s vocal cords and into an upper trachea of the patient, the rigid stylet including a curved portion.
